(19) United States
(12) Patent Application Publication
(10) Pub. No.: US 2026/0306680 A1
(43) Pub. Date: Oct. 8, 2026

(54) RADIOLABELED COMPOUNDS TARGETING THE PROSTATE-SPECIFIC MEMBRANE ANTIGEN

(71) Applicants: PROVINCIAL HEALTH SERVICES AUTHORITY, Vancouver (CA); THE UNIVERSITY OF BRITISH COLUMBIA, Vancouver (CA)

(72) Inventors: Kuo-Shyan LIN, Richmond (CA); François BÉNARD, Vancouver (CA); Zhengxing ZHANG, Vancouver (CA); Fei Yuan LU, Vancouver (CA)

(21) Appl. No.: 18/996,309

(22) PCT Filed: Jul. 17, 2023
(86) PCT No.: PCT/CA2023/050959

§ 371 Date: Jan. 17, 2025

Related U.S. Application Data
(60) Provisional application No. 63/390,207, filed on Jul. 18, 2022.

(51) Int. Cl. A61K 51/08

(52) U.S. Cl. CPC A61K 51/088

(57) ABSTRACT

The present disclosure relates to prostate specific membrane antigen (PSMA)-targeting compounds of formula (I) or of a salt or a solvate thereof. The PSMA-targeting compounds of the present disclosure can be useful as imaging agents for PSMA-expressing diseases/conditions.

### CROSS-REFERENCE TO RELATED APPLICATIONS

This application claims priority to U.S. Provisional Application No. 63/390,207, filed Jul. 18, 2022, the disclosure of which is herein incorporated by reference in its entirety for all purposes.

### FIELD OF INVENTION

The present invention relates to radiolabeled compounds for in vivo imaging or treatment of diseases or conditions characterized by expression of prostate-specific membrane antigen, particularly compounds with low uptake in salivary glands and/or kidneys.

### BACKGROUND OF THE INVENTION

Prostate cancer is the second most prevalent cancer diagnosis in men with a GLOBOCAN estimate of 1,276,106 new cases reported in 2018 and is the fifth leading cause of death worldwide. (Rawla P., Epidemiology of Prostate Cancer. World J Oncol. 2019 April; 10(2):63-89). Its prevalence continues to trend upward as its mortality and morbidity decline due to widespread use of prostate specific antigen (PSA) testing allowing for early intervention and treatment of the disease. While common usage of PSA testing aids tremendously in diagnosis and staging, its prognostic and predictive properties are limited due to its lack of specificity. (Murphy G et al., Anticancer Res. 1995 July-August; 15(4):1473-9; Murphy G. P. et al., Cancer. 1996 Aug. 15; 78(4):809-18). Furthermore, another highly expressed prostate cancer biomarker, prostate-specific membrane antigen (PSMA), was more capable of predicting and evaluating clinical progression and resistance and hence serves as a better prognostic tool to monitor disease progression and devise corresponding treatment (Murphy G. et al., Anticancer Res. 1995 July-August; 15(4):1473-9; Murphy G. P. et al., Cancer. 1996 Aug. 15; 78(4):809-1; Hupe M. C. et al., Front Oncol. 2018 Dec. 20; 8:623; and Bostwick D. G. et al., Cancer. 1998; 82(11):2256-61). PSMA, also known as folate hydrolase 1 and glutamate carboxypeptidase II, is a transmembrane protein that catalyzes the hydrolysis of N-acetylaspartylglutamate to glutamate and N-acetylaspartate. (Heston W. D. Urology. 1997 March; 49(3):104-112). Its active site is conveniently located on its extracellular domain making it an ideal targeting candidate for both imaging and therapeutic purposes.

Small urea-based molecules such as glutamate-urea-glutamate have been identified to target the catalytic site of glutamate carboxypeptidase II and serve as the basis for many PSMA radiopharmaceuticals including ¹⁸F-PSMA-1007 and the recently FDA-approved ¹⁸F-DCFPyL for imaging of prostate cancer. (Kozikowski A. P., et al., J Med Chem. 2001 Feb. 1; 44(3):298-301.). A limitation of ¹⁸F-DCFPyL is its high liver uptake which reduces its sensitivity for detecting metastatic lesions in liver. ¹⁸F-PSMA-1007 has an even higher nonspecific uptake level in most normal tissues when compared to ¹⁸F-DCFPyL, leading to more false positive diagnosis for this tracer.

Accordingly, there remains a need to develop radiopharmaceuticals with modified pharmacokinetic profiles that enable their excretion through the renal pathway and to reduce background radioactivity for imaging with positron emission tomography (PET).

No admission is necessarily intended, nor should it be construed, that any of the preceding information constitutes prior art against the present invention.

### SUMMARY

A compound of Formula I:

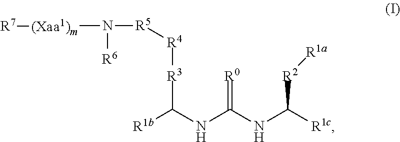

or a salt or a solvate thereof, wherein:
R⁰ is O or S;
R^{1a} is —CO₂H, —SO₂H, —SO₃H, —PO₂H, —PO₃H₂, —OPO₃H₂, —OSO₃H, —B(OH)₂,

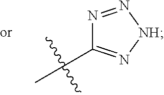 [SMILES: *c1nn[nH]n1]

R^{1b} is —CO₂H, —SO₂H, —SO₃H, —PO₂H, —PO₃H₂, —B(OH)₂, or;

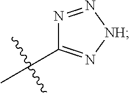 [SMILES: *c1nn[nH]n1]

R^{1c} is —CO₂H, —SO₂H, —SO₃H, —PO₂H, —PO₃H₂, —B(OH)₂, or

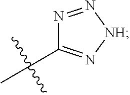 [SMILES: *c1nn[nH]n1]

R² is —CH₂—, —CH(OH)—, —CHF—, —CF₂—, —CH(CH₃)—, —C(CH₃)₂—, —(CH₂)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂—O—CH₂—, —CH₂O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, or —C(O)—NH—C(CH₃)₂—;
R³ is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic C₂-C₂₀ heteroalkylenyl, heteroalkenylenyl, or heteroalkynylenyl;
R⁴ is —O—, —S—, —Se—, —S(O)—, —S(O)₂—, —NHC(O)—, —C(O)NH—,

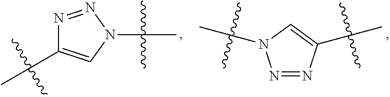 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH—, —NHC(S)O—, —NHC(S)NH—, —NHC(O)C(O)NH—, —S—S—, —S—CH₂—S—, —NH—NH—C(O)—, or —C(O)—NH—NH—;
R⁵ is a linear or branched, cyclic or acyclic, and/or aromatic or non-aromatic C₁-C₃₀ alkylenyl, alkenylenyl or alkynylenyl, or is a linear or branched, cyclic or acyclic, and/or aromatic or non-aromatic C₂-C₃₀ heteroalkylenyl, heteroalkenylenyl or heteroalkynylenyl;
R⁶ is hydrogen, methyl or ethyl;
Xaa¹ is an amino acid of formula —N(R⁸)R⁹C(O)—;
each R⁸ is independently hydrogen or methyl;
each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
wherein at least one R⁹ or R⁵ is —(CH₂)₀₋₃CH(R¹⁰)(CH₂)₀₋₃—;
R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic C₂-C₁₉ heteroalkyl, heteroalkenyl or heteroalkynyl having only 1-3 heteroatoms;
—CH₂R²³, in which R²³ is an optionally substituted C₄-C₁₆ aromatic ring or partially or fully aromatic fused ring system, wherein 0-3 carbons in the aromatic ring or the partially or fully aromatic fused ring system are replaced with N, S and/or O heteroatoms, and wherein the optional substitutions are selected from OH, NH₂, NO₂, halogen, C₁-C₆ alkyl, and/or C₁-C₆ alkoxyl groups; or
selected from:

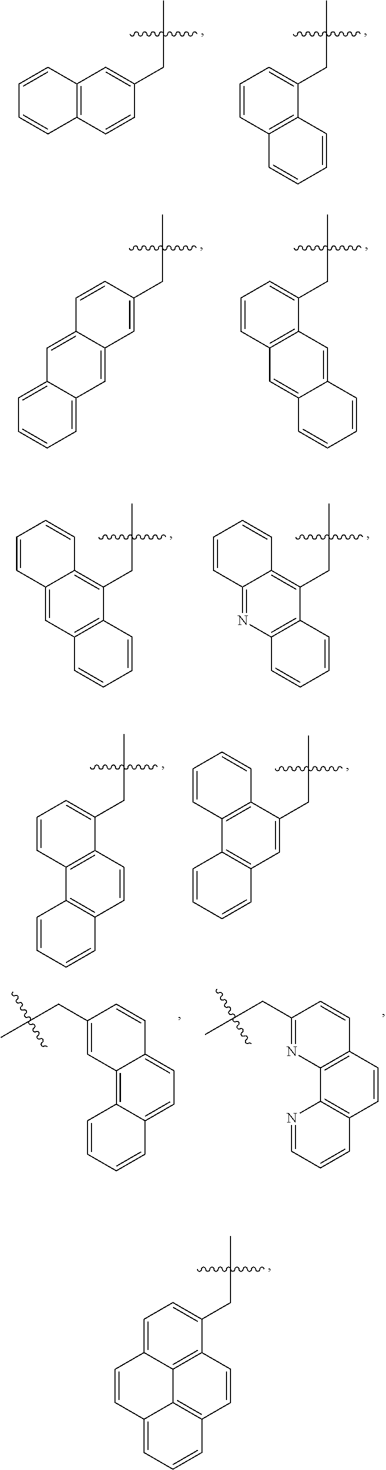

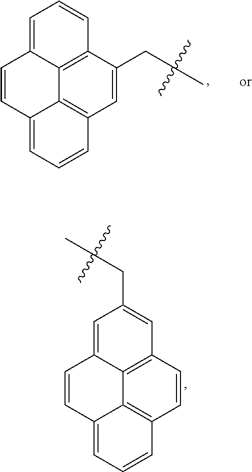 [SMILES: *Cc1cc2cccc3ccc4cccc1c4c32 ; *Cc1cc2ccc3cccc4ccc(c1)c2c34]

optionally modified with one, more than one, or a combination of: halogen, OMe, SMe, NH₂, NO₂, CN, OH, or additional endocyclic ring nitrogen atoms;
R⁷ is

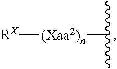

Xaa² is —N(R¹³)R¹⁴C(O)—;
each R¹³ is independently hydrogen or methyl;
each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl; wherein each R¹⁴ is optionally substituted;
wherein each R¹⁴ is optionally substituted with a carboxylic acid, a sulfonic acid, a sulfinic acid, or a phosphoric acid; wherein each R¹⁴ is optionally substituted with one R^{x}; and wherein at least one R¹⁴ is substituted with one R^{X}; and
R^{X} is

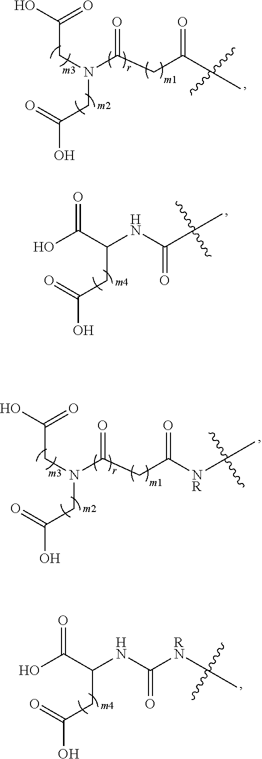

—(C(O))ₚ(CH₂)_{q}-(radiolabeling group), or —(N(R)C(O))ₚ—(CH₂)_{q}-(radiolabeling group), wherein the radiolabeling group independently selected from: an aryl or heteroaryl substituted with a radioisotope or a prosthetic group comprising a trifluoroborate;
R is hydrogen or methyl;
wherein at least one R^{X} is

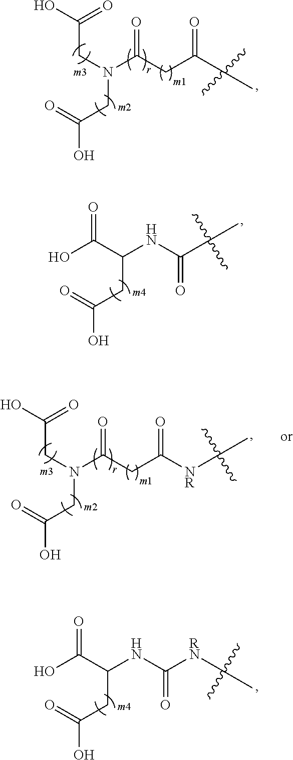

and at least one other R^{X} is —(C(O))ₚ—(CH₂)_{q}-(radiolabeling group) or —(N(R)C(O))ₚ—(CH₂)_{q}-(radiolabeling group);
r is 0 or 1;
n is 1, 2, 3, or 4;
m is 1, 2, 3, or 4;
p is 0 or 1;
q is 0, 1, 2, 3, 4, or 5; and
m1, m2, m3, and m4 are each independently 1, 2, 3, or 4.

In embodiments, the present disclosure relates to methods of imaging prostate specific membrane antigen (PSMA)-expressing tissues in a subject, comprising administering a compound disclosed herein to a subject in need thereof, wherein the compound comprises a positron or gamma emitting radioisotope; and imaging the PSMA-expressing tissue.

### BRIEF DESCRIPTION OF THE DRAWINGS

The features of the invention will become apparent from the following description in which reference is made to the appended drawings wherein:

FIG. 1 shows maximum intensity projection PET images of representative compound ¹⁸F-KL01007-pyridine-BF₃ and ¹⁸F-KL01040 acquired at 1 h post-injection from LNCaP tumor-bearing mice.

FIG. 2 shows maximum intensity projection PET images of ¹⁸F-KL01120 in LNCaP tumor-bearing mice acquired at 1 h post-injection without (left) and with (right) the co-injection of 2-PMPA (0.5 mg).

FIG. 3 shows maximum intensity projection PET images of ¹⁸F-KL01130 in LNCaP tumor-bearing mice acquired at 1 h post-injection without (left) and with (right) the co-injection of 2-PMPA (0.5 mg).

### DETAILED DESCRIPTION

### Definitions

As used herein, the terms “comprising,” “having”, “including” and “containing,” and grammatical variations thereof, are inclusive or open-ended and do not exclude additional, unrecited elements and/or method steps, even if a feature/component defined as a part thereof consists or consists essentially of specified feature(s)/component(s). The term “consisting essentially of” if used herein in connection with a compound, composition, use or method, denotes that additional elements and/or method steps may be present, but that these additions do not materially affect the manner in which the recited compound, composition, method or use functions. The term “consisting of” if used herein in connection with a feature of a composition, use or method, excludes the presence of additional elements and/or method steps in that feature. A compound, composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the context clearly dictates otherwise. The use of the word “a” or “an” when used herein in conjunction with the term “comprising” may mean “one,” but it is also consistent with the meaning of “one or more,” “at least one” and “one or more than one.”

In this disclosure, the recitation of numerical ranges by endpoints includes all numbers subsumed within that range including all whole numbers, all integers and, where suitable, all fractional intermediates (e.g., 1 to 5 may include 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, etcetera.

As used herein, the terms “treat”, “treatment”, “therapeutic” and the like includes ameliorating symptoms, reducing disease progression, improving prognosis and reducing recurrence.

As used herein, the term “diagnostic agent” includes an “imaging agent”. As such, a “diagnostic radiometal” includes radiometals that are suitable for use as imaging agents.

The term “subject” refers to an animal (e.g., a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like). In some embodiments, the subject is a human.

The compounds disclosed herein may also include base-free forms, salts or pharmaceutically acceptable salts thereof. Unless otherwise specified, the compounds claimed and described herein are meant to include all racemic mixtures and all individual enantiomers or combinations thereof, whether or not they are explicitly represented herein.

The compounds disclosed herein may be shown as having one or more charged groups, may be shown with ionizable groups in an uncharged (e.g., protonated) state or may be shown without specifying formal charges. As will be appreciated by the person of skill in the art, the ionization state of certain groups within a compound (e.g., without limitation, CO₂H, PO₃H₂, SO₂H, SO₃H, SO₄H, OPO₃H₂ and the like) is dependent, inter alia, on the pKa of that group and the pH at that location. For example, but without limitation, a carboxylic acid group (i.e., COOH) would be understood to usually be deprotonated (and negatively charged) at neutral pH and at most physiological pH values, unless the protonated state is stabilized. Likewise, OSO₃H (i.e., SO₄H) groups, SO₂H groups, SO₃H groups, OPO₃H₂ (i.e., PO₄H₂) groups and PO₃H groups would generally be deprotonated (and negatively charged) at neutral and physiological pH values.

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g., without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety.

As used herein, a “stereoisomer” refers to a compound made up of the same atoms bonded by the same bonds but having different three-dimensional structures, which are not interchangeable. The present disclosure contemplates various stereoisomers and mixtures thereof and includes enantiomers and diastereomer.

As used herein, the expression “Xy-Xz”, where y and z are integers (e.g. X₁-X₁₅, X₁-X₃₀, X₁-X₁₀₀, and the like), refers to the number of carbons (for alkyls, whether saturated or unsaturated, or aryls) in a compound, R-group or substituent, or refers to the number of carbons plus heteroatoms (for heteroalkyls, whether saturated or unsaturated, or heteroaryls) in a compound, R-group or substituent. Heteroatoms may include any, some or all possible heteroatoms. For example, in some embodiments, the heteroatoms are selected from N, O, S, P and Se. In some embodiments, the heteroatoms are selected from N, O, S and P. Such embodiments are non-limiting. Alkyls and aryls may alternatively be referred to using the expression “Cy-Cz”, where y and z are integers (e.g., C₃-C₁₅ and the like). Further, when the expression “Cy-Cz” is used in association with heteroalkyl, heteroalkenyl, heteroalkynyl, and the like, it is understood that one or more carbon atoms of Cy-Cz alkyl is replaced with a heteroatom, such as N, O, S, P and Se. For example, C4 heteroalkyl can include CH₃CH₂OCH₃.

Unless explicitly stated otherwise, the terms “alkyl” and “heteroalkyl” each includes any reasonable combination of the following: (1) saturated alkyls as well as unsaturated (including partially unsaturated) alkyls (e.g., alkenyls and alkynyls); (2) linear or branched; (3) acyclic or cyclic (aromatic or nonaromatic), the latter of which may include multi-cyclic (fused rings, multiple non-fused rings or a combination thereof); and (4) unsubstituted or substituted. For example, an alkyl or heteroalkyl (i.e., “alkyl/heteroalkyl”) may be saturated, branched and cyclic, or unsaturated, branched and cyclic, or linear and unsaturated, or any other reasonable combination according to the skill of the person of skill in the art. If unspecified, the size of the alkyl/heteroalkyl is what would be considered reasonable to the person of skill in the art. For example, but without limitation, if unspecified, the size of an alkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons in length, subject to the common general knowledge of the person of skill in the art. For example, alkyl can be C₁-C₁₂ alkyl or C₁-C₆ alkyl. Further, but without limitation, if unspecified, the size of a heteroalkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons and heteroatoms in length, subject to the common general knowledge of the person of skill in the art. In the context of the expression “alkyl, alkenyl or alkynyl” and similar expressions, the “alkyl” would be understood to be a saturated alkyl. Likewise, in the context of the expression “heteroalkyl, heteroalkenyl or heteroalkynyl” and similar expressions, the “heteroalkyl” would be understood to be a saturated heteroalkyl.

As used herein, in the context of an alkyl/heteroalkyl group of a compound, the term “linear” may be used as it is normally understood to a person of skill in the art and generally refers to a chemical entity that comprises a skeleton or main chain that does not split off into more than one contiguous chain. Non-limiting examples of linear alkyls include methyl, ethyl, n-propyl, and n-butyl.

As used herein, the term “branched” may be used as it is normally understood to a person of skill in the art and generally refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl group include tert-butyl and isopropyl.

As used herein the term cyclic alkyl/heteroalkyl refers to saturated, unsaturated, or partially unsaturated cycloalkyl and cycloheteroalkyl groups as well as combinations with linear or branched alkyl/heteroalkyl—for example: -(alkylene)₀₋₁-(cycloalkylene)-(alkylene)₀₋₁-, -(alkylene)₀₋₁-(cycloheteroalkylene)-(alkylene)₀₋₁-, -(alkylene)₀₋₁-(arylene)-(alkylene)₀₋₁-, and -(alkylene)₀₋₁-(heteroarylene)-(alkylene)₀₋₁- are included in said term. In an illustrative example, a divalent aromatic heteroalkyl group can be

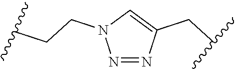 [SMILES: *CCn1cc(C*)nn1]

and a divalent saturated cycloalkyl group can be

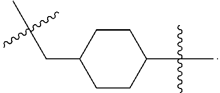 [SMILES: *CC1CCC(*)CC1]

The term “alkylenyl” refers to a divalent analog of an alkyl group. In the context of the expression “alkylenyl, alkenylenyl or alkynylenyl”, “alkylenyl or alkenylenyl” and similar expressions, the “alkylenyl” would be understood to be a saturated alkylenyl. The term “heteroalkylenyl” refers to a divalent analog of a heteroalkyl group. In the context of the expression “heteroalkylenyl, heteroalkenylenyl or heteroalkynylenyl”, “heteroalkylenyl or heteroalkenylenyl” and similar expressions, the “heteroalkylenyl” would be understood to be a saturated heteroalkylenyl.

As used herein, the term “saturated” when referring to a chemical entity may be used as it is normally understood to a person of skill in the art and generally refers to a chemical entity that comprises only single bonds, and may include linear, branched, and/or cyclic groups. Non-limiting examples of a saturated C₁-C₂₀ alkyl group may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, I-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl, t-decyl, cyclopropanyl, cyclobutanyl, cyclopentanyl, cyclohexanyl, cycloheptanyl, cyclooctanyl, cyclononanyl, cyclodecanyl, and the like. The saturated alkyl can be a C₁-C₁₂ saturated alkyl or C₁-C₆ saturated alkyl. Unless otherwise specified, a C₁-C₂₀ alkylenyl therefore encompasses, without limitation, all divalent analogs of the above-listed saturated alkyl groups.

As used herein, the term “unsaturated” when referring to a chemical entity may be used as it is normally understood to a person of skill in the art and generally refers to a chemical entity that comprises at least one double or triple bond, and may include linear, branched, and/or cyclic groups. Non-limiting examples of a C₂-C₂₀ alkenyl group may include vinyl, allyl, isopropenyl, I-propene-2-yl, 1-butene-1-yl, I-butene-2-yl, I-butene-3-yl, 2-butene-1-yl, 2-butene-2-yl, octenyl, decenyl, cyclopropenyl, cyclobutenyl, cyclopentenyl, cyclohexenyl, cycloheptenyl, cyclooctenyl, cyclononanenyl, cyclodecanenyl, and the like. The alkenyl group can be a C₂-C₁₂ alkenyl or C₂-C₆ alkenyl. Unless otherwise specified, a C₁-C₂₀ alkenylenyl therefore encompasses, without limitation, all divalent analogs of the above-listed alkenyl groups. Non-limiting examples of a C₂-C₂₀ alkynyl group may include ethynyl, propynyl, butynyl, pentynyl, hexynyl, heptynyl, octynyl, nonynyl, decynyl, and the like. The alkynyl group can be a C₂-C₁₂ alkynyl or C₂-C₆ alkynyl. Unless otherwise specified, a C₁-C₂₀ alkynylenyl therefore encompasses, without limitation, all divalent analogs of the above-listed alkynyl groups. The alkynylenyl group can be a C₂-C₁₂ alkynyl or C₂-C₆ alkynylenyl. Without limitation, the above-defined saturated C₁-C₂₀ alkyl groups, C₂-C₂₀ alkenyl groups and C₂-C₂₀ alkynyl groups are all encompassed within the term “C₁-C₂₀ alkyl”, unless otherwise indicated. Without limitation, the above-defined saturated C₁-C₂₀ alkylenyl groups, C₂-C₂₀ alkenylenyl groups and C₂-C₂₀ alkynylenyl groups are all encompassed within the term “C₁-C₂₀ alkylenyl”, unless otherwise indicated.

Without limitation, the term “X₁-X₂₀ heteroalkyl” would encompass each of the above-defined saturated C₁-C₂₀ alkyl groups, C₂-C₂₀ alkenyl groups and C₂-C₂₀ alkynyl groups, where one or more of the carbon atoms is independently replaced with a heteroatom. Likewise, without limitation, the term “X₁-X₂₀ heteroalkylenyl” would encompass each of the above-defined saturated C₁-C₂₀ alkylenyl groups, C₂-C₂₀ alkenylenyl groups and C₂-C₂₀ alkynylenyl groups, where one or more of the carbon atoms is independently replaced with a heteroatom. The X₁-X₂₀ heteroalkyl can be a C₁-C₁₂ heteroalkyl or C₁-C₆ heteroalkyl. The person of skill in the art would understand that various combinations of different heteroatoms may be used. Non-limiting examples of non-aromatic heterocyclic groups (can also be referred to as “non-aromatic, cyclic heteroalkyl” in this specification) include aziridinyl, azetidinyl, diazetidinyl, pyrrolidinyl, pyrrolinyl, piperidinyl, piperazinyl, imidazolinyl, pyrazolidinyl, imidazolydinyl, phthalimidyl, succinimidyl, oxiranyl, tetrahydropyranyl, oxetanyl, dioxanyl, thietanyl, thiepinyl, morpholinyl, oxathiolanyl, and the like.

Unless further specified, an “aryl” group includes both single aromatic rings as well as fused rings containing at least one aromatic ring. non-limiting examples of C₃-C₂₀ aryl groups include phenyl (Ph), pentalenyl, indenyl, naphthyl and azulenyl. Non-limiting examples of X₃-X₂₀ aromatic heterocyclic groups (can also be referred to as “heteroaryls” or “aromatic cyclic heteroalkyl” in this specification) include pyrrolyl, imidazolyl, pyrazolyl, pyridinyl, pyridazinyl, pyrimidinyl, pirazinyl, quinolinyl, isoquinolinyl, acridinyl, indolyl, isoindolyl, indolizinyl, purinyl, carbazolyl, indazolyl, phthalazinyl, naphthyridinyl, quinoxalinyl, quinazolinyl, cinnolinyl, pteridinyl, phenanthridinyl, phenazinyl, phenanthrolinyl, perimidinyl, furyl, dibenzofuryl, xanthenyl, benzofuryl, thiophenyl, thianthrenyl, benzothiophenyl, phosphorinyl, phosphinolinyl, phosphindolyl, thiazolyl, oxazolyl, isoxazolyl, and the like.

As used herein, the term “substituted” is used as it would normally be understood to a person of skill in the art and generally refers to a compound or chemical entity that has one chemical group replaced with a different chemical group. Unless otherwise specified, a substituted alkyl is an alkyl in which one or more hydrogen atom(s) are independently each replaced with an atom that is not hydrogen. For example, chloromethyl is a non-limiting example of a substituted alkyl, more particularly an example of a substituted methyl. Aminoethyl is another non-limiting example of a substituted alkyl, more particularly an example of a substituted ethyl. Unless otherwise specified, a substituted compound or group (e.g., alkyl, heteroalkyl, aryl, heteroaryl and the like) may be substituted with any chemical group reasonable to the person of skill in the art. For example, but without limitation, a hydrogen bonded to a carbon or heteroatom (e.g., N) may be substituted with halide (e.g., F, I, Br, Cl), amine, amide, oxo, hydroxyl, thiol, phosphate, phosphonate, sulfate, SO₂H, SO₃H, alkyls, heteroalkyls, aryl, heteroaryl, ketones, carboxaldehyde, carboxylates, carboxamides, nitriles, monohalomethyl, dihalomethyl or trihalomethyl.

As used herein, the term “unsubstituted” is used as it would normally be understood to a person of skill in the art. Non-limiting examples of unsubstituted alkyls include methyl, ethyl, tert-butyl, pentyl and the like. The expression “optionally substituted” is used interchangeably with the expression “unsubstituted or substituted”.

In the structures provided herein, hydrogen may or may not be shown. In some embodiments, hydrogens (whether shown or implicit) may be protium (i.e. ¹H), deuterium (i.e. ²H) or combinations of ¹H and ²H. Methods for exchanging ¹H with ²H are well known in the art. For solvent-exchangeable hydrogens, the exchange of ¹H with ²H occurs readily in the presence of a suitable deuterium source, without any catalyst. The use of acid, base or metal catalysts, coupled with conditions of increased temperature and pressure, can facilitate the exchange of non-exchangeable hydrogen atoms, generally resulting in the exchange of all ¹H to ²H in a molecule.

The term “Xaa” refers to an amino acid residue in a peptide chain or an amino acid that is otherwise part of a compound. Amino acids have both an amino group and a carboxylic acid group, either or both of which can be used for covalent attachment. In attaching to the remainder of the compound, the amino group and/or the carboxylic acid group may be converted to an amide or other structure; e.g. a carboxylic acid group of a first amino acid is converted to an amide (i.e. a peptide bond) when bonded to the amino group of a second amino acid. As such, Xaa may have the formula —N(R^{a})R^{b}C(O)—, where R^{a} and R^{b} are R-groups. R^{a} will typically be hydrogen or methyl. The amino acid residues of a peptide may comprise typical peptide (amide) bonds and may further comprise bonds between side chain functional groups and the side chain or main chain functional group of another amino acid. For example, the side chain carboxylate of one amino acid residue in the peptide (e.g., Asp, Glu, etc.) may be bonded to and the amine of another amino acid residue in the peptide (e.g. Dap, Dab, Orn, Lys). Further details are provided below. Unless otherwise indicated, “Xaa” may be any amino acid, including proteinogenic and nonproteinogenic amino acids. Non-limiting examples of nonproteinogenic amino acids are shown in Table 1 and include: D-amino acids (including without limitation any D-form of the following amino acids), ornithine (Orn), 3-(1-naphtyl)alanine (Nal), 3-(2-naphtyl)alanine (2-Nal), α-aminobutyric acid, norvaline, norleucine (Nle), homonorleucine, beta-(1,2,3-triazol-4-yl)-L-alanine, 1,2,4-triazole-3-alanine, Phe(4-F), Phe(4-Cl), Phe(4-Br), Phe(4-1), Phe(4-NH₂), Phe(4-NO₂), homoarginine (hArg), 2-amino-4-guanidinobutyric acid (Agb), 2-amino-3-guanidinopropionic acid (Agp), β-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-amino-3-(anthracen-2-yl)propanoic acid, 2-amino-3-(anthracen-9-yl)propanoic acid, 2-amino-3-(pyren-1-yl)propanoic acid, Trp(5-Br), Trp(5-OCH₃), Trp(6-F), Trp(5-OH) or Trp(CHO), 2-aminoadipic acid (2-Aad), 3-aminoadipic acid (3-Aad), propargylglycine (Pra), homopropargylglycine (Hpg), beta-homopropargylglycine (Bpg), 2,3-diaminopropionic acid (Dap), 2,4-diaminobutyric acid (Dab), azidolysine (Lys(N₃)), azido-ornithine (Orn(N₃)), 2-amino-4-azidobutanoic acid Dab(N₃), Dap(N₃), 2-(5′-azidopentyl)alanine, 2-(6′-azidohexyl)alanine, 4-amino-1-carboxymethyl-piperidine (Pip), 4-(2-aminoethyl)-1-carboxymethyl-piperazine (Acp), and tranexamic acid. If not specified as an L- or D-amino acid, an amino acid shall be understood to encompass both L- and D-amino acids.

TABLE 1
List of non-limiting examples of non-proteinogenic amino acids.

| | |
|---|---|
| Any D-amino acid of a | 10-aminodecanoic acid |
| proteinogenic amino acid | |
| ornithine (Orn) | 2-aminooctanoic acid |
| 3-(1-naphtyl)alanine (Nal) | 2-amino-3-(anthracen-2- |
| | yl)propanoic acid |
| 3-(2-naphtyl)alanine (2-Nal) | 2-amino-3-(anthracen-9- |
| | yl)propanoic acid |
| α-aminobutyric acid | 2-amino-3-(pyren-1- |
| | yl)propanoic acid |
| norvaline | Trp(5-Br) |
| norleucine (Nle) | Trp(5-OCH₃) |
| homonorleucine | Trp(6-F) |
| beta-(1,2,3-triazol-4-yl)- | Trp(5-OH) |
| L-alanine | |
| 1,2,4-triazole-3-alanine | Trp(CHO) |
| Phe(4-F) or (4-F)Phe | 2-aminoadipic acid (2-Aad) |
| Phe(4-Cl) or (4-Cl)Phe | 3-aminoadipic acid (3-Aad) |
| Phe(4-Br) or (4-Br)Phe | propargylglycine (Pra) |
| Phe(4-1) or (4-1)Phe | homopropargylglycine (Hpg) |
| Phe(4-NH2) or (4-NH₂)Phe | beta-homopropargylglycine (Bpg) |
| Phe(4-NO2) or (4-NO₂)Phe | 2,3-diaminopropionic acid (Dap) |
| (3-I)Tyr | 2,4-diaminobutyric acid (Dab) |
| homoarginine (hArg) | Cysteic acid (CysAcid) |
| homotyrosine (hTyr) | Ne-isopropyl-lysine (Lys(iPr)) |
| 3-(2-phenanthryl)-L-alanine | Arg(Me) |
| (2-(Ant)Ala) | |
| 3-(9-phenanthryl)-L-alanine | Arg(Me)₂ (symmetrical |
| (9-(Ant)Ala) | or asymmetrical) |
| 4-(2-aminoethyl)-1- | azidolysine (Lys(N₃)) |
| carboxymethyl-piperazine (Acp) | |
| 2-(5′-azidopentyl)alanine | azido-ornithine (Orn(N₃)) |
| 2-(6′-azidohexyl)alanine | amino-4-azidobutanoic |
| | acid Dab(N₃) |
| 2-amino-4-guanidinobutyric | tranexamic acid |
| acid (Agb) | |
| 2-amino-3-guanidinopropionic | 4-amino-1-carboxymethyl- |
| acid (Agp) | piperidine (Pip) |
| β-alanine | NH₂(CH₂)₂O(CH₂)₂C(O)OH |
| 4-aminobutyric acid | NH₂(CH₂)₂[O(CH₂)₂]₂C(O)OH |
| 5-aminovaleric acid | NH₂(CH₂)₂[O(CH₂)₂]₃C(O)OH |
| 6-aminohexanoic acid | NH₂(CH₂)₂[O(CH₂)₂]₄C(O)OH |
| 7-aminoheptanoic acid | NH₂(CH₂)₂[O(CH₂)₂]₅C(O)OH |
| 8-aminooctanoic acid | NH₂(CH₂)₂[O(CH₂)₂]₆C(O)OH |
| 9-aminononanoic acid | Ne-acetyl-lysine (Lys(Ac)) |

The wavy line “ [symbol] ” symbol shown through or at the end of a bond in a chemical formula (e.g., in the definitions R⁴, R⁶, R⁷, R⁹ and R¹¹ of Formula I etc.) is intended to define the R group on one side of the wavy line, without modifying the definition of the structure on the opposite side of the wavy line. Where an R group is bonded on two or more sides (e.g., R¹¹), any atoms shown outside the wavy lines are intended to clarify orientation of the R group. As such, only the atoms between the two wavy lines constitute the definition of the R group. Unless specified, chemical groups with more than one point of attachment (such as divalent groups) are understood to be placed in any direction a skilled artisan would understand as chemically possible—e.g., —C(O)NH— and —NHC(O)— are interchangeable unless otherwise noted.

### Compounds

In various aspects, there is disclosed a compound of Formula I (as defined below), Formula II (as defined below), or Formula III (as defined below), including salts solvates, stereoisomers, or mixtures of stereoisomers (each compound being a “compound of the invention”) of the foregoing.

The present disclosure relates to compounds useful as imaging agents and/or therapeutic agents. In some embodiments, the compound of the present disclosure relates to a compound of Formula I:

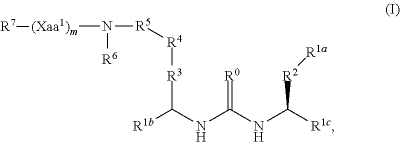

or a salt or a solvate thereof, wherein:
R⁰ is O or S;
R^{1a} is —CO₂H, —SO₂H, —SO₃H, —PO₂H, —PO₃H₂, —OPO₃H₂, —OSO₃H, —B(OH)₂, or

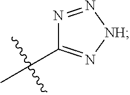 [SMILES: *c1nn[nH]n1]

R^{1b} is —CO₂H, —SO₂H, —SO₃H, —PO₂H, —PO₃H₂, —B(OH)₂, or

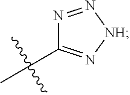 [SMILES: *c1nn[nH]n1]

R^{1c} is —CO₂H, —SO₂H, —SO₃H, —PO₂H, —PO₃H₂, —B(OH)₂, or

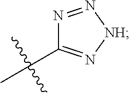 [SMILES: *c1nn[nH]n1]

R² is —CH₂—, —CH(OH)—, —CHF—, —CF₂—, —CH(CH₃)—, —C(CH₃)₂—, —(CH₂)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, or —C(O)—NH—C(CH₃)₂—;
R³ is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic C₂-C₂₀ heteroalkylenyl, heteroalkenylenyl, or heteroalkynylenyl;
R⁴ is —O—, —S—, —Se—, —S(O)—, —S(O)₂—, —NHC(O)—, —C(O)NH—,

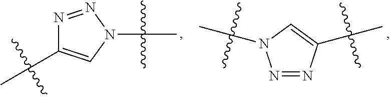 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH—, —NHC(S)O—, —NHC(S)NH—, —NHC(O)C(O)NH—, —S—S—, —S—CH₂—S—, —NH—NH—C(O)—, or —C(O)—NH—NH—;
R⁵ is a linear or branched, cyclic or acyclic, and/or aromatic or non-aromatic C₁-C₃₀ alkylenyl, alkenylenyl or alkynylenyl, or is a linear or branched, cyclic or acyclic, and/or aromatic or non-aromatic C₂-C₃₀ heteroalkylenyl, heteroalkenylenyl or heteroalkynylenyl;
R⁶ is hydrogen, methyl or ethyl;
Xaa¹ is an amino acid of formula —N(R⁸)R⁹C(O)—;
each R⁸ is independently hydrogen or methyl;
each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
wherein at least one R⁹ or R⁵ is —(CH₂)₀₋₃CH(R¹⁰)(CH₂)₀₋₃—;
R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic C₂-C₁₉ heteroalkyl, heteroalkenyl or heteroalkynyl having only 1-3 heteroatoms;
—CH₂R²³, in which R²³ is an optionally substituted C₄-C₁₆ aromatic ring or partially or fully aromatic fused ring system, wherein 0-3 carbons in the aromatic ring or the partially or fully aromatic fused ring system are replaced with N, S and/or O heteroatoms, and wherein the optional substitutions are selected from OH, NH₂, NO₂, halogen, C₁-C₆ alkyl, and/or C₁-C₆ alkoxyl groups; or
selected from:

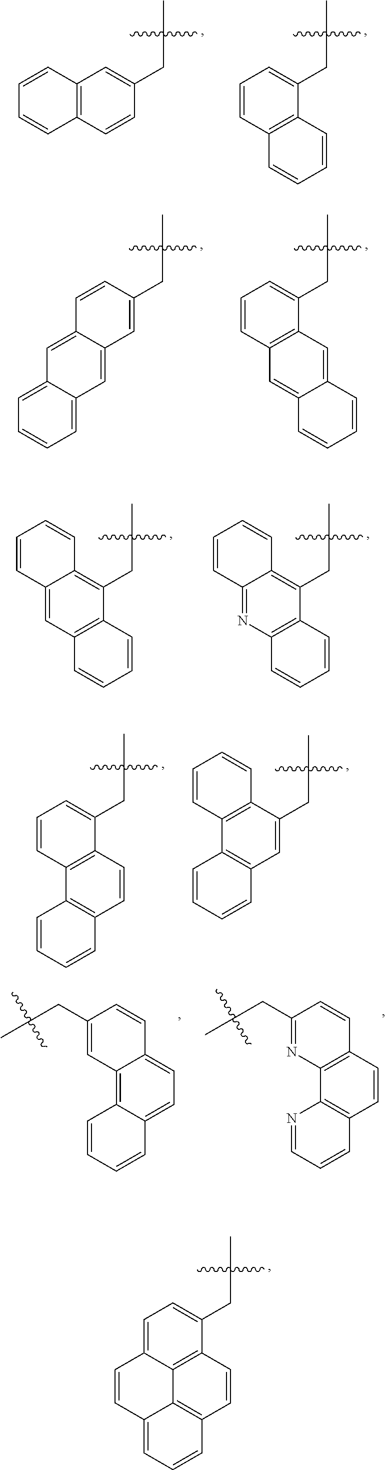

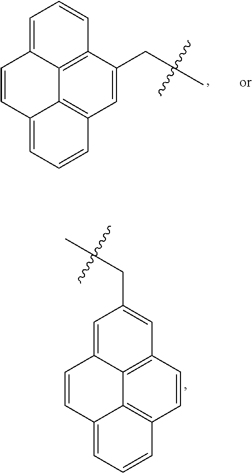 [SMILES: *Cc1cc2cccc3ccc4cccc1c4c32 ; *Cc1cc2ccc3cccc4ccc(c1)c2c34]

optionally modified with one, more than one, or a combination of: halogen, OMe, SMe, NH₂, NO₂, CN, OH, or additional endocyclic ring nitrogen atoms;
R⁷ is

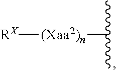

Xaa² is —N(R¹³)R¹⁴C(O)—;
each R¹³ is independently hydrogen or methyl;
each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl, wherein each R¹⁴ is optionally substituted;
wherein each R¹⁴ is optionally substituted with a carboxylic acid, a sulfonic acid, a sulfinic acid, or a phosphoric acid; wherein each R¹⁴ is optionally substituted with one R^{X}; and wherein at least one R¹⁴ is substituted with one R^{X}; and
R^{X} is

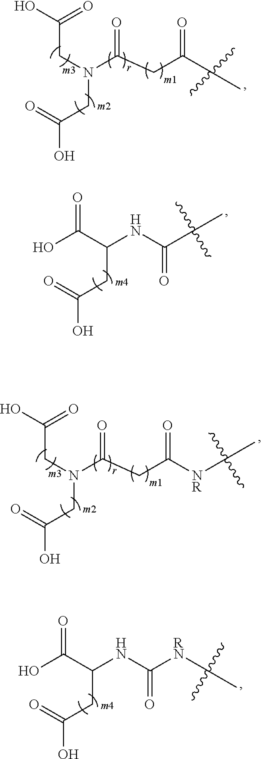

(C(O))ₚ—(CH₂)_{q}-(radiolabeling group), or —(N(R)C(O))ₚ—(CH₂)_{q}-(radiolabeling group), wherein the radiolabeling group independently selected from: an aryl or heteroaryl substituted with a radioisotope or a prosthetic group comprising a trifluoroborate;
R is hydrogen or methyl;
wherein at least one R^{X} is

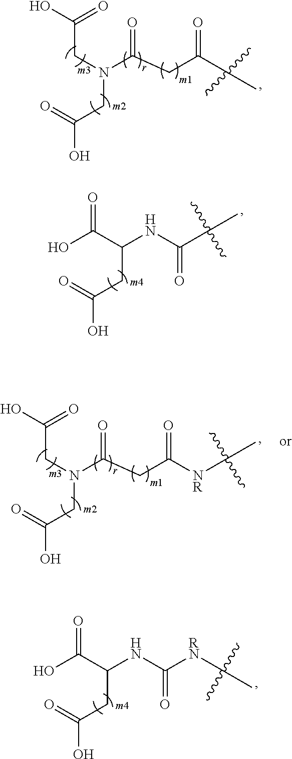

and at least one other R^{X} is —(C(O))ₚ—(CH₂)_{q}-(radiolabeling group) or —(N(R)C(O))ₚ—(CH₂)_{q}-(radiolabeling group);
r is 0 or 1;
n is 1, 2, 3, or 4;
m is 1, 2, 3, or 4;
p is 0 or 1;
q is 0, 1, 2, 3, 4, or 5; and
m1, m2, m3, and m4 are each independently 1, 2, 3, or 4.

In some embodiments, the compound of the present disclosure relates to a compound of Formula II:

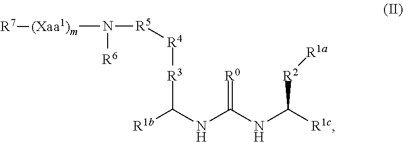

or is a salt or a solvate thereof; wherein:
R⁰ is O or S;
R^{1a} is —CO₂H, —SO₂H, —SO₃H, —PO₂H, —PO₃H₂, —OPO₃H₂, or —OSO₃H;
R^{1b} is —CO₂H, —SO₂H, —SO₃H, —PO₂H, or —PO₃H₂;
R^{1c} is —CO₂H, —SO₂H, —SO₃H, —PO₂H, or —PO₃H₂,
R² is —CH₂—, —(CH₂)₂—, —(CH₂)₃—, —CH₂CHF—, —CHFCH₂—, —CH₂OCH₂—, or —CH₂SCH₂—;
R³ is a linear or branched C₁-C₂₀ alkylenyl or alkenylenyl, or a linear or branched C₂-C₂₀ heteroalkylenyl or heteroalkenylenyl;
R⁴ is —O—, —S—, —S(O)—, —S(O)₂—, —NHC(O)—, —C(O)NH—,

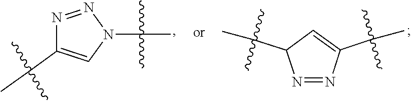

R⁵ is —(CH₂)₀₋₃CH(R¹⁰)(CH₂)₀₋₃—;
R¹⁰ is

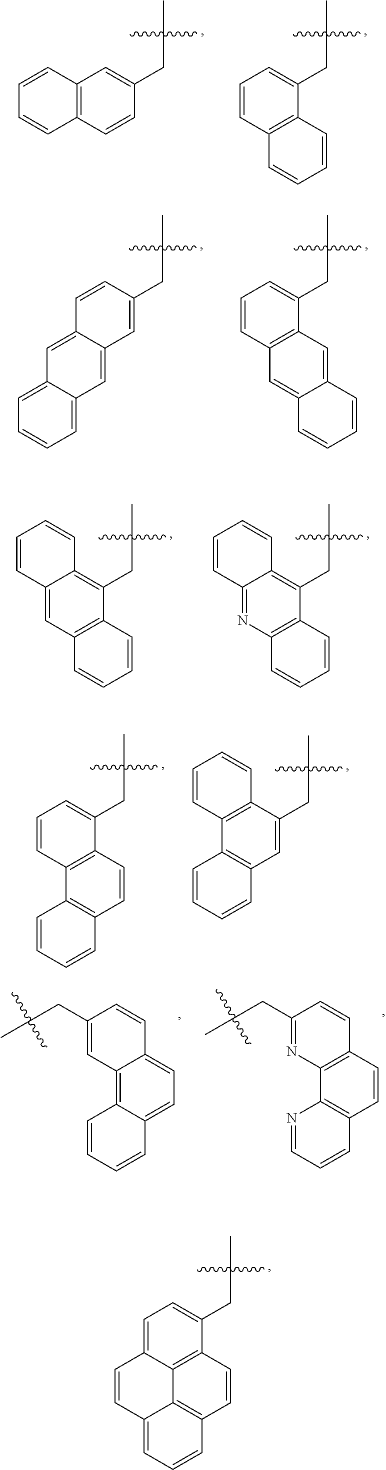

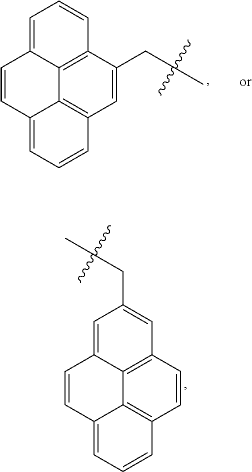 [SMILES: *Cc1cc2cccc3ccc4cccc1c4c32 ; *Cc1cc2ccc3cccc4ccc(c1)c2c34]

optionally modified with one, more than one, or a combination of: halogen, OMe, SMe, NH₂, NO₂, CN, OH, or additional endocyclic ring nitrogen atoms;
R⁶ is hydrogen, methyl or ethyl;
Xaa¹ is an amino acid of formula —N(R⁸)R⁹C(O)—,
each R⁸ is independently hydrogen or methyl;
each R⁹ is independently: -(a linear or branched C₁-C₁₀ alkylenyl or alkenylenyl)-cycloalkylene-, or -(a linear or branched C₂-C₁₀ heteroalkylenyl or heteroalkenylenyl)-cycloalkylene-;
R⁷ is

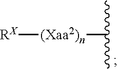

Xaa² is —N(R¹³)R¹⁴C(O)—;
each R¹³ is independently hydrogen or methyl;
each R¹⁴ is independently: a linear or branched C₁-C₂₀ alkylenyl or alkenylenyl, or a linear or branched C₂-C₂₀ heteroalkylenyl or heteroalkenylenyl;
wherein each R¹⁴ is optionally substituted with a carboxylic acid, a sulfonic acid, a sulfinic acid, a phosphoric acid, a hydroxyl, or an amide;
wherein each R¹⁴ is optionally substituted with one R^{X};
wherein at least one R¹⁴ is substituted with one R^{X};
R^{X} is

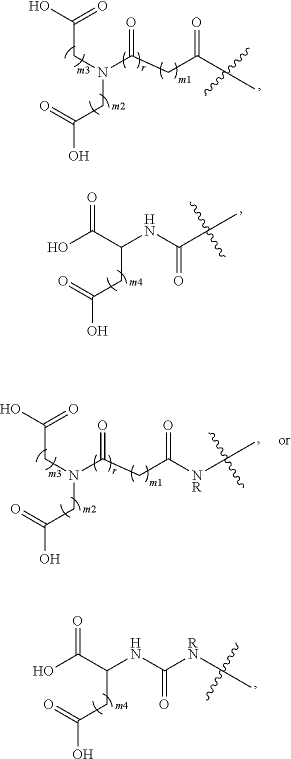

—C(O))ₚ(CH₂)_{q}-(radiolabeling group), or —(N(R)C(O))ₚ—(CH₂)_{q}-(radiolabeling group), wherein the radiolabeling group independently selected from: an aryl or heteroaryl substituted with a radioisotope or a prosthetic group comprising a trifluoroborate;
R is hydrogen or methyl;
wherein at least one R^{X} is

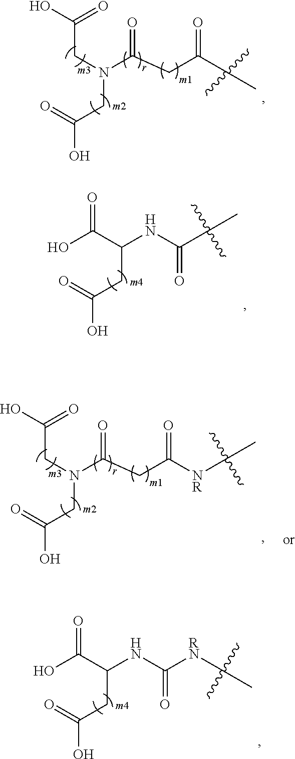

and at least one other R^{X} is —(C(O))ₚ—(CH₂)_{q}-(radiolabeling group) or —(N(R)C(O))ₚ—(CH₂)_{q}-(radiolabeling group);
r is 0 or 1
n is 1, 2, 3, or 4;
m is 1, 2, 3, or 4;
p is 0 or 1;
q is 0, 1, 2, 3, 4, or 5; and
m1, m2, m3, and m4 are each independently 1 or 2.

In some embodiments, the compound of the present disclosure relates to a compound of Formula III:

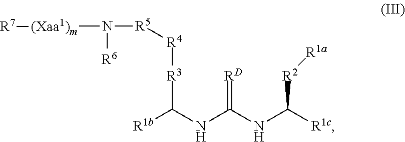

or is a salt or a solvate thereof; wherein:
R⁰ is O;
R^{1a} is —CO₂H;
R^{1b} is —CO₂H;
R^{1c} is —CO₂H;
R² is —(CH₂)₃—;
R³ is —CH₂—, —(CH₂)₂—, —(CH₂)₃, —(CH₂)₄—, —(CH₂)₅—, —CH₂—O—CH₂—, —CH₂—O—CH₂—CH₂—, —CH₂—CH₂—O—CH₂—, or —CH₂—S—CH₂—;
R⁴ is —NHC(O)— or —C(O)NH—;
R⁵ is —(CH₂)₀₋₃CH(R¹⁰)(CH₂)₀₋₃—, wherein R¹⁰ is selected from:

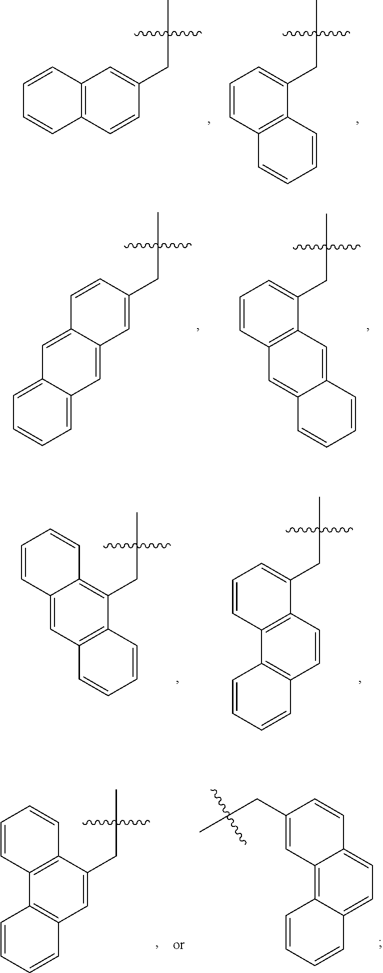

R⁶ is hydrogen or methyl;
Xaa¹ is an amino acid of formula —N(R⁸)R⁹C(O)—;
each R⁸ is independently hydrogen or methyl; and
each R⁹ is independently

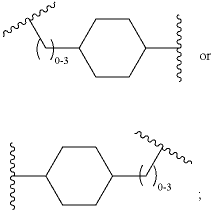

R⁷ is

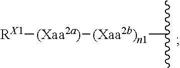

Xaa^{2a} is

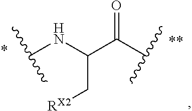

wherein * end connects to R^{X1} and ** end connects to Xaa²b;
Xaa^{2b} is *—N(R¹³)R¹⁴C(O)—**, wherein * end connects to Xaa^{2a};
each R¹³ is independently hydrogen or methyl;
each R¹⁴ is independently: a linear or branched C₁-C₆ alkylenyl or alkenylenyl, or a linear or branched C₂-C₆ heteroalkylenyl or heteroalkenylenyl; wherein each R¹⁴ is optionally substituted with a carboxylic acid, a sulfonic acid, a sulfinic acid, a phosphoric acid, a hydroxyl, or an amide;
R^{X1} is

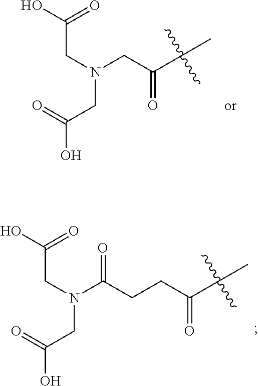 [SMILES: *C(=O)CN(CC(=O)O)CC(=O)O ; *C(=O)CCC(=O)N(CC(=O)O)CC(=O)O]

R^{X2} is —(N(R)C(O))ₚ(CH₂)_{q}(radiolabeling group), wherein the radiolabeling group independently selected from: an aryl or heteroaryl substituted with a radioisotope or a prosthetic group comprising a trifluoroborate;
R is hydrogen or methyl;
n1 is 0, 1, 2, or 3;
m is 1, 2, 3, or 4;
p is 0 or 1; and
q is 0, 1, 2, 3, 4, or 5.

In some embodiments, the compound of is a salt or solvate of Formula I, II, and/or III.

In some embodiments of the compounds of the invention, R⁰ is O. In other embodiments, R⁰ is S.

In some embodiments of the compounds of the invention, R^{1a} is —CO₂H, —SO₂H, —SO₃H, —PO₂H, —PO₃H₂, —OPO₃H₂, or —OSO₃H. In some embodiments, R^{2a} is —CO₂H, —SO₂H, —SO₃H, —PO₂H, or —PO₃H₂. In some embodiments, R^{3a} is —CO₂H, —SO₂H, —SO₃H, —PO₂H, or —PO₃H₂.

In some embodiments of the compounds of the invention, R^{1a} is —CO₂H. In some embodiments, R^{1b} is —CO₂H. In some embodiments, R^{1c} is —CO₂H. In some embodiments, R^{1a} and R^{1b} are each —CO₂H. In some embodiments, R^{1a} and R^{1c} are each —CO₂H. In some embodiments, R^{1b} and R^{1c} are each —CO₂H. In some embodiments, R^{1a}, R^{1b} and R^{1c} are each —CO₂H. In some embodiments, R^{1a}, R^{1b} and R^{1c} are anionic or metalated salts of the foregoing.

In some embodiments of the compounds of the invention, R² is —CH₂—. In some embodiments, R² is —CH(OH)—. In some embodiments, R² is —CHF—. In some embodiments, R² is —CF₂—. In some embodiments, R² is —CH(CH₃)—. In some embodiments, R² is —C(CH₃)₂—.

In some embodiments of the compounds of the invention, R² is —(CH₂)₂—. In some embodiments, R² is —CH₂CH(OH)—. In some embodiments, R² is —CH₂CHF—. In some embodiments, R² is —CHFCH₂—. In some embodiments, R² is —CF₂CH₂—. In some embodiments, R² is —CH₂CF₂—. In some embodiments, R² is —CH(OH)CH₂—. In some embodiments, R² is —CH(CH₃)CH₂—. In some embodiments, R² is —CH₂CH(CH₃)—. In some embodiments, R² is —C(CH₃)₂CH₂—. In some embodiments, R² is —CH₂C(CH₃)₂—.

In some embodiments of the compounds of the invention, R² is —CH₂—, —CH(OH)—, —CHF—, —CF₂—, —CH(CH₃)—, —C(CH₃)₂—, —(CH₂)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

In some embodiments of the compounds of the invention, R² is —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂CH(COOH)CH₂—, or —CH₂CH₂CH(COOH)—. In some embodiments, R² is —CH₂OCH₂— or —CH₂SCH₂—.

In some embodiments of the compounds of the invention, R² is —CH₂—, —CH(OH)—, —CHF—, —CF₂—, —CH(CH₃)—, —C(CH₃)₂—, —(CH₂)₂—, —CHFCH₂—, —CF₂CH₂—, —CH(OH)CH₂—, —CH(CH₃)CH₂—, —C(CH₃)₂CH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, or —C(O)—NH—C(CH₃)₂—.

In some embodiments of the compounds of the invention, R² is —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

In some embodiments of the compounds of the invention, R² is —CH₂CH(OH)—, —CH₂CHF—, —CH₂CH(CH₃)—, —CH₂CH(COOH)—, —CH₂CH(OH)CH₂—, —CH₂CH(F)CH₂—, or —CH₂CH(CH₃)CH₂—, wherein the second carbon in R² has R-configuration. In some embodiments, R² is —CH₂CH(OH)—, —CH₂CHF—, or —CH₂CH(CH₃)—, wherein the second carbon in R² has R-configuration. In some embodiments, R² is —CH₂CHF—, wherein the second carbon in R² has R-configuration.

In some embodiments of the compounds of the invention, R² is —CHOH—. In some embodiments, R² is —CH₂CHOH—. In some embodiments, R² is —CH₂CHOHCH₂—. In some embodiments, R² is —CH₂CHFCH₂. In some embodiments, R² is —(CH₂)₂CHOH—. In some embodiments, R² is —(CH₂)₂CHF—. In some embodiments, R² is —(CH₂)₃—. In some embodiments, R² is —CH₂OCH₂—. In some embodiments, R² is —CH₂SCH₂—.

In some embodiments of the compounds of the invention, R² is —CH₂CH(OH)CH₂—. In some embodiments, R² is —CH₂CHFCH₂—. In some embodiments, R² is —(CH₂)₂CH(OH)—. In some embodiments, R² is —CHFCH₂CH₂—. In some embodiments, R² is —CH(OH)CH₂CH₂—. In some embodiments, R² is —CH(CH₃)CH₂CH₂—. In some embodiments, R² is —CH₂CH(CH₃)CH₂—. In some embodiments, R² is —CH₂CH₂CH(CH₃)—. In some embodiments, R² is —C(CH₃)₂CH₂CH₂—. In some embodiments, R² is —CH₂C(CH₃)₂CH₂—. In some embodiments, R² is —CH₂CH₂C(CH₃)₂—. In some embodiments, R² is —CH(CH₃)—O—CH₂—. In some embodiments, R² is —C(CH₃)₂—O—CH₂—. In some embodiments, R² is —CH₂—O—CH(CH₃)—. In some embodiments, R² is —CH₂—O—C(CH₃)₂—. In some embodiments, R² is —CH₂—S(O)—CH₂—. In some embodiments, R² is —CH₂—S(O)₂—CH₂—. In some embodiments, R² is —CH(CH₃)—S—CH₂—. In some embodiments, R² is —C(CH₃)₂—S—CH₂—. In some embodiments, R² is —CH₂—S—CH(CH₃)—. In some embodiments, R² is —CH₂—S—C(CH₃)₂—. In some embodiments, R² is —CH(CH₃)—S(O)—CH₂—. In some embodiments, R² is —C(CH₃)₂—S(O)—CH₂—. In some embodiments, R² is —CH₂—S(O)—CH(CH₃)—. In some embodiments, R² is —CH₂—S(O)—C(CH₃)₂—. In some embodiments, R² is —CH(CH₃)—S(O)₂—CH₂—. In some embodiments, R² is —C(CH₃)₂—S(O)₂—CH₂—. In some embodiments, R² is —CH₂—S(O)₂—CH(CH₃)—. In some embodiments, R² is —CH₂—S(O)₂—C(CH₃)₂—. In some embodiments, R² is —CH₂—NH—C(O)—. In some embodiments, R² is —C(O)—NH—CH₂—. In some embodiments, R² is —C(O)—NH—CH(CH₃)—. In some embodiments, R² is —C(O)—NH—C(CH₃)₂—.

In some embodiments of the compounds of the invention, R² is —CH₂—, —(CH₂)₂—, —(CH₂)₃—, —CH₂CHF—, —CHFCH₂—, —CH₂OCH₂—, or —CH₂SCH₂—. In some embodiments, R² is —(CH₂)₂— or —(CH₂)₃—.

In some embodiments of the compounds of the invention, R^{1a} is —CO₂H and R² is —(CH₂)₂— or —(CH₂)₃—. In some embodiments, R^{1a} is —CO₂H and R² is —CH₂—. In some embodiments, R^{1a} is —CO₂H, R^{1b} is —CO₂H, R^{1c} is —CO₂H, and R² is —(CH₂)₂— or —(CH₂)₃—. In some embodiments, R^{1a} is —CO₂H and R² is —CH₂—.

In some embodiments, R³ is a linear or branched, cyclic or acyclic, and/or aromatic or non-aromatic C₁-C₁₀ alkylenyl, alkenylenyl, or alkynylenyl, or a linear or branched, cyclic or acyclic, and/or aromatic or non-aromatic C₂-C₁₀ heteroalkylenyl, heteroalkenylenyl, or heteroalkynylenyl. In some embodiments, R³ is a linear or branched, cyclic or acyclic, and/or aromatic or non-aromatic C₁-C₂₀ alkylenyl or alkenylenyl, or a linear or branched, cyclic or acyclic, and/or aromatic or non-aromatic C₂-C₂₀ heteroalkylenyl or heteroalkenylenyl. In some embodiments, R³ is a linear or branched, cyclic or acyclic, and/or aromatic or non-aromatic C₁-C₁₀ alkylenyl or alkenylenyl, or a linear or branched, cyclic or acyclic, and/or aromatic or non-aromatic C₂-C₁₀ heteroalkylenyl or heteroalkenylenyl. In some embodiments, R³ is a linear or branched, cyclic or acyclic, and/or aromatic or non-aromatic C₁-C₆ alkylenyl or alkenylenyl, or a linear or branched, cyclic or acyclic, and/or aromatic or non-aromatic C₂-C₆ heteroalkylenyl or heteroalkenylenyl.

In some embodiments of the compounds of the invention, R³ is a linear acyclic C₃-C₁₅ alkylenyl. In some embodiments, R³ is a linear acyclic C₃-C₁₅ heteroalkylenyl in which 1-5 carbons are replaced with N, S and/or O heteroatoms. In some embodiments, R³ is a linear acyclic saturated C₃-C₁₀ alkylenyl, optionally substituted with 1-5 amine, amide, oxo, hydroxyl, thiol, methyl or ethyl groups.

In some embodiments of the compounds of the invention, R³ is —(CH₂)₃₋₁₅—. In some embodiments, R³ is —(CH₂)₃₋₁₀—. In some embodiments, R³ is —(CH₂)₃₋₆—. In some embodiments, R³ is —CH₂—. In some embodiments, R³ is —(CH₂)₂—. In some embodiments, R³ is —(CH₂)₃—. In some embodiments, R³ is —(CH₂)₄—. In some embodiments, R³ is —(CH₂)₅—. In some embodiments, R³ is —CH₂—O—CH₂—. In some embodiments, R³ is —CH₂—S—CH₂—. In some embodiments, R³ is —CH═CH—. In some embodiments, R³ is —CH₂—C≡C—. In some embodiments, R³ is a linear C₃-C₅ alkenylenyl and/or alkynylenyl.

In some embodiments of the compounds of the invention, R³ is —CH₂—; —(CH₂)₂—; —(CH₂)₃; —(CH₂)₄—; —(CH₂)₅—; —CH₂—O—CH₂—; —CH₂—O—CH₂—CH₂—; —CH₂—CH₂—O—CH₂—; or —CH₂—S—CH₂—. In some embodiments, R³ is —(CH₂)₄—; —CH₂—O—CH₂—CH₂—; or —CH₂—CH₂—O—CH₂—. In some embodiments, R³ is —(CH₂)₄—. In some embodiments, R³ is —CH₂—O—CH₂—CH₂—. In some embodiments, R³ is —CH₂—CH₂—O—CH₂—. In some embodiments, R³ is —CH₂—O—CH₂—CH₂—**, where ** end connects to R⁴. In some embodiments, R³ is —CH₂—CH₂—O—CH₂—**, where ** end connects to R⁴. In some embodiments, R³ is —(CH₂)₄— or —CH₂—CH₂—CH₂—**, where ** end connects to R⁴.

In some embodiments of the compounds of the invention, R⁴ is —O—. In some embodiments, R⁴ is —S—. In some embodiments, R⁴ is —NHC(O)—. In some embodiments, R⁴ is —C(O)NH—. In some embodiments, R⁴ is

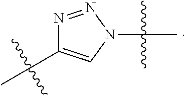 [SMILES: *c1cn(*)nn1]

In some embodiments, R⁴ is

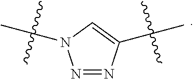 [SMILES: *c1cn(*)nn1]

In some embodiments, R⁴ is —S(O)—. In some embodiments, R⁴ is —S(O)—. In some embodiments, R⁴ is —S(O)—(NH)₂—C(O)—. In some embodiments, R⁴ is —(O)NH—. In some embodiments, R⁴ is —NH)(O)C—. In some embodiments, R⁴ is —NHC(O)NH—. In some embodiments, R⁴ is —C(S)NH. In some embodiments, R⁴ is —NHC(S)O—. In some embodiments, R⁴ is —NHC(S)NH—. In some embodiments, R⁴ is —NHC(O)C(O)NH—. In some embodiments, R⁴ is —S—S—. In some embodiments, R⁴ is —S—CH₂—S—. In some embodiments, R⁴ is —NH—NH—C(O)—. In some embodiments, R⁴ is or —C(O)—NH—NH—.

In some embodiments of the compounds of the invention, R⁴ is —O—, —S—, —S(O)—, —S(O)₂—, —NHC(O)—, —C(O)NH—,

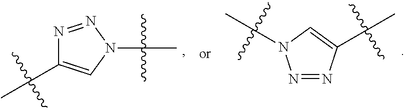 [SMILES: *c1cn(*)nn1 ; *c1cn(*)nn1]

In some embodiments of the compounds of the invention, R³ is —(CH₂)₃₋₁₅— and R⁴ is —NHC(O)—. In some embodiments, R³ is —(CH₂)₃₋₁₀— and R⁴ is —NHC(O)—. In some embodiments, R³ is —(CH₂)₃₋₆— and R⁴ is —NHC(O)—. In some embodiments, R³ is —(CH₂)₃₋₅— and R⁴ is —NHC(O)—. In some embodiments, R³ is —(CH₂)₄— and R⁴ is —NHC(O)—. In some embodiments, R³ is —(CH₂)₄— and R⁴ is —NHC(O)—**, where ** end connects to R⁵. In some embodiments of the compounds of the invention, R³ is —(CH₂)₃₋₁₅— and R⁴ is —C(O)NH—. In some embodiments, R³ is —(CH₂)₃₋₁₀— and R⁴ is —C(O)NH—. In some embodiments, R³ is —(CH₂)₃₋₆— and R⁴ is —C(O)NH—. In some embodiments, R³ is —(CH₂)₃₋₅— and R⁴ is —C(O)NH—. In some embodiments, R³ is —(CH₂)₄— and R⁴ is —C(O)NH—. In some embodiments, R³ is —(CH₂)₄— and R⁴ is —C(O)NH—**, where ** end connects to R⁵.

In some embodiments of the compounds of the invention, R⁵ is a linear or branched, cyclic or acyclic, and/or aromatic or non-aromatic C₁-C₃₀ alkylenyl or alkenylenyl, or is a linear or branched, cyclic or acyclic, and/or aromatic or non-aromatic C₂-C₃₀ heteroalkylenyl or heteroalkenylenyl. In some embodiments, R⁵ is a linear or branched, cyclic or acyclic, and/or aromatic or non-aromatic C₁-C₂₀ alkylenyl or alkenylenyl, or is a linear or branched, cyclic or acyclic, and/or aromatic or non-aromatic C₂-C₂₀ heteroalkylenyl or heteroalkenylenyl.

In some embodiments of the compounds of the invention, R⁵ is a C₁-C₆ alkylenyl substituted with C₅-C₂₀ aryl, C₅-C₂₀ arylalkyl, C₅-C₂₀ heteroaryl or C₅-C₂₀ heteroarylalkyl. In some embodiments, R⁵ is a C₁-C₃ alkylenyl substituted with C₁₀-C₂₀ aryl, C₁₀-C₂₀ arylalkyl, C₁₀-C₂₀ heteroaryl or C₁₀-C₂₀ heteroarylalkyl. In some embodiments, R⁵ is a C₁-C₃ alkylenyl substituted with C₁₀-C₁₆ aryl, C₁₀-C₁₆ arylalkyl, C₁₀-C₁₆ heteroaryl or C₁₀-C₁₆ heteroarylalkyl. In some embodiments, R⁵ is a C₁ alkylenyl substituted with C₁₀-C₁₆ aryl, C₁₀-C₁₆ arylalkyl, C₁₀-C₁₆ heteroaryl or C₁₀-C₁₆ heteroarylalkyl.

In some embodiments of the compounds of the invention, R⁵ is —(CH₂)₀₋₃CH(R¹⁰)(CH₂)₀₋₃—. In some embodiments, R⁵ is —(CH₂)₀₋₁CH(R¹⁰)(CH₂)₀₋₁—. In some embodiments, R⁵ is —CH(R¹⁰)—. In some embodiments, R⁵ is —CH₂CH(R¹⁰)—. In some embodiments, R⁵ is —CH(R¹⁰)CH₂—. In some embodiments, R⁵ is —CH(R¹⁰)—.

In some embodiments of the compounds of the invention, R¹⁰ is a linear or branched, cyclic or acyclic, and/or aromatic or non-aromatic C₂-C₁₉ alkyl, alkenyl or alkynyl; a linear or branched, cyclic or acyclic, and/or aromatic or non-aromatic C₂-C₁₉ heteroalkyl, heteroalkenyl or heteroalkynyl having only 1-3 heteroatoms.

In some embodiments of the compounds of the invention, R¹⁰ is C₅-C₂₀ aryl, C₅-C₂₀ arylalkyl, C₅-C₂₀ heteroaryl or C₅-C₂₀ heteroarylalkyl. In some embodiments, R¹⁰ is C₁₀-C₂₀ aryl, C₁₀-C₂₀ arylalkyl, C₁₀-C₂₀ heteroaryl or C₁₀-C₂₀ heteroarylalkyl. In some embodiments, R¹⁰ is C₁₀-C₁₆ aryl, C₁₀-C₁₆ arylalkyl, C₁₀-C₁₆ heteroaryl or C₁₀-C₁₆ heteroarylalkyl.

In some embodiments of the compounds of the invention, R¹⁰ is —CH₂R²³, in which R²³ is an optionally substituted C₄-C₁₆ aromatic ring or partially or fully aromatic fused ring system, wherein 0-3 carbons in the aromatic ring or the partially or fully aromatic fused ring system are replaced with N, S and/or O heteroatoms, and wherein the optional substitutions are selected from OH, NH₂, NO₂, halogen, C₁-C₆ alkyl, and/or C₁-C₆ alkoxyl groups.

In some embodiments of the compounds of the invention, R¹⁰ is

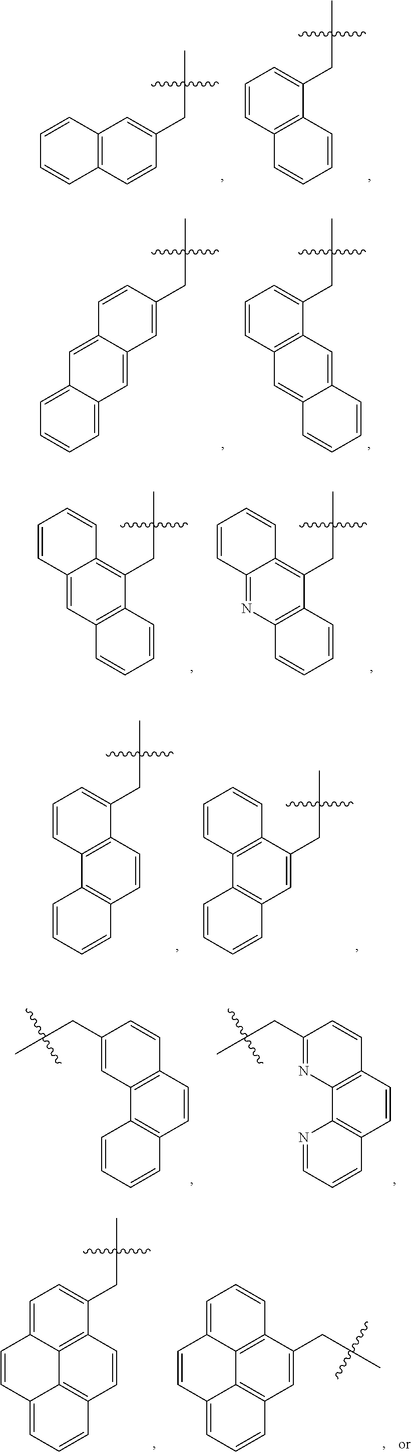

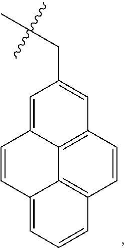 [SMILES: *Cc1cc2ccc3cccc4ccc(c1)c2c34]

optionally modified with one, more than one, or a combination of: halogen, OMe, SMe, NH₂, NO₂, CN, OH, or one or more additional endocyclic ring nitrogen atoms.

In some embodiments of the compounds of the invention, R¹⁰ is an alkenyl containing either a C₆-C₁₆ aryl or C₆-C₁₆ heteroaryl having 1-3 heteroatoms independently selected from N, S and/or O. In some embodiments, the C₆-C₁₆ aryl is benzyl. In some embodiments, the C₆-C₁₆ heteroaryl is benzyloxyl or benzylthio.

In some embodiments, R¹⁰ is:

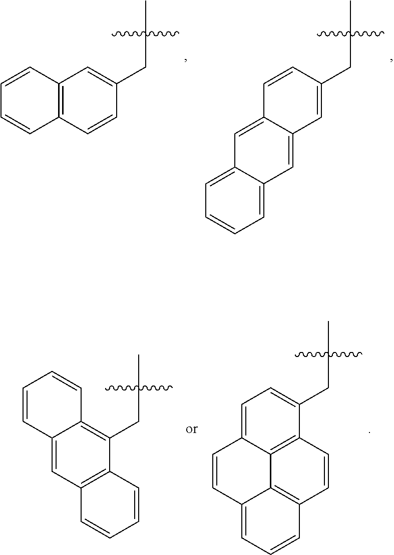 [SMILES: *Cc1ccc2ccccc2c1 ; *Cc1ccc2cc3ccccc3cc2c1 ; *Cc1c2ccccc2cc2ccccc12 ; *Cc1ccc2ccc3cccc4ccc1c2c34]

In some embodiments, R¹⁰ is

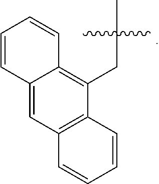 [SMILES: *Cc1c2ccccc2cc2ccccc12]

In some embodiments, R¹⁰ is

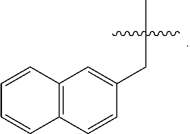 [SMILES: *Cc1ccc2ccccc2c1]

In some embodiments, R¹⁰ is

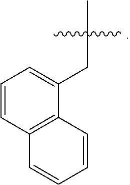 [SMILES: *Cc1cccc2ccccc12]

In some embodiments, R¹⁰ is

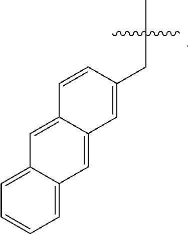 [SMILES: *Cc1ccc2cc3ccccc3cc2c1]

In some embodiments, R¹⁰ is

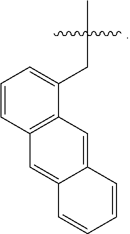 [SMILES: *Cc1cccc2cc3ccccc3cc12]

In some embodiments, R¹⁰ is

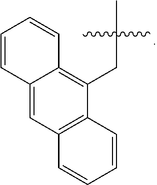 [SMILES: *Cc1c2ccccc2cc2ccccc12]

In some embodiments, R¹⁰ is

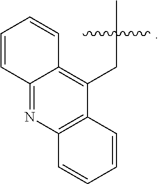 [SMILES: *Cc1c2ccccc2nc2ccccc12]

In some embodiments, R¹⁰ is

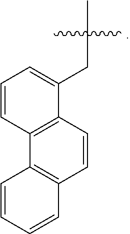 [SMILES: *Cc1cccc2c1ccc1ccccc12]

In some embodiments, R¹⁰ is

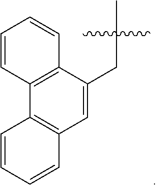 [SMILES: *Cc1cc2ccccc2c2ccccc12]

In some embodiments, R¹⁰ is

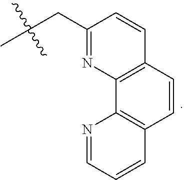 [SMILES: *Cc1ccc2ccc3cccnc3c2n1]

In some embodiments, R¹⁰ is

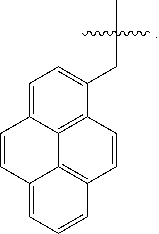 [SMILES: *Cc1ccc2ccc3cccc4ccc1c2c34]

In some embodiments, R¹⁰ is

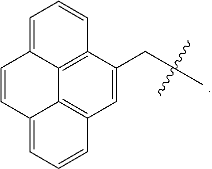 [SMILES: *Cc1cc2cccc3ccc4cccc1c4c32]

In some embodiments, R¹⁰ is

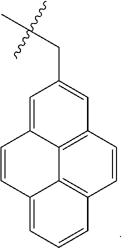 [SMILES: *Cc1cc2ccc3cccc4ccc(c1)c2c34]

In some embodiments, R¹⁰ is

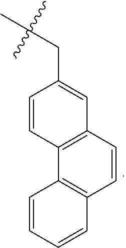 [SMILES: *Cc1ccc2c(ccc3ccccc32)c1]

In some embodiments, R¹⁰ is

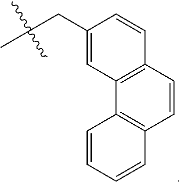 [SMILES: *Cc1ccc2ccc3ccccc3c2c1]

In some embodiments, R¹⁰ is

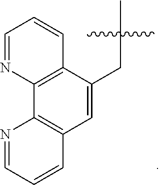 [SMILES: *Cc1cc2cccnc2c2ncccc12]

In some embodiments, R¹⁰ is

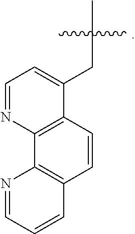 [SMILES: *Cc1ccnc2c1ccc1cccnc12]

In some embodiments, R¹⁰ is:

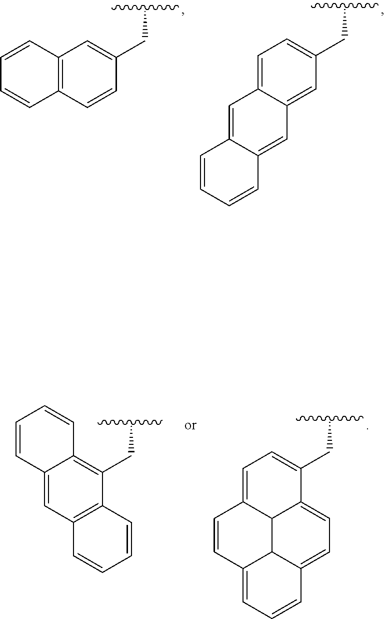

In some embodiments, R¹⁰ is

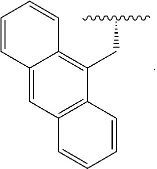

In some embodiments of the compounds of the invention, R⁵ is —CH(R¹⁰)— wherein R¹⁰ is

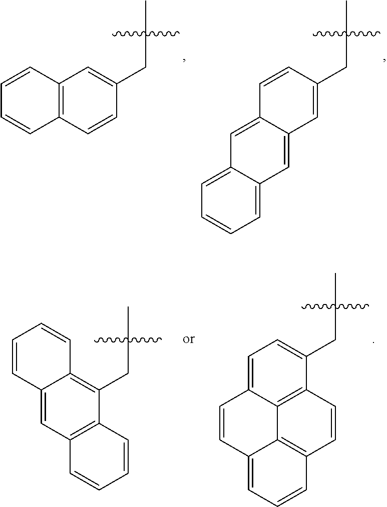 [SMILES: *Cc1ccc2ccccc2c1 ; *Cc1ccc2cc3ccccc3cc2c1 ; *Cc1c2ccccc2cc2ccccc12 ; *Cc1ccc2ccc3cccc4ccc1c2c34]

In some embodiments, R⁵ is —CH(R¹⁰)— wherein R¹⁰ is

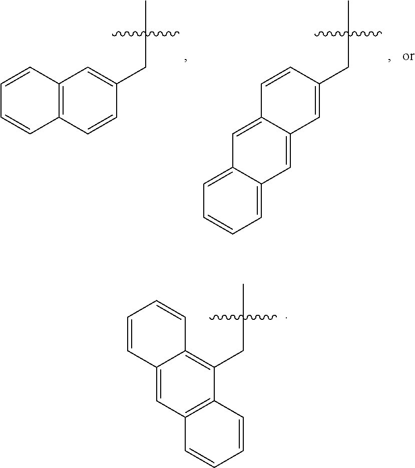 [SMILES: *Cc1ccc2ccccc2c1 ; *Cc1ccc2cc3ccccc3cc2c1 ; *Cc1c2ccccc2cc2ccccc12]

In some embodiments, R⁵ is —CH(R¹⁰)— wherein R¹⁰ is

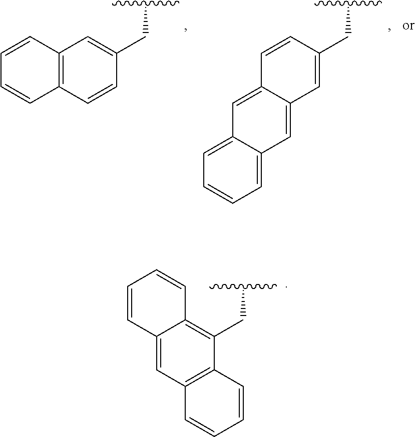

In some embodiments of the compounds of the invention, R⁵ is —CH(R¹⁰)— and R¹⁰ is

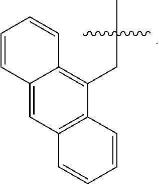 [SMILES: *Cc1c2ccccc2cc2ccccc12]

In some embodiments, R⁵ is —CH(R¹⁰)— and R¹⁰ is

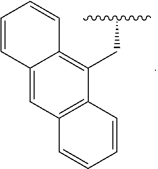

In some embodiments of the compounds of the invention, R⁵ is —(CH₂)₀₋₃CH(R¹⁰)(CH₂)₀₋₃— and R¹⁰ is —(CH₂)₅CH₃. In some embodiments, R⁵ is —CH(R¹⁰)— and R¹⁰ is —(CH₂)₅CH₃. In some embodiments, R⁵ is —(CH₂)₀₋₃CH(R¹⁰)(CH₂)₀₋₃—.

In some embodiments of the compounds of the invention, R¹⁰ is —CH²—R²³. In some embodiments, R²³ is phenyl substituted with 1 or 2 iodo groups and optionally further substituted with 1 oxy group. In some embodiments, R⁵ is —(CH₂)₀₋₃CH(R¹⁰)(CH₂)₀₋₃— wherein R¹⁰ is —CH₂R²³ and R²³ is phenyl substituted with 1 or 2 iodo groups and optionally further substituted with 1 oxy group. In some embodiments, R²³ is

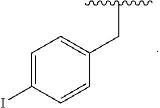 [SMILES: *Cc1ccc(I)cc1]

In some embodiments, R²³ is

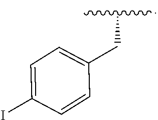

In some embodiments, R²³ is

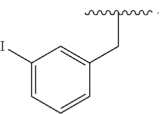 [SMILES: *Cc1cccc(I)c1]

In some embodiments, R²³ is

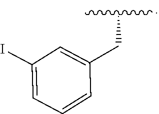

In some embodiments, R²³ is

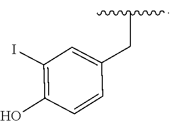 [SMILES: *Cc1ccc(O)c(I)c1]

In some embodiments, R²³ is

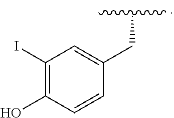

In some embodiments, R²³ is

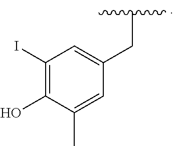 [SMILES: *Cc1cc(C)c(O)c(I)c1]

In some embodiments, R²³ is

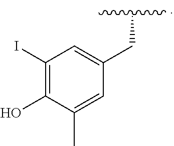

In some embodiments of the compounds of the invention, R⁶ is hydrogen. In some embodiments, R⁶ is methyl. In some embodiments, R⁶ is ethyl.

In some embodiments of the compounds of the invention, Xaa¹ is an amino acid of formula —N(R⁸)R⁹C(O)—.

In some embodiments of the compounds of the invention, m is 1. In some embodiments, m is 2 and each Xaa¹ may be the same or different. In some embodiments, m is 3 and each Xaa¹ may be the same, different or a combination thereof. In some embodiments, m is 4 and each Xaa¹ may be the same, different or a combination thereof. In some embodiments, each Xaa¹ is independently selected from proteinogenic amino acids and the non-proteinogenic amino acids listed in Table 1, wherein each peptide backbone amino group is optionally methylated.

In some embodiments of the compounds of the invention, each R⁸ is independently hydrogen or methyl. In some embodiments, at least one R⁸ is methyl.

In some embodiments of the compounds of the invention, R⁹ is a linear or branched, cyclic or acyclic, and/or aromatic or non-aromatic C₁-C₃₀ alkylenyl or alkenylenyl, or is a linear or branched, cyclic or acyclic, and/or aromatic or non-aromatic C₂-C₃₀ heteroalkylenyl or heteroalkenylenyl. In some embodiments, R⁹ is a linear or branched, cyclic or acyclic, and/or aromatic or non-aromatic C₁-C₂₀ alkylenyl or alkenylenyl, or is a linear or branched, cyclic or acyclic, and/or aromatic or non-aromatic C₂-C₂₀ heteroalkylenyl or heteroalkenylenyl.

In some embodiments of the compounds of the invention, R⁹ is a C₁-C₆ alkylenyl substituted with C₅-C₂₀ aryl, C₅-C₂₀ arylalkyl, C₅-C₂₀ heteroaryl or C₅-C₂₀ heteroarylalkyl. In some embodiments, R⁹ is a C₁-C₃ alkylenyl substituted with C₁₀-C₂₀ aryl, C₁₀-C₂₀ arylalkyl, C₁₀-C₂₀ heteroaryl or C₁₀-C₂₀ heteroarylalkyl. In some embodiments, R⁹ is a C₁-C₃ alkylenyl substituted with C₁₀-C₁₆ aryl, C₁₀-C₁₆ arylalkyl, C₁₀-C₁₆ heteroaryl or C₁₀-C₁₆ heteroarylalkyl. In some embodiments, R⁹ is a C₁ alkylenyl substituted with C₁₀-C₁₆ aryl, C₁₀-C₁₆ arylalkyl, C₁₀-C₁₆ heteroaryl or C₁₀-C₁₆ heteroarylalkyl.

In some embodiments of the compounds of the invention, R⁹ is —(CH₂)₀₋₃CH(R¹⁰)(CH₂)₀₋₃—. In some embodiments, R⁹ is —(CH₂)₀₋₁CH(R¹⁰)(CH₂)₀₋₁—. In some embodiments, R⁹ is —CH(R¹⁰)—. In some embodiments, R⁹ is —CH₂CH(R¹⁰)—. In some embodiments, R⁹ is —CH(R¹⁰)CH₂—. In some embodiments, R⁹ is —CH(R¹⁰)—.

In some embodiments of the compounds of the invention, R⁹ is —(C₁-C₆ alkylenyl)-(C₃-C₁₀ cycloalkylenyl)-, —(C₁-C₆ alkylenyl)-(C₃-C₁₀ cycloalkylenyl)-(C₁-C₆ alkylenyl)-, —(C₁-C₆ alkylenyl)-(C₆-C₁₂ arylene)-, or —(C₁-C₆ alkylenyl)-(C₆-C₁₂ arylene)-(C₁-C₆ alkylenyl)-. In some embodiments, R⁹ is —(C₁-C₃ alkylenyl)-(C₃-C₁₀ cycloalkylenyl)-, —(C₁-C₃ alkylenyl)-(C₃-C₁₀ cycloalkylenyl)-(C₁-C₃ alkylenyl)-, —(C₁-C₃ alkylenyl)-(C₆-C₁₂ arylene)-, or —(C₁-C₃ alkylenyl)-(C₆-C₁₂ arylene)-(C₁-C₃ alkylenyl)-. In some embodiments, R⁹ is —(C₁-C₃ alkylenyl)-(C₃-C₁₀ cycloalkylenyl)- or —(C₁-C₃ alkylenyl)-(C₆-C₁₂ arylene)-. In some embodiments, R⁹ is —(C₁-C₃ alkylenyl)-(C₃-C₁₀ cycloalkylenyl)-.

In some embodiments of the compounds of the invention, R⁹ is

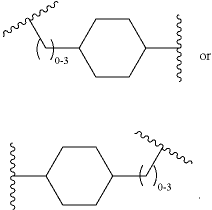

In some embodiments, R⁹ is or

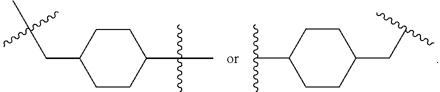 [SMILES: *CC1CCC(*)CC1 ; *CC1CCC(*)CC1]

In some embodiments of the compounds of the invention, at least one R⁹ is R²⁴—R²⁵—R²⁶, wherein R²⁴—R²⁵—R²⁶ are independently selected from: —(CH₂)₀₋₃—; C₃-C₈ cycloalkylene in which 0-3 carbons are replaced with N, S or O heteroatoms, and optionally substituted with one or more OH, NH₂, NO₂, halogen, C₁-C₆ alkyl and/or C₁-C₆ alkoxyl groups; and C₄-C₁₆ arylene in which 0-3 carbons are replaced with N, S or O heteroatoms, and optionally substituted with one or more OH, NH₂, NO₂, halogen, C₁-C₆ alkyl and/or C₁-C₆ alkoxyl groups. In some embodiments, (Xaa¹)₁₋₄ is (Xaa¹)₀₋₃NHR²⁷C(O), wherein R²⁷ is

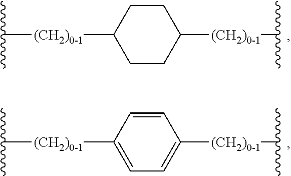 [SMILES: *CC1CCC(C*)CC1 ; *Cc1ccc(C*)cc1]

or

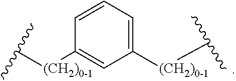 [SMILES: *Cc1cccc(C*)c1]

In some embodiments, at least one R⁹ is

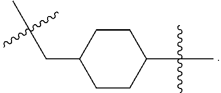 [SMILES: *CC1CCC(*)CC1]

In some embodiments, at least one R⁹ is

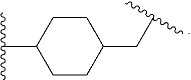 [SMILES: *CC1CCC(*)CC1]

In some embodiments, at least one R⁹ is

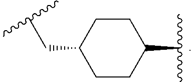

In some embodiments, at least one R⁸ is hydrogen. In some embodiments, all R⁸ are hydrogen. In some embodiments, at least one Xaa¹ is a tranexamic acid residue. In some embodiments, (Xaa¹)₁₋₄ consists of a single tranexamic acid residue.

In some embodiments, at least one R⁹ or R⁵ is

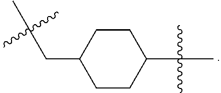 [SMILES: *CC1CCC(*)CC1]

In some embodiments, at least one R⁹ or R⁵ is

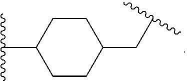 [SMILES: *CC1CCC(*)CC1]

In some embodiments, at least one R⁹ or R⁵ is

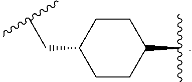

In some embodiments, R⁵ is

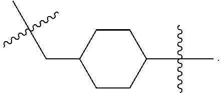 [SMILES: *CC1CCC(*)CC1]

In some embodiments, R⁵ is

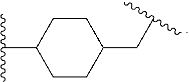 [SMILES: *CC1CCC(*)CC1]

In some embodiments, R⁵ is

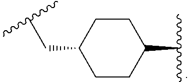

In some embodiments, R³ is —(CH₂)₄— and —(Xaa¹)ₘN(R⁶)R⁵R⁴— is

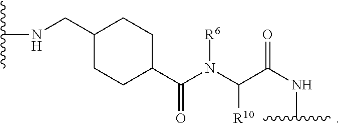

In some embodiments, R¹⁰ is

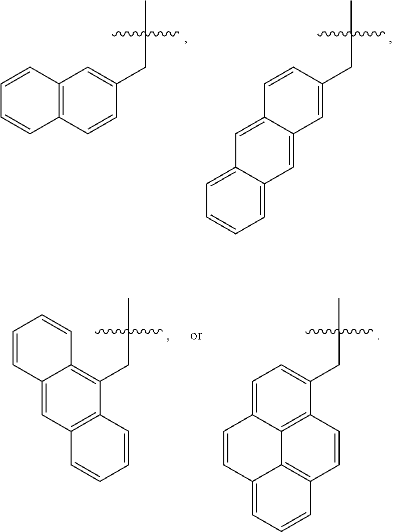 [SMILES: *Cc1ccc2ccccc2c1 ; *Cc1ccc2cc3ccccc3cc2c1 ; *Cc1c2ccccc2cc2ccccc12 ; *Cc1ccc2ccc3cccc4ccc1c2c34]

In some embodiments, R³ is —CH₂—CH₂—O—CH₂— and —(Xaa¹)ₘN(R⁶)R⁵R⁴— is

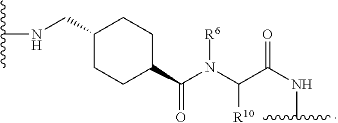

In some embodiments, R¹⁰ is

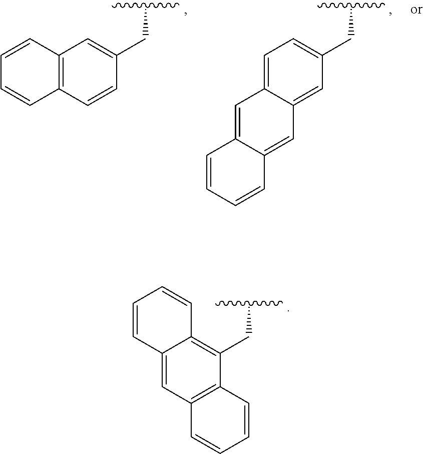

R⁷ may include a radiolabeling group optionally spaced apart using an amino acid or peptide linker and a nitriloacetic acid or derivative thereof.

In some embodiments of the compounds of the invention, R⁷ is

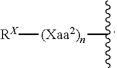

In some embodiments of the compounds of the invention, Xaa² is —N(R¹³)R¹⁴C(O)—.

In some embodiments of the compounds of the invention, n is 1. In some embodiments, n is 2 and each Xaa² may be the same or different. In some embodiments, n is 3 and each Xaa² may be the same, different or a combination thereof. In some embodiments, n is 4 and each Xaa² may be the same, different or a combination thereof. In some embodiments, each Xaa² is independently selected from proteinogenic amino acids and the non-proteinogenic amino acids listed in Table 1, wherein each peptide backbone amino group is optionally methylated.

In some embodiments of the compounds of the invention, each R¹³ is independently hydrogen or methyl. In some embodiments, at least one R¹³ is methyl.

In some embodiments of the compounds of the invention, each R¹⁴ is independently: a linear or branched, cyclic or acyclic, and/or aromatic or non-aromatic C₁-C₁₅ alkylenyl, alkenylenyl or alkynylenyl; or a linear or branched, cyclic or acyclic, and/or aromatic or non-aromatic C₂-C₁₅ heteroalkylenyl, heteroalkenylenyl or heteroalkynylenyl; wherein each R¹⁴ is optionally substituted with a carboxylic acid, a sulfonic acid, a sulfinic acid, a phosphoric acid, a hydroxyl, or an amide; each R¹⁴ is optionally substituted with one R^{X}; and at least one R¹⁴ is substituted with one R^{X}. In some embodiments, each R¹⁴ is independently: a linear or branched, cyclic or acyclic, and/or aromatic or non-aromatic C₁-C₁₀ alkylenyl, alkenylenyl or alkynylenyl; or a linear or branched, cyclic or acyclic, and/or aromatic or non-aromatic C₂-C₁₀ heteroalkylenyl, heteroalkenylenyl or heteroalkynylenyl; wherein each R¹⁴ is optionally substituted with a carboxylic acid, a sulfonic acid, a sulfinic acid, a phosphoric acid, a hydroxyl, or an amide; each R¹⁴ is optionally substituted with one R^{X}; and at least one R¹⁴ is substituted with one R^{X}. In some embodiments, each R¹⁴ is independently: a linear or branched, cyclic or acyclic, and/or aromatic or non-aromatic C₁-C₆ alkylenyl, alkenylenyl or alkynylenyl; or a linear or branched, cyclic or acyclic, and/or aromatic or non-aromatic C₂-C₆ heteroalkylenyl, heteroalkenylenyl or heteroalkynylenyl; wherein each R¹⁴ is optionally substituted with a carboxylic acid, a sulfonic acid, a sulfinic acid, a phosphoric acid, a hydroxyl, or an amide; each R¹⁴ is optionally substituted with one R^{X}; and at least one R¹⁴ is substituted with one R^{X}.

In some embodiments of the compounds of the invention, each R¹⁴ is independently a linear or branched, acyclic C₁-C₁₅ alkylenyl. In some embodiments, each R¹⁴ is independently a linear or branched, acyclic C₁-C₁₀ alkylenyl. In some embodiments, each R¹⁴ is independently a linear or branched, acyclic C₁-C₆ alkylenyl.

In some embodiments of the compounds of the invention, each R¹⁴ is optionally substituted with one R^{X} and further optionally substituted with a carboxylic acid, a sulfonic acid, a sulfinic acid, a phosphoric acid, or any functional group found in an amino acid side chain. In some embodiments of the compounds of the invention, each R¹⁴ is optionally substituted with one R^{X} and further optionally substituted with a carboxylic acid, a sulfonic acid, a sulfinic acid, a phosphoric acid, or any functional group found in a natural or non-natural amino acid side chain. In some embodiments of the compounds of the invention, each R¹⁴ is optionally substituted with one R^{X} and further optionally substituted with a carboxylic acid, a sulfonic acid, a sulfinic acid, a phosphoric acid, a hydroxyl, an amide, an amine, hydrazine, hydrazide, thiohydrazide, urea, thiourea, guanidine, imidazole, pyrrolidine, or methylthiol. In some embodiments of the compounds of the invention, each R¹⁴ is optionally substituted with one R^{X} and further optionally substituted with a carboxylic acid, a sulfonic acid, a sulfinic acid, a phosphoric acid, a hydroxyl, or an amide.

In some embodiments, —N(R¹³)R¹⁴C(O)— is an amino acid residue. In some embodiments, —N(R¹³)R¹⁴C(O)— is a natural amino acid residue or a non-natural amino acid residue.

In some embodiments of the compounds of the invention, each Xaa² is independently selected from:

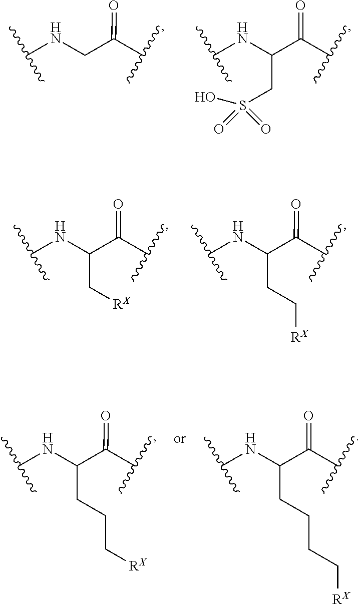

In some embodiments, each Xaa² is independently selected from:

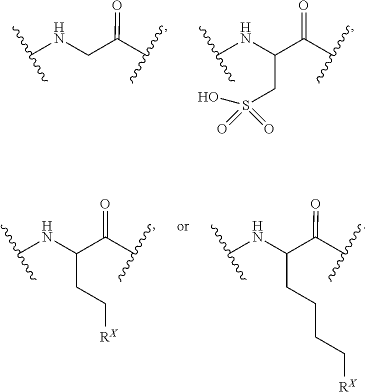

In some embodiments of the compounds of the invention, n is 1 and Xaa² is

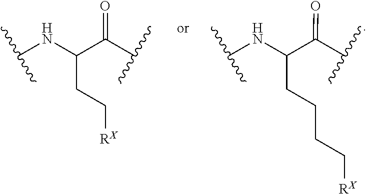

In some embodiments, n is 1 and Xaa² is

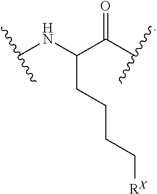

In some embodiments, n is 2 and each Xaa² is independently selected from:

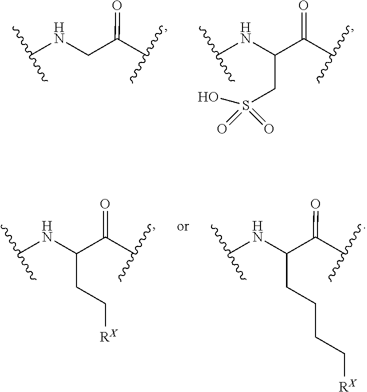

In some embodiments of the compounds of the invention, n is 3 and each Xaa² is independently selected from:

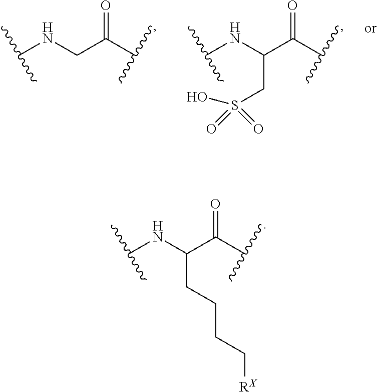

In some embodiments of the compounds of the invention, R⁷ is

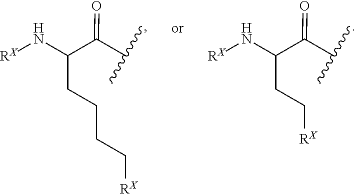

In some embodiments of the compounds of the invention, R^{X} is

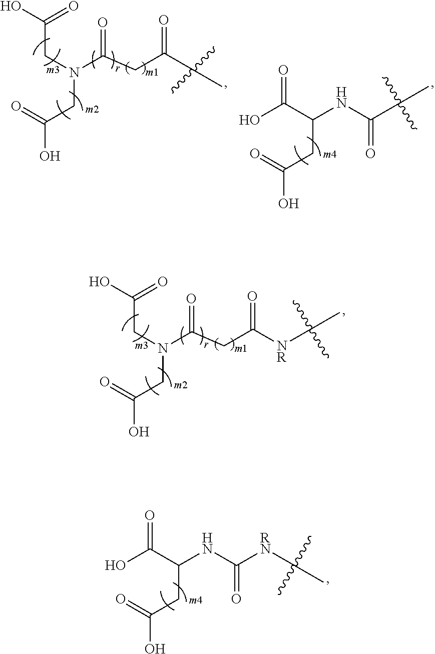

—(C(O))ₚ—(CH₂)_{q}-(radiolabeling group), or —(N(R)C(O))ₚ—(CH₂)_{q}-(radiolabeling group), wherein the radiolabeling group is independently selected from: an aryl or heteroaryl substituted with a radioisotope or a prosthetic group comprising a trifluoroborate, wherein r is 0 or 1; and wherein m1, m2, m3 and m4 are each independently 1, 2, 3, or 4.

In some embodiments, R^{X} can form bonds to the N-terminus of the N-terminal Xaa² or an amino acid group of Xaa² capable of forming an amide bond (e.g., a side chain of an alpha amino acid). An example of a Xaa² sidechain capable of forming an amide bond with R^{X} is an amino group. Non-limiting examples of amino acid residues capable of forming an amide with R^{X} include Lys, Orn, Dab, Dap, Arg, homo-Arg, and the like. In some embodiments, R^{X} forms a bond to the N-terminus of the N-terminal Xaa². In some embodiments, R^{X} may bond to different Xaa² side chains or other functional groups.

In some embodiments, at least one R^{X} is

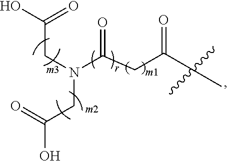

wherein r is 0 or 1; and wherein m1, m2, and m3 are each independently 1, 2, 3, or 4.

In some embodiments, at least one R^{X} is

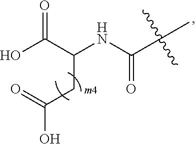

wherein m4 is 1, 2, 3, or 4.

In some embodiments, at least one R^{X} is

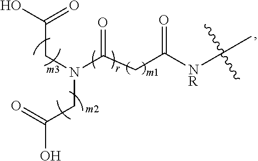

wherein r is 0 or 1; and wherein m1, m2, and m3 are each independently 1, 2, 3, or 4.

In some embodiments, at least one R^{X} is

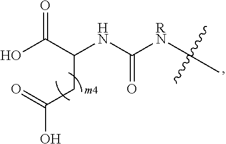

wherein m4 is 1, 2, 3, or 4.

In some embodiments, the radioisotope is a radiohalogen. The term “radiohalogen” refers to a radioactive isotope of a halogen. Thus, the term “radiohalogen: includes radioactive isotopes of F, Cl, Br, and I, such as ¹⁸fluorine, ⁷⁶bromine, ¹²³iodine, ¹²⁴iodine, and ¹³¹iodine. In one embodiment of the invention, a radiohalogen is ¹⁸fluorine, ¹²³iodine, or ¹²⁴iodine.

In some embodiments, one or more R^{X} may comprise an aryl group substituted with a radioisotope. In some embodiments, one or more R^{X} is

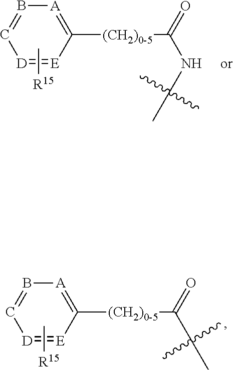

wherein A, B, C, D and E are independently CH or N, and R¹⁵ is a radioisotope.

In some embodiments, one or more R^{X} may comprise an aryl group substituted with a radioisotope. In some embodiments, one or more R^{X} is

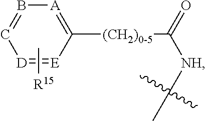

wherein A, B, C, D and E are independently CH or N, and R¹⁵ is a radioisotope. In some embodiments, one or more R^{X} is

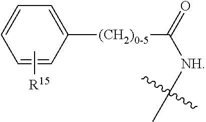

In some embodiments, one or more R^{X} is

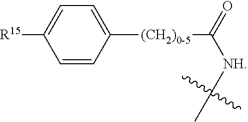

In some embodiments, one or more R^{X} is

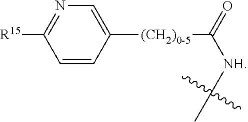

In some embodiments, one or more R^{X} is

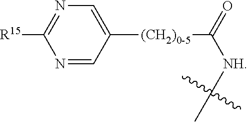

In some embodiments, one or more R^{X} is

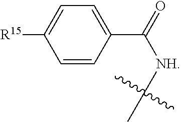

In some embodiments, one or more R^{X} is

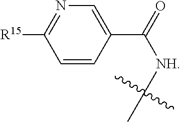

In some embodiments, one or more R^{X} is

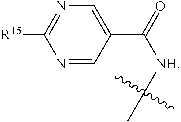

In some embodiments, one or more R^{X} is

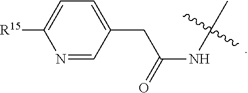

In some of these embodiments, R¹⁵ is independently ²¹¹At, ¹³¹I, ¹²⁴I, ¹²³I, ⁷⁷Br or ¹⁸F. In some of these embodiments, R¹⁵ is a radioisotope. In some of these embodiments, R¹⁵ is ¹⁸F.

In some embodiments, one or more R^{X} may comprise an aryl group substituted with a radioisotope. In some embodiments, one or more R^{X} is

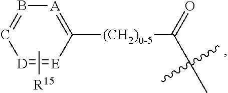

wherein A, B, C, D and E are independently CH or N, and R¹⁵ is a radioisotope. In some embodiments, one or more R^{X} is

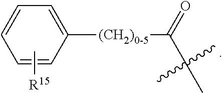

In some embodiments, one or more R^{X} is

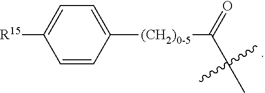

In some embodiments, one or more R^{X} is

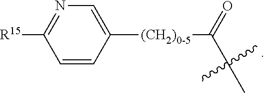

In some embodiments, one or more R^{X} is

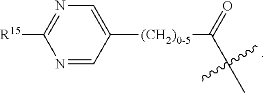

In some embodiments, one or more R^{X} is

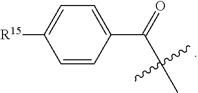

In some embodiments, one or more R^{X} is

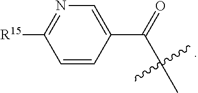

In some embodiments, one or more R^{X} is

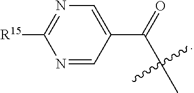

In some embodiments, one or more R^{X} is

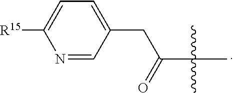

In some of these embodiments, R¹⁵ is independently ²¹¹At, ¹³¹I, ¹²⁴I, ¹²³I, ⁷⁷Br or ¹⁸F. In some of these embodiments, R¹⁵ is a radioisotope. In some of these embodiments, R¹⁵ is ¹⁸F.

In some embodiments, one or more R^{X} may comprise a prosthetic group containing a trifluoroborate (BF₃), capable of ¹⁸F/¹⁹F exchange radiolabeling.

In some embodiments of the compounds of the invention, one or more R^{X} is —(N(R)C(O))ₚ—(CH₂)_{q}—(R¹⁶R¹⁷BF₃); each R¹⁶ is independently absent,

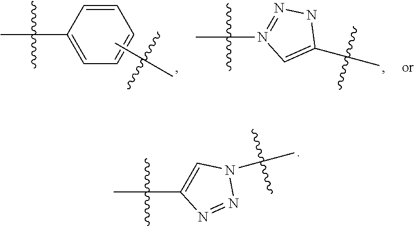

In some embodiment, R^{X} is —(N(R)C(O))ₚ—(CH₂)_{q}—(R¹⁶R¹⁷BF₃); each R¹⁶ is independently absent,

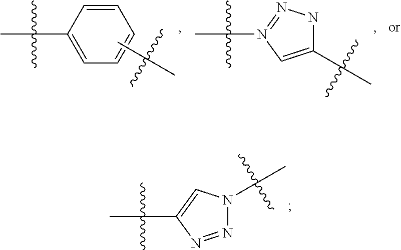

each R¹⁷BF₃ is independently selected from Table 2 (below), Table 3 (below), or

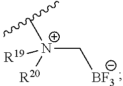

and R¹⁹ and R²⁰ are independently C₁-C₅ linear or branched alkyl. For Tables 2 and 3, the R in the pyridine substituted with —OR, —SR, —NR—, —NHR or —NR₂ groups is C₁-C₅ branched or linear alkyl. In some embodiments, one or more —R¹⁷BF₃ is independently selected from one or a combination of those listed in Table 2. In some embodiments, one or more —R¹⁷BF₃ is independently selected from one or a combination of those listed in Table 3. In some embodiments, one fluorine is ¹⁸F. In some embodiments, all three fluorines are ¹⁹F.

In some embodiments of the compounds of the invention, one or more R^{X} is —N(R)C(O))ₚ—(CH₂)_{q}—(R¹⁶R¹⁷BF₃); each R¹⁶ is independently absent,

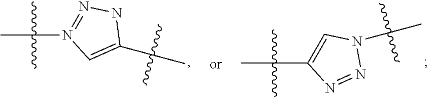

and —R¹⁷BF₃ is

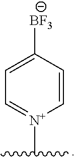

TABLE 2
Exemplary R¹⁷BF₃ groups.

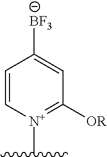

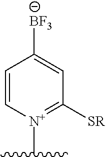

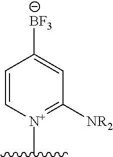

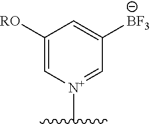

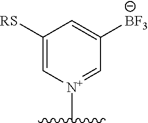

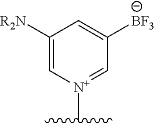

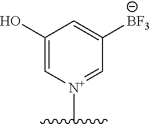

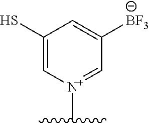

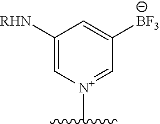

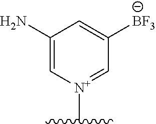

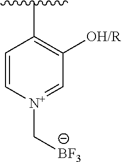

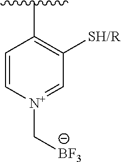

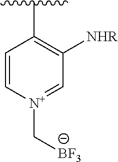

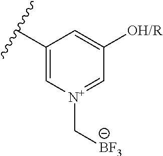

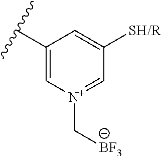

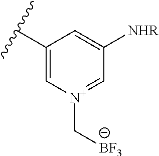

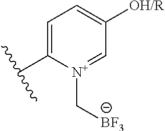

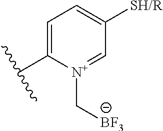

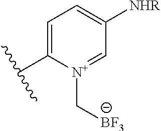

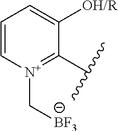

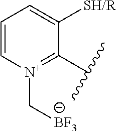

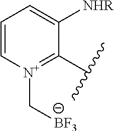

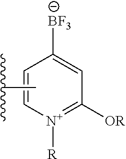

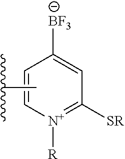

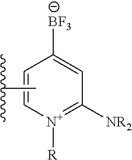

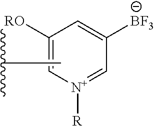

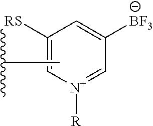

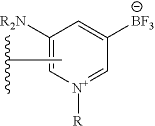

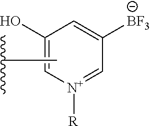

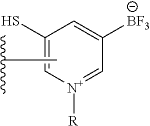

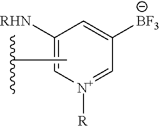

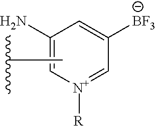

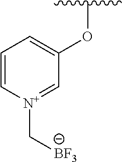

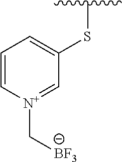

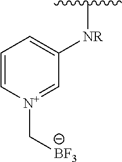

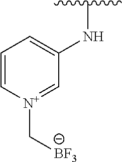

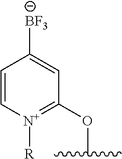

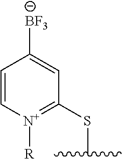

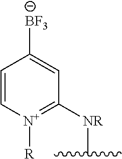

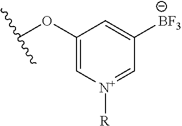

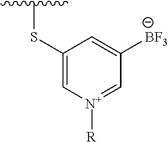

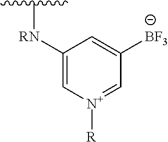

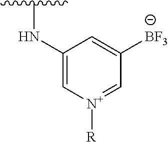

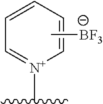

TABLE 3
Exemplary R¹⁷BF₃ groups.

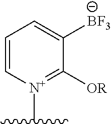

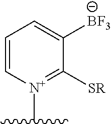

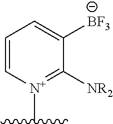

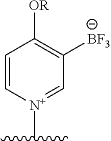

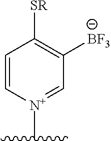

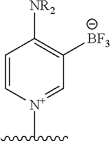

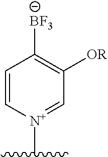

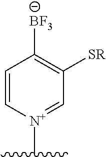

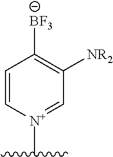

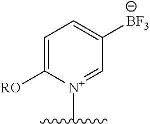

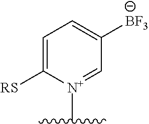

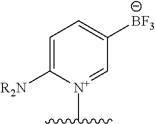

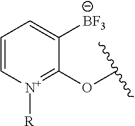

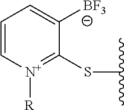

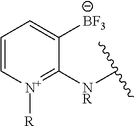

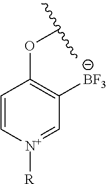

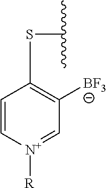

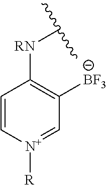

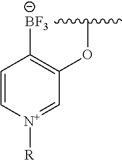

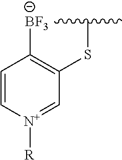

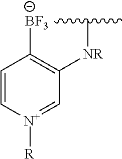

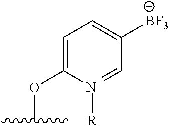

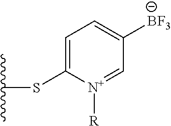

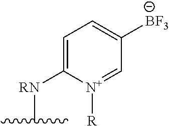

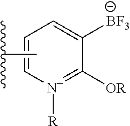

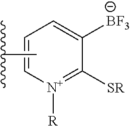

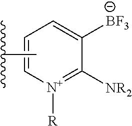

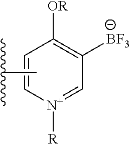

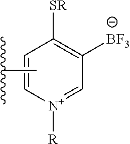

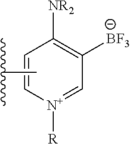

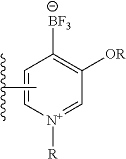

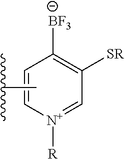

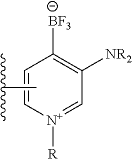

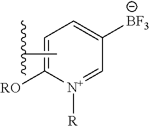

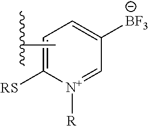

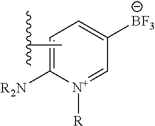

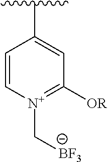

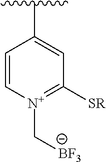

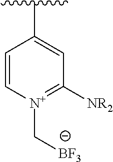

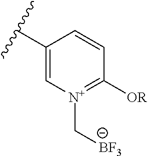

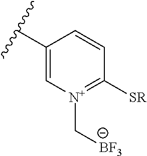

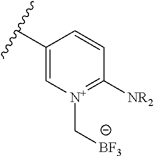

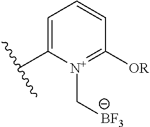

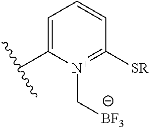

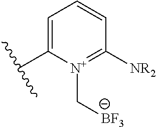

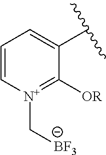

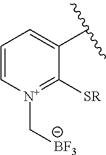

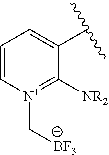

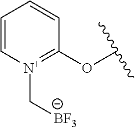

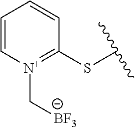

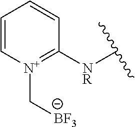

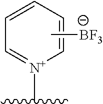

In some embodiments, R¹⁷BF₃ may form

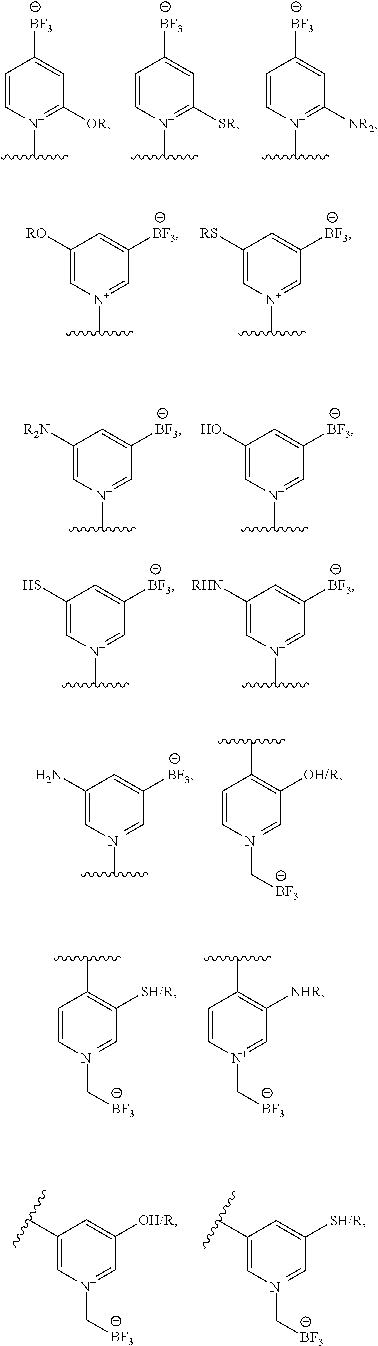

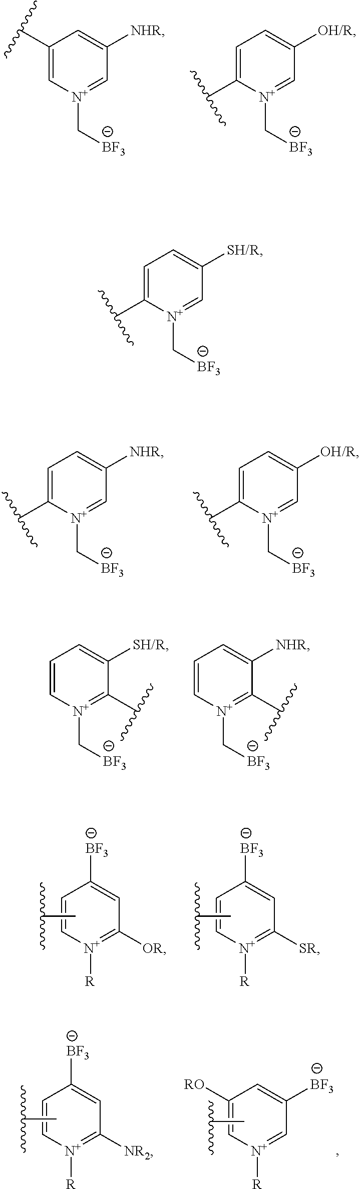

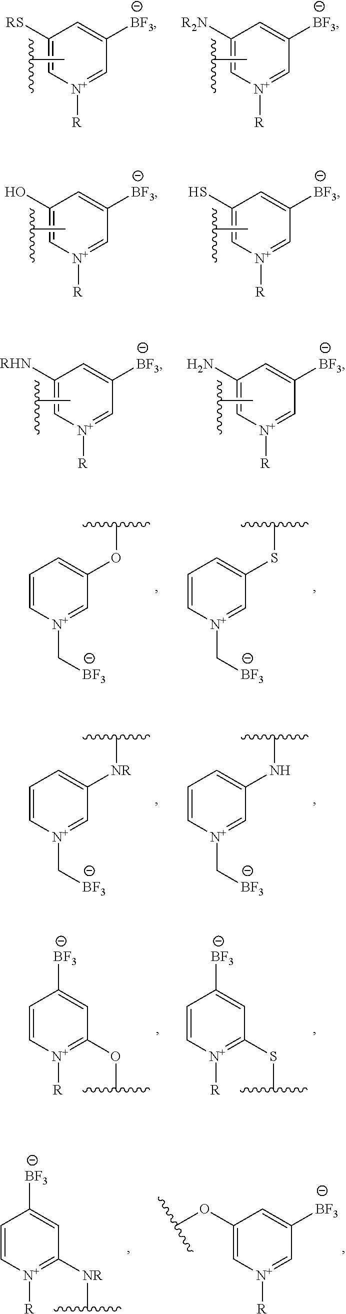

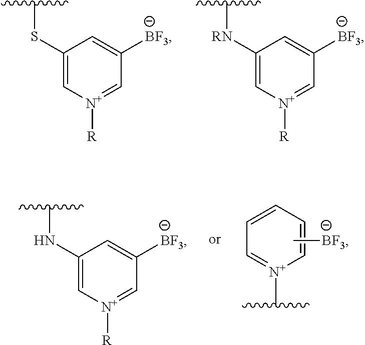

in which the R (when present) in the pyridine substituted —OR, —SR, —NR—, —NHR or —NR₂ is a branched or linear C₁-C₅ alkyl. In some embodiments, R is a branched or linear C₁-C₅ saturated alkyl. In some embodiments, R is methyl. In some embodiments, R is ethyl. In some embodiments, R is propyl. In some embodiments, R is isopropyl. In some embodiments, R is n-butyl. In some embodiments, one fluorine is ¹⁸F. In some embodiments, all three fluorines are ¹⁹F.

In some embodiments, R¹⁷BF₃ may form

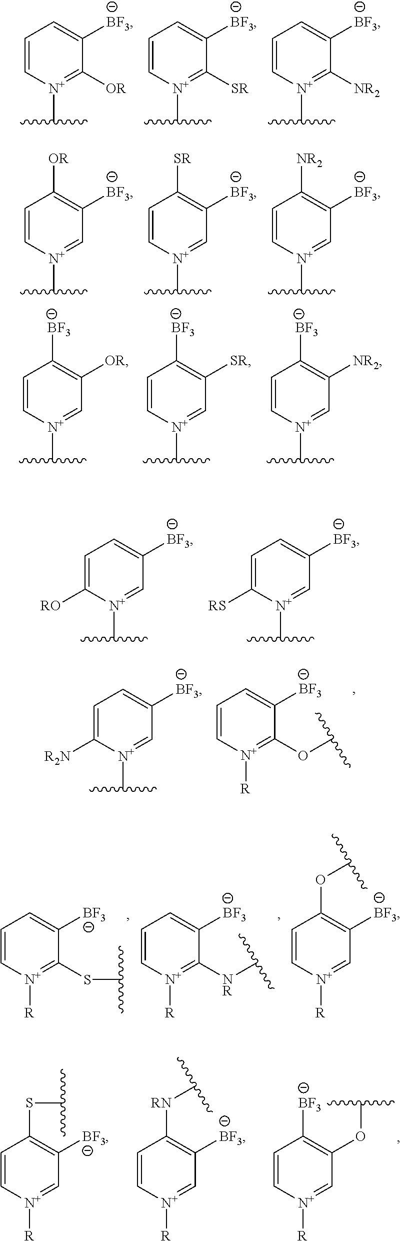

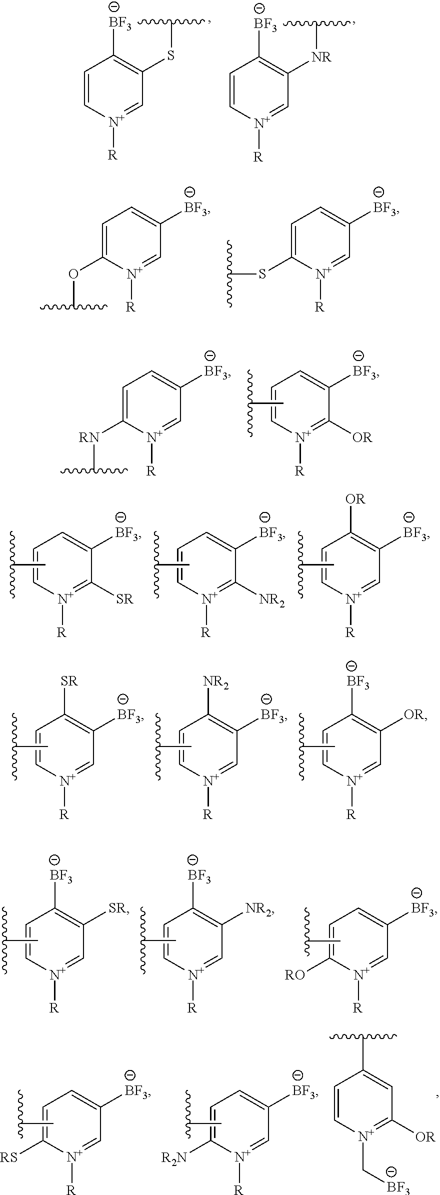

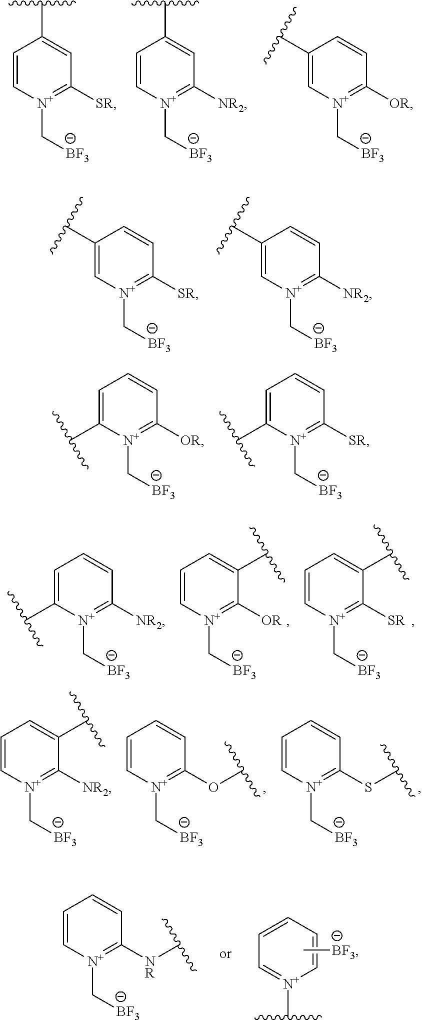

in which the R (when present) in the pyridine substituted —OR, —SR, —NR— or —NR₂ is branched or linear C₁-C₅ alkyl. In some embodiments, R is a branched or linear C₁-C₅ saturated alkyl. In some embodiments, R is methyl. In some embodiments, R is ethyl. In some embodiments, R is propyl. In some embodiments, R is isopropyl. In some embodiments, R is n-butyl. In some embodiments, one or more —R¹⁷BF₃ is

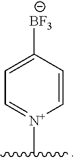

In some embodiments, one fluorine is ¹⁸F. In some embodiments, all three fluorines are ¹⁹F.

In some embodiments, one or more —R¹⁷BF₃ is

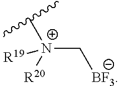

In some embodiments, R¹⁹ is methyl. In some embodiments, R¹⁹ is ethyl. In some embodiments, R¹⁹ is propyl. In some embodiments, R¹⁹ is isopropyl. In some embodiments, R¹⁹ is butyl. In some embodiments, R¹⁹ is n-butyl. In some embodiments, R¹⁹ is pentyl. In some embodiments, R²⁰ is methyl. In some embodiments, R²⁰ is ethyl. In some embodiments, R²⁰ is propyl. In some embodiments, R²⁰ is isopropyl. In some embodiments, R²⁰ is butyl. In some embodiments, R²⁰ is n-butyl. In some embodiments, R²⁰ is pentyl. In some embodiments, R¹⁹ and R²⁰ are both methyl. In some embodiments, one fluorine is ¹⁸F. In some embodiments, all three fluorines are ¹⁹F.

In some embodiments, one or more R^{X} may comprise a prosthetic group containing a silicon-fluorine-acceptor moiety. In some embodiments, the fluorine of the silicon-fluorine acceptor moiety is ¹⁸F. The prosthetic groups containing a silicon-fluorine-acceptor moiety may be independently selected from one or a combination of the following:

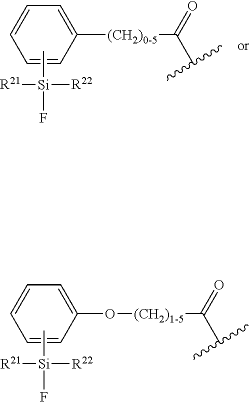

or wherein R²¹ and R²² are independently a linear or branched, cyclic or acyclic, and/or aromatic or non-aromatic C₁-C₁₀ alkyl, alkenyl or alkynyl group. In some embodiments, R²¹ and R²² are independently selected from the group consisting of phenyl, tert-butyl, sec-propyl or methyl. In some embodiments, the prosthetic group is

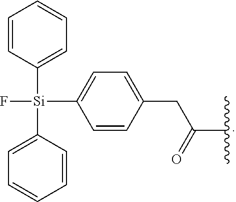 [SMILES: *C(=O)Cc1ccc([Si](F)(c2ccccc2)c2ccccc2)cc1]

In some embodiments, the prosthetic group is

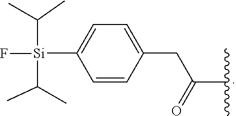 [SMILES: *C(=O)Cc1ccc([Si](F)(C(C)C)C(C)C)cc1]

In some embodiments, the prosthetic group is

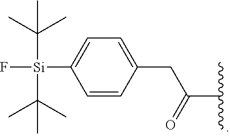 [SMILES: *C(=O)Cc1ccc([Si](F)(C(C)(C)C)C(C)(C)C)cc1]

In some embodiments, the prosthetic group is

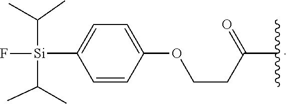 [SMILES: *C(=O)CCOc1ccc([Si](F)(C(C)C)C(C)C)cc1]

In some embodiments, one or more R^{X} comprise a prosthetic group containing a fluorophosphate. In some embodiments, one or more R^{X} comprise a prosthetic group containing a fluorosulfate. In some embodiments, one or more R^{X} comprise a prosthetic group containing a sulfonylfluoride. Such prosthetic groups are well known and are commercially available, and are facile to attach (e.g., via an amide linkage). In some embodiments, the fluorine atom in the fluorophosphate, fluorosulfate or sulfonylfluoride is ¹⁸F. In some embodiments, the fluorine atom in the fluorophosphate, fluorosulfate or sulfonylfluoride is ¹⁹F.

In some embodiments of the compounds of Formula I, II, or III, the compound is selected from the group consisting of:

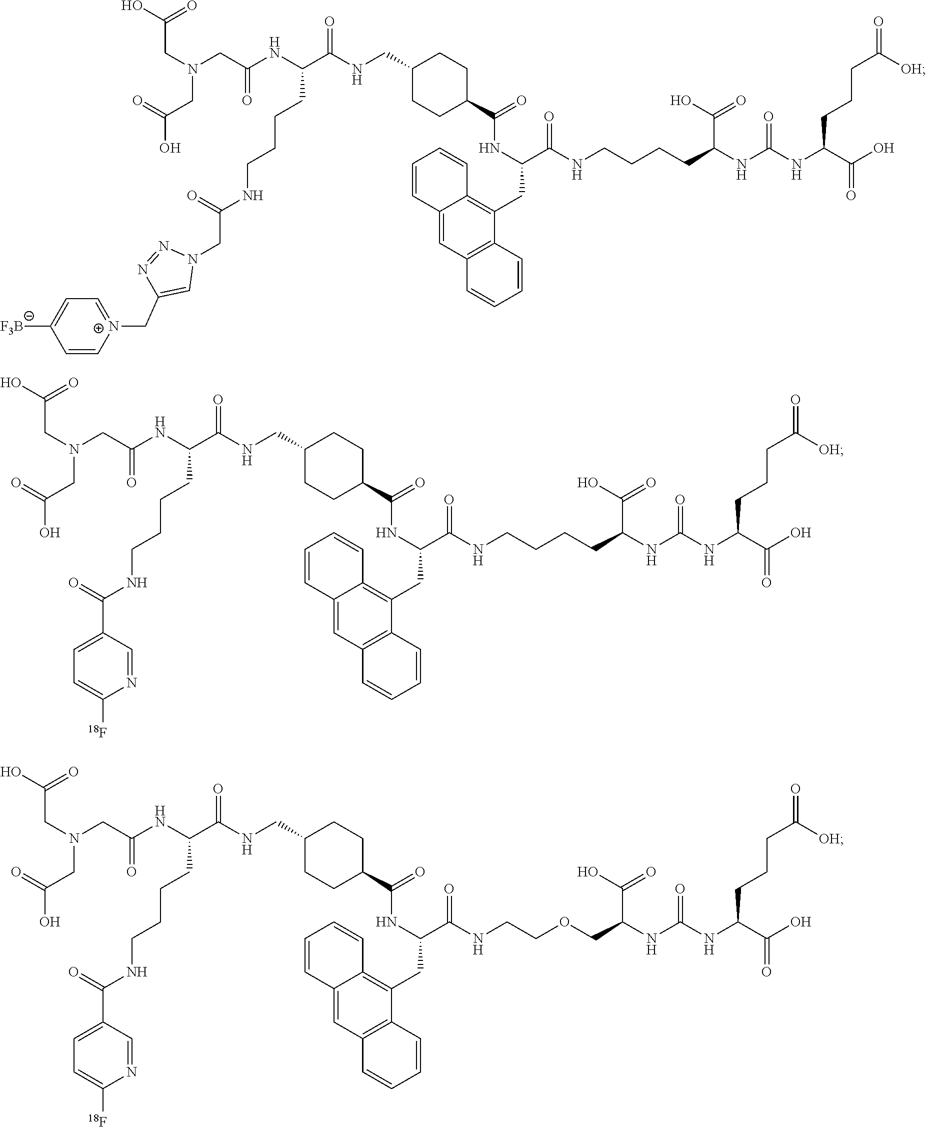

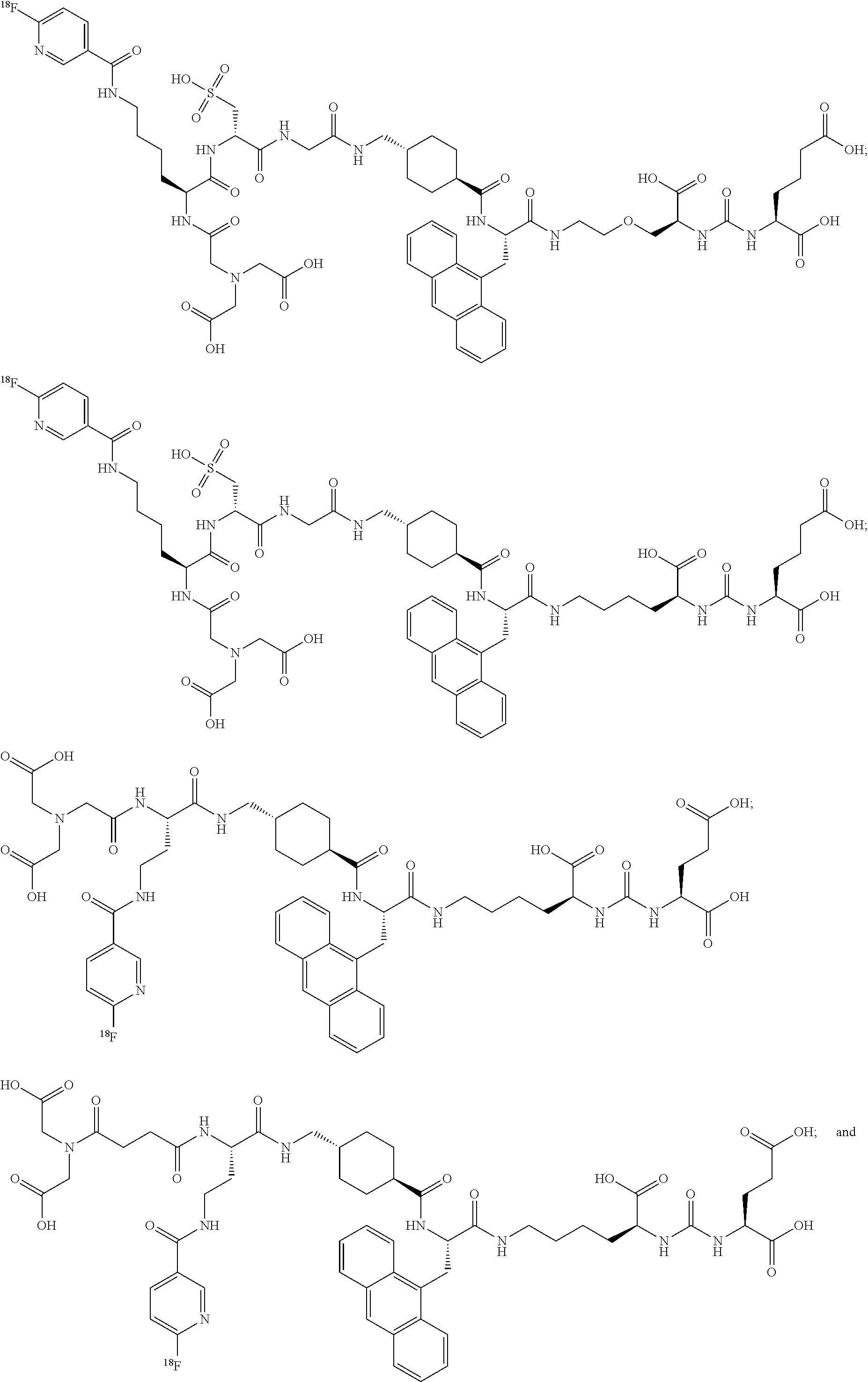

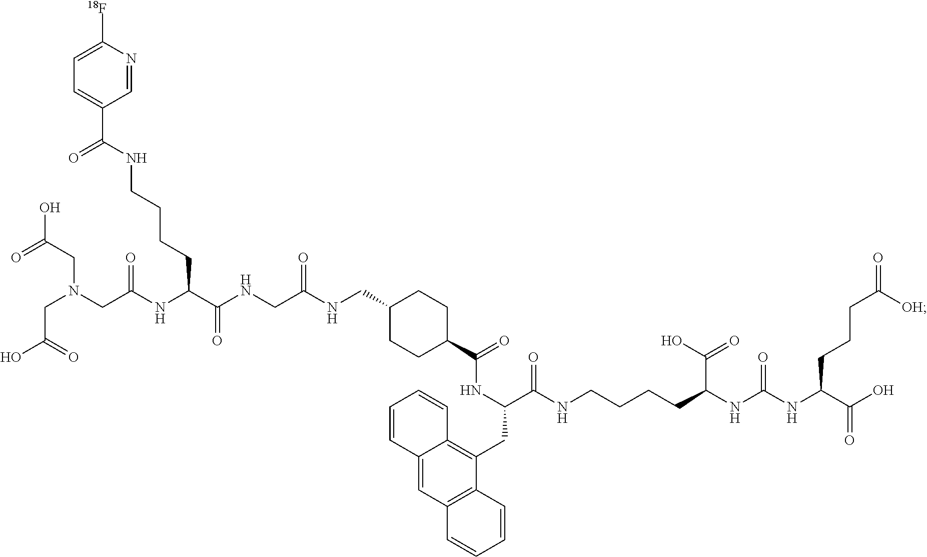 [SMILES: O=C(O)CCC[C@H](NC(=O)N[C@@H](CCCCNC(=O)[C@H](Cc1c2ccccc2cc2ccccc12)NC(=O)[C@H]1CC[C@H](CNC(=O)CNC(=O)[C@H](CCCCNC(=O)c2ccc([18F])nc2)NC(=O)CN(CC(=O)O)CC(=O)O)CC1)C(=O)O)C(=O)O]

or a salt or solvate thereof.

In some embodiments of the compounds of Formula I, II, or III, the compound is:

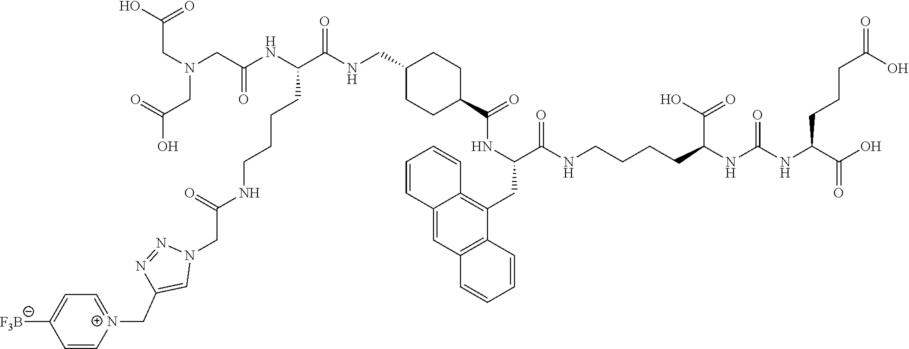

or a salt or solvate thereof; wherein at least one fluorine is ¹⁸F.

The present disclosure also relates to the compounds selected from the group consisting of:

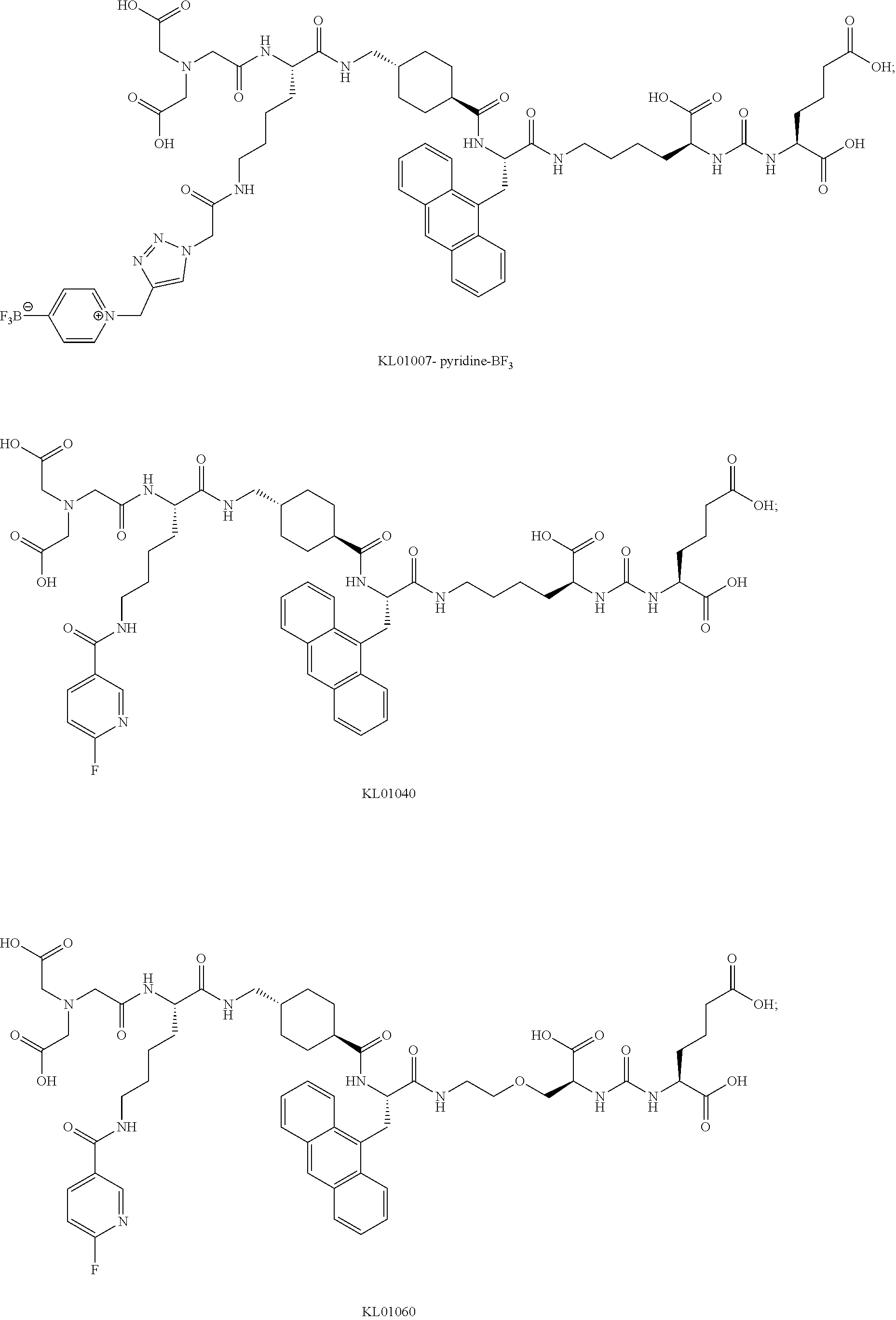

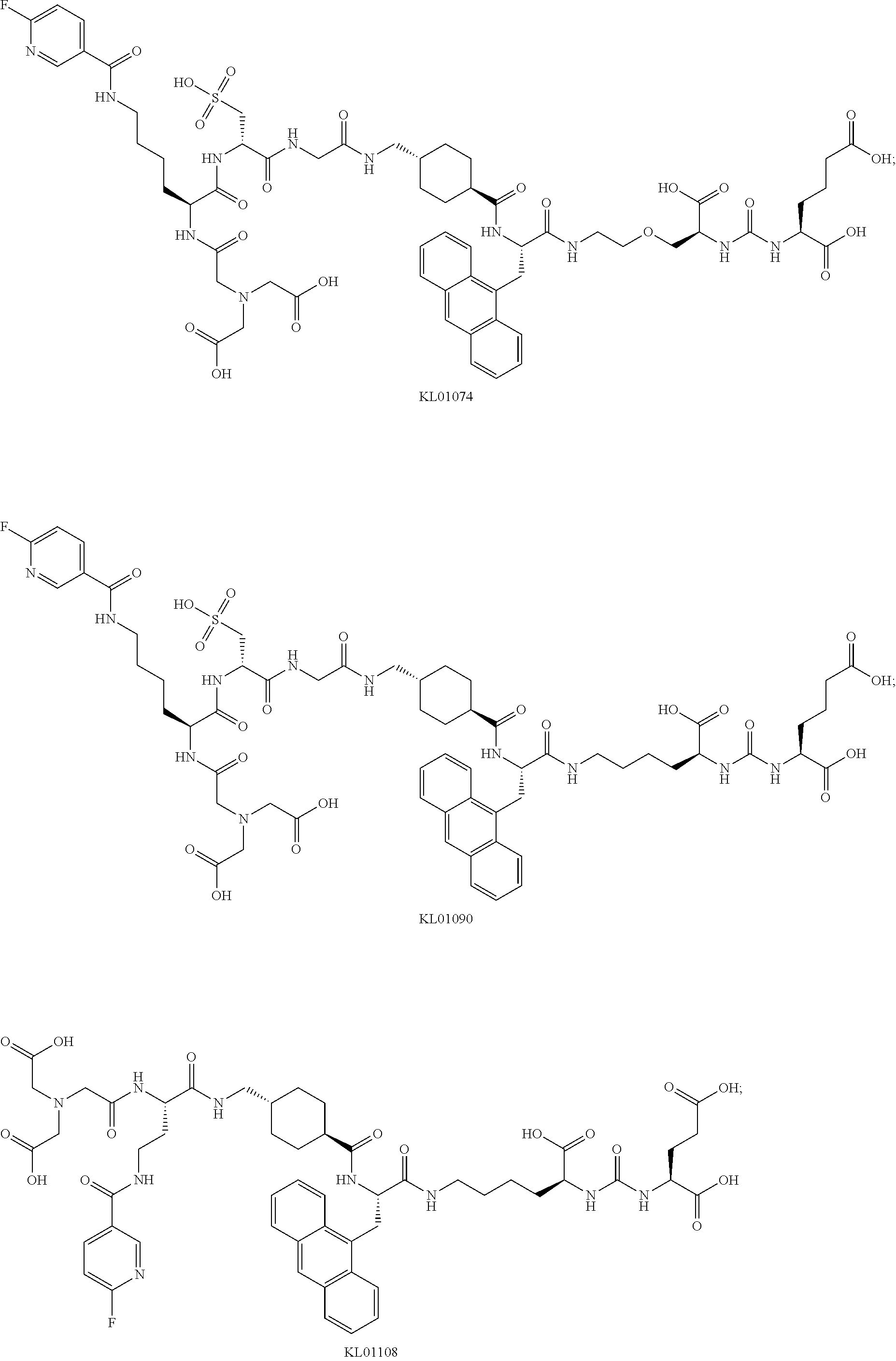

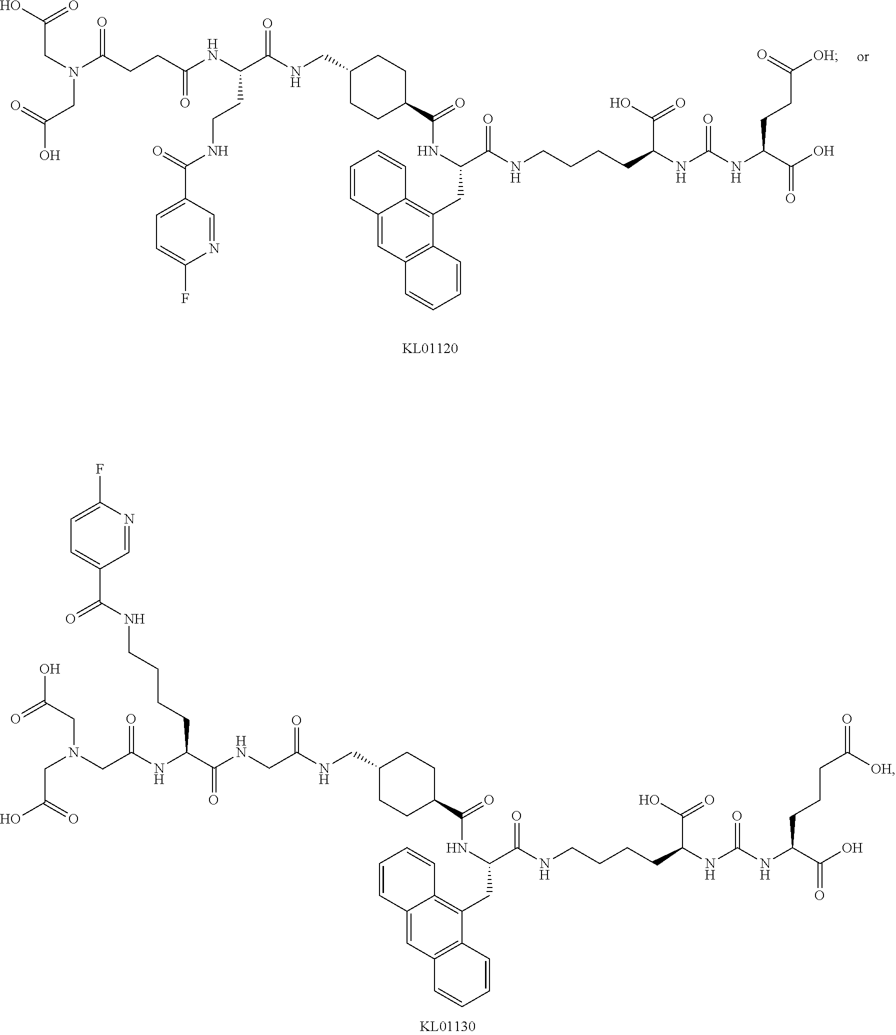 [SMILES: O=C(O)CC[C@H](NC(=O)N[C@@H](CCCCNC(=O)[C@H](Cc1c2ccccc2cc2ccccc12)NC(=O)[C@H]1CC[C@H](CNC(=O)[C@H](CCNC(=O)c2ccc(F)nc2)NC(=O)CCC(=O)N(CC(=O)O)CC(=O)O)CC1)C(=O)O)C(=O)O ; O=C(O)CCC[C@H](NC(=O)N[C@@H](CCCCNC(=O)[C@H](Cc1c2ccccc2cc2ccccc12)NC(=O)[C@H]1CC[C@H](CNC(=O)CNC(=O)[C@H](CCCCNC(=O)c2ccc(F)nc2)NC(=O)CN(CC(=O)O)CC(=O)O)CC1)C(=O)O)C(=O)O]

or a salt or solvate thereof. In some embodiments, the compound is radiolabeled with ¹⁸F.

### METHODS OF USE

When the radiolabeling group comprises or is conjugated to a diagnostic radioisotope, there is disclosed use of certain embodiments of compounds of Formula I, II, or III or salt or solvate thereof for preparation of a radiolabeled tracer for imaging PSMA-expressing tissues in a subject. There is also disclosed a method of imaging PSMA-expressing tissues in a subject, in which the method comprises: administering to the subject a composition comprising certain embodiments of compounds of Formula I, II, or III or salt or solvate thereof and a pharmaceutically acceptable excipient; and imaging tissue of the subject, e.g., using PET or SPECT. When the tissue is a diseased tissue (e.g., a PSMA-expressing cancer), PSMA-targeted treatment may then be selected for treating the subject.

PSMA expression has been detected in various cancers (e.g., Rowe et al., 2015, Annals of Nuclear Medicine 29:877-882; Sathekge et al., 2015, Eur J Nucl Med Mol Imaging 42:1482-1483; Verburg et al., 2015, Eur J Nucl Med Mol Imaging 42:1622-1623; and Pyka et al., J Nucl Med Nov. 19, 2015 jnumed.115.164442). Accordingly, without limitation, the PSMA-expressing cancer may be prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer or sarcoma. In some embodiments, the cancer is prostate cancer.

### Methods of Preparation

The compounds presented herein incorporate peptides, which may be synthesized by any of a variety of methods established in the art. This includes but is not limited to liquid-phase as well as solid-phase peptide synthesis using methods employing 9-fluorenylmethoxycarbonyl (Fmoc) and/or t-butyloxycarbonyl (Boc) chemistries, and/or other synthetic approaches.

Solid-phase peptide synthesis methods and technology are well-established in the art. For example, peptides may be synthesized by sequential incorporation of the amino acid residues of interest one at a time. In such methods, peptide synthesis is typically initiated by attaching the C-terminal amino acid of the peptide of interest to a suitable resin. Prior to this, reactive side chain and alpha amino groups of the amino acids are protected from reaction by suitable protecting groups, allowing only the alpha carboxyl group to react with a functional group such as an amine group, a hydroxyl group, or an alkyl halide group on the solid support. Following coupling of the C-terminal amino acid to the support, the protecting group on the side chain and/or the alpha amino group of the amino acid is selectively removed, allowing the coupling of the next amino acid of interest. This process is repeated until the desired peptide is fully synthesized, at which point the peptide can be cleaved from the support and purified. A non-limiting example of an instrument for solid-phase peptide synthesis is the Aapptec Endeavor 90 peptide synthesizer.

To allow coupling of additional amino acids, Fmoc protecting groups may be removed from the amino acid on the solid support, e.g., under mild basic conditions, such as piperidine (20-50% v/v) in DMF. The amino acid to be added must also have been activated for coupling (e.g., at the alpha carboxylate). Non-limiting examples of activating reagents include without limitation 2-(1H-benzotriazol-1-yl)-1,1,3,3-tetramethyluronium hexafluorophosphate (HBTU), 2-(1H-benzotriazol-1-yl)-1,1,3,3-tetramethyluronium tetrafluoroborate (TBTU), 2-(7-Aza-1H-benzotriazole-1-yl)-1,1,3,3-tetramethyluronium hexafluorophosphate (HATU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP). Racemization is minimized by using triazoles, such as 1-hydroxy-benzotriazole (HOBt) and 1-hydroxy-7-aza-benzotriazole (HOAt). Coupling may be performed in the presence of a suitable base, such as N,N-diisopropylethylamine (DIPEA/DIEA) and the like. For long peptides or if desired, peptide synthesis and ligation may be used.

Apart from forming typical peptide bonds to elongate a peptide, peptides may be elongated in a branched fashion by attaching to side chain functional groups (e.g., carboxylic acid groups or amino groups), either: side chain to side chain; or side chain to backbone amino or carboxylate. Coupling to amino acid side chains may be performed by any known method, and may be performed on-resin or off-resin. Non-limiting examples include: forming an amide between an amino acid side chain containing a carboxyl group (e.g. Asp, D-Asp, Glu, D-Glu, and the like) and an amino acid side chain containing an amino group (e.g. Lys, D-Lys, Orn, D-Orn, Dab, D-Dab, Dap, D-Dap, and the like) or the peptide N-terminus; forming an amide between an amino acid side chain containing an amino group (e.g. Lys, D-Lys, Orn, D-Orn, Dab, D-Dab, Dap, D-Dap, and the like) and either an amino acid side chain containing a carboxyl group (e.g. Asp, D-Asp, Glu, D-Glu, and the like) or the peptide C-terminus; and forming a 1, 2, 3-triazole via click chemistry between an amino acid side chain containing an azide group (e.g. Lys(N₃), D-Lys(N₃), and the like) and an alkyne group (e.g. Pra, D-Pra, and the like). The protecting groups on the appropriate functional groups must be selectively removed before amide bond formation, whereas the reaction between an alkyne and an azido groups via the click reaction to form an 1,2,3-triazole does not require selective deprotection. Non-limiting examples of selectively removable protecting groups include 2-phenylisopropyl esters (O-2-PhiPr) (e.g. on Asp/Glu) as well as 4-methyltrityl (Mtt), allyloxycarbonyl (alloc), 1-(4,4-dimethyl-2,6-dioxocyclohex-1-ylidene))ethyl (Dde), and 1-(4,4-dimethyl-2,6-dioxocyclohex-1-ylidene)-3-methylbutyl (ivDde) (e.g. on Lys/Orn/Dab/Dap). O-2-PhiPr and Mtt protecting groups can be selectively deprotected under mild acidic conditions, such as 2.5% trifluoroacetic acid (TFA) in DCM. Alloc protecting groups can be selectively deprotected using tetrakis(triphenylphosphine)palladium(0) and phenyl silane in DCM. Dde and ivDde protecting groups can be selectively deprotected using 2-5% of hydrazine in DMF. Deprotected side chains of Asp/Glu (L- or D-forms) and Lys/Orn/Dab/Dap (L- or D-forms) can then be coupled, e.g., by using the coupling reaction conditions described above.

Peptide backbone amides may be N-methylated (i.e., alpha amino methylated). This may be achieved by directly using Fmoc-N-methylated amino acids during peptide synthesis. Alternatively, N-methylation under Mitsunobu conditions may be performed. First, a free primary amine group is protected using a solution of 4-nitrobenzenesulfonyl chloride (Ns-Cl) and 2,4,6-trimethylpyridine (collidine) in NMP. N-methylation may then be achieved in the presence of triphenylphosphine, diisopropyl azodicarboxylate (DIAD) and methanol. Subsequently, N-deprotection may be performed using mercaptoethanol and 1,8-diazabicyclo[5.4.0]undec-7-ene (DBU) in NMP. For coupling protected amino acids to N-methylated alpha amino groups, HATU, HOAt and DIEA may be used.

The PSMA-binding moiety (e.g., Lys-ureido-Aad, and the like) may be constructed on solid phase via the formation of a ureido linkage between the amino groups of two amino acids. This can be done by attaching an Fmoc-protecting amino acid (for example Fmoc-Lys(ivDde)-OH) to Wang resin using standard activation/coupling strategy (for example, Fmoc-protected amino acid (4 eq.), HATU (4 eq.) and N,N-diisopropylethylamine (7 eq.) in N,N-dimethylformamide). The Fmoc-protecting group is then removed by 20% piperidine in N,N-dimethylformamide. To form the ureido linkage, the freed amino group of the solid-phase-attached amino acid is reacted with the 2^{nd} amino acid which has its carboxylate group protected with a t-butyl group and its amino group activated and converted to an isocyanate group (—N═C═O). The activation and conversion of an amino group to an isocyanate group can be achieved by reacting the amino group with phosgene or triphosgene. After the formation of the ureido linkage, the side chain functional group of the amino acid (for example ivDde on Lys) can be removed, and then the linker, albumin-binding motif, and/or radiolabeling group (e.g., radiometal chelator and the like) can be subsequently coupled to the PSMA-binding moiety.

The formation of the thioether (—S—) and ether (—O—) linkages (e.g., for R⁴) can be achieved either on solid phase or in solution phase. For example, the formation of thioether (—S—) linkage can be achieved by coupling between a thiol-containing compound (such as the thiol group on cysteine side chain) and an alkyl halide (such as 3-(Fmoc-amino)propyl bromide and the like) in an appropriate solvent (such as N,N-dimethylformamide and the like) in the presence of base (such as N,N-diisopropylethylamine and the like). The formation of an ether (—O—) linkage can be achieved via the Mitsunobu reaction between an alcohol (such as the hydroxyl group on the side chain of serine or threonine, for example) and a phenol group (such as the side chain of tyrosine, for example) in the presence of triphenylphosphine and diisopropyl azidicarboxylate (DIAD) in an aprotic solvent (such as 1,4-dioxane and the like). If the reactions are carried out in solution phase, the reactants used are preferably in equivalent molar ratio (1 to 1), and the desired products can be purified by flash column chromatography or high-performance liquid chromatography (HPLC). If the reactions are carried out on solid phase, meaning one reactant has been attached to a solid phase, then the other reactant is normally used in excess amount (3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

Non-peptide moieties (e.g., radiolabeling groups, albumin-binding groups and/or linkers) may be coupled to the peptide N-terminus while the peptide is attached to the solid support. This is facile when the non-peptide moiety comprises an activated carboxylate (and protected groups if necessary) so that coupling can be performed on resin. For example, but without limitation, a bifunctional chelator, such as 1,4,7,10-tetraazacyclododecane-1,4,7,10-tetraacetic acid (DOTA) tris(tert-butyl ester) may be activated in the presence of N-hydroxysuccinimide (NHS) and N,N′-dicyclohexylcarbodiimide (DCC) for coupling to a peptide. Alternatively, a non-peptide moiety may be incorporated into the compound via a copper-catalyzed click reaction under either liquid or solid phase conditions. Copper-catalyzed click reactions are well established in the art. For example, 2-azidoacetic acid is first activated by NHS and DCC and coupled to a peptide. Then, an alkyne-containing non-peptide moiety may be clicked to the azide-containing peptide in the presence of Cuz'⁰ and sodium ascorbate in water and organic solvent, such as acetonitrile (ACN) and DMF and the like.

The synthesis of radiometal chelators is well-known and many chelators are commercially available (e.g., from Sigma-Aldrich™/Milipore Sigma™ and others). Protocols for conjugation of radiometals to the chelators are also well known (e.g., see Example 1, below). The synthesis of the silicon-fluorine-acceptor moieties can be achieved following previously reported procedures (e.g., Bernard-Gauthier et al. Biomed Res Int. 2014 2014:454503; Kostikov et al. Nature Protocols 2012 7:1956-1963; Kostikov et al. Bioconjug Chem. 2012 18:23:106-114; each of which is incorporated by reference in its entirety). The synthesis or acquisition of radioisotope-substituted aryl groups is likewise facile.

The synthesis of the R¹⁶R¹⁷BF₃ component on the PSMA-targeting compounds can be achieved following previously reported procedures (Liu et al. Angew Chem Int Ed 2014 53:11876-11880; Liu et al. J Nucl Med 2015 55:1499-1505; Liu et al. Nat Protoc 2015 10:1423-1432; Kuo et al. J Nucl Med, in press, doi:10.2967/jnumed.118.216598; each of which is incorporated by reference in its entirety). Generally, the BF₃-containing motif can be coupled to the linker via click chemistry by forming a 1,2,3-triazole ring between a BF₃-containing azido (or alkynyl) group and an alkynyl (or azido) group on the linker, or by forming an amide linkage between a BF₃-containing carboxylate and an amino group on the linker. To make the BF₃-containing azide, alkyne or carboxylate, a boronic acid ester-containing azide, alkyne or carboxylate is first prepared following by the conversion of the boronic acid ester to BF₃ in a mixture of HCl, DMF and KHF₂. For alkyl BF₃, the boronic acid ester-containing azide, alkyne or carboxylate can be prepared by coupling boronic acid ester-containing alkyl halide (such as iodomethylboronic acid pinacol ester) with an amine-containing azide, alkyne or carboxylate (such as N, N-dimethylpropargylamine). For aryl BF₃, the boronic acid ester can be prepared via Suzuki coupling using aryl halide (iodine or bromide) and bis(pinacolato)diboron. [00261]¹⁸F-Fluorination of the BF₃-containing PSMA-targeting compounds via ¹⁸F-¹⁹F isotope exchange reaction can be achieved following previously published procedures (Liu et al. Nat Protoc 2015 10:1423-1432, incorporated by reference in its entirety). Generally, ~100 nmol of the BF₃-containing compound is dissolved in a mixture of 15 μl of pyridazine-HCl buffer (pH=2.0-2.5, 1 M), 15 μl of DMF and 1 μl of a 7.5 mM KHF₂ aqueous solution. ¹⁸F-Fluoride solution (in saline, 60 μl) is added to the reaction mixture, and the resulting solution is heated at 80° C. for 20 min. At the end of the reaction, the desired product can be purified by solid phase extraction or by reversed high performance liquid chromatography (HPLC) using a mixture of water and acetonitrile as the mobile phase.

When the peptide has been fully synthesized on the solid support, the desired peptide may be cleaved from the solid support using suitable reagents, such as TFA, tri-isopropylsilane (TIS) and water. Side chain protecting groups, such as Boc, pentamethyldihydrobenzofuran-5-sulfonyl (Pbf), trityl (Trt) and tert-butyl (tBu) are simultaneously removed (i.e., deprotection). The crude peptide may be precipitated and collected from the solution by adding cold ether followed by centrifugation. Purification and characterization of the peptides may be performed by standard separation techniques, such as high performance liquid chromatography (HPLC) based on the size, charge and polarity of the peptides. The identity of the purified peptides may be confirmed by mass spectrometry or other similar approaches.

### EXAMPLES

The present invention will be further illustrated in the following examples.

### Example 1: Synthesis of KL01007-Pyridine-BF₃, KL01040, KL01060, KL01074, KL01090, KL01108, KL01120, and KL01130

### General Methods

PSMA-targeting ligands were synthesized on solid phase synthesis via either AAPPTec (Louisville, KY) Endeavor 90 Peptide Synthesizer or CEM Corporation (Matthews, NC) Liberty Blue Automated Microwave Peptide Synthesizer starting with commercially obtained resins via standard Fmoc chemistry. Final cleavage and deprotection were achieved with 95% trifluoroacetic acid (TFA) and 5% triisopropylsilane (TIS). Purification and quality control of standards, precursors, and ¹⁸F-labeled tracers were performed on Agilent (Santa Clara, CA) HPLC Systems equipped with a model 1200 quaternary pump, a model 1200 UV absorbance detector (set at 220 nm), and a Bioscan (Washington, DC) Nal scintillation detector. The operation of Agilent HPLC systems was controlled using the Agilent ChemStation software. The HPLC columns used were a semi-preparative column (Luna C18, 5 μm particle size, 100 Å pore size, 250×10 mm) and an analytical column (Luna C18, 5 μm particle size, 100 Å pore size, 250×4.6 mm) from Phenomenex (Torrance, CA) to collected desired peaks and to verify purity, respectively. Collected peaks with the desired product were then lyophilized using Labconco (Kansas City, MO) FreeZone 4.5 Plus freeze drier. Identity of PSMA-targeting ligands was verified with mass via AB SCIEX (Framingham, MA) 4000 QTRAP mass spectrometer system with an ESI ion source. C18 Sep-Pak cartridges (1 cm³, 50 mg) were obtained from Waters (Milford, MA). The radioactivity of collected mice tissues from biodistribution studies was counted using a PerkinElmer (Waltham, MA) Wizard2 2480 automatic gamma counter.

### Synthesis of KL01007-Pyridine-BF₃

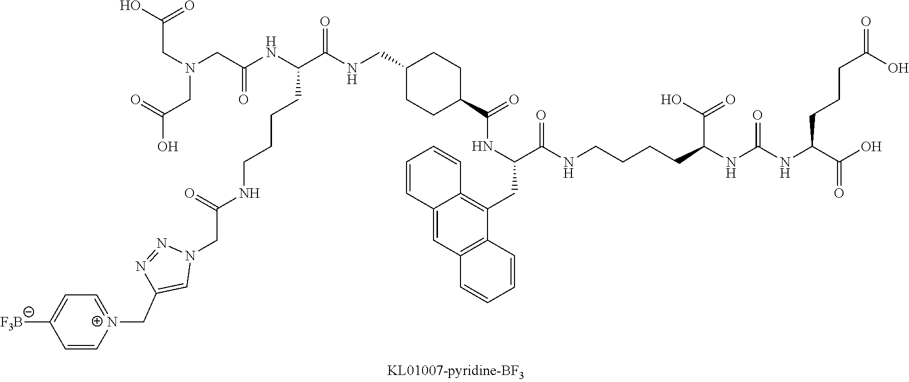

The PSMA-targeting pharmacophore Lys-urea-Aad (Aad: 2-aminoadipic acid) was synthesized via solid-phase peptide chemistry starting with 1 equivalent of Fmoc-Lys(ivDde)-Wang resin. After swelling in N,N-dimethylformamide (DMF), Fmoc was removed from resin via the treatment of 20% piperidine in DMF. The isocyanate of 2-aminoadipyl component was generated by combining 3-4 equivalents L-2-aminoadipic acid di-tertbutyl ester in dichloromethane and diisopropylethylamine (DIPEA) in dry ice/acetone bath. Triphosgene (3 equivalents) dissolved in dichloromethane (DCM) was added dropwise to the reaction and then warmed to room temperature and stirred for 30 min to generate the isocyanate of 2-aminoadipyl component for addition to resin. The activated Aad was then added to the resin and reacted for 24 hr to form urea linkage. After washing the resin, the ivDde-protection group was removed with 5% hydrazine in DMF. Fmoc-L-9-anthrylalanine (5 equivalents) was coupled to Lys with 5 equivalents of (1-[bis(dimethylamino)methylene]-1H-1,2,3-triazolo[4,5-b]pyridinium 3-oxide hexafluorophosphate (HATU) and 10 equivalents of DIPEA under microwave for 10 min at 75° C. Following Fmoc deprotection, the following sequences were added: Fmoc-tranexamic acid and followed by Fmoc-Lys(ivDde). Then the Fmoc from the Lys residue is first deprotected to add N,N-bis[2-(tert-butoxy)-2-oxoethyl]glycine and then ivDde deprotection to add an activated azidoacetic acid. The peptides were then cleaved and deprotected by 95% TFA and 5% TIS for 2 hr at room temperature. Resin was filtered and the peptide precipitated via addition to cold diethyl ether. The crude was initially purified before doing click reaction to attach pyridine-BF₃. Azide-peptide (1 equivalent) was reacted overnight with 5 equivalents of pyridine-BF₃ and CuSO₄, 10 equivalent of sodium ascorbate and 100 μL of 1.0 M K₂CO₃ for buffer. Final product was purified by HPLC using the semi-preparative column. HPLC conditions: 4.5 mL/min flow rate, 38% acetonitrile (MeCN) and 0.1% formic acid in water, retention time was 7 min. The yield of final product was 3.7%. Calculated ESI-MS for KL01007-pyridine-BF₃ (C₆₀H₇₆BF₃N₁₂O₁₆) [M+1H-1F]²⁺: 635.3; found 635.4.

### Synthesis of KL01040 and its Radiolabeling Precursor

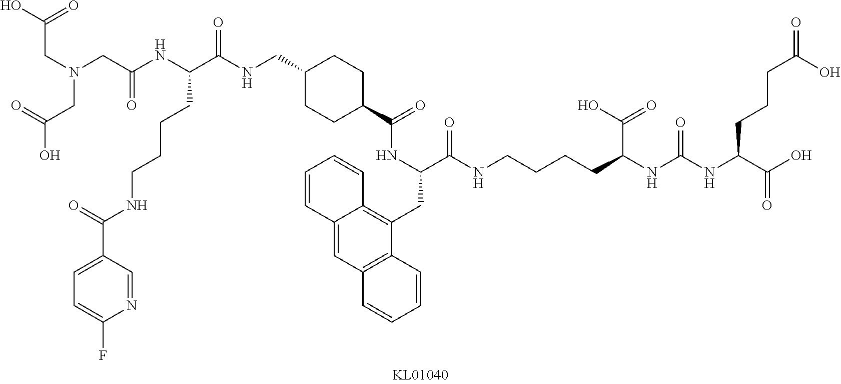 [SMILES: O=C(O)CCC[C@H](NC(=O)N[C@@H](CCCCNC(=O)[C@H](Cc1c2ccccc2cc2ccccc12)NC(=O)[C@H]1CC[C@H](CNC(=O)[C@H](CCCCNC(=O)c2ccc(F)nc2)NC(=O)CN(CC(=O)O)CC(=O)O)CC1)C(=O)O)C(=O)O]

Synthesis of KL01040 and its radiolabeling precursor follow the same procedures for the synthesis of KL01007-pyridine-BF₃ up to N,N-bis[2-(tert-butoxy)-2-oxoethyl]glycine coupling and ivDde deprotection as described above. Following the last ivDde deprotection. The radiolabeling precursor and non-radioactive standard of KL01040 were then generated by coupling N,N,N-trimethyl-5-((2,3,5,6-tetrafluorophenoxy)-carbonyl-pyridine-2-aminiumchloride and 6-fluoronicotinic acid respectively. Non-radioactive KL01040 standard prepared by adding 6-fluoronicotinic acid was completed via microwave method at 75° C. for 10 min. The radiolabeling precursor was formed via room temperature coupling by dissolving 3 equivalents of N,N,N-trimethyl-5-((2,3,5,6-tetrafluorophenoxy)-carbonyl-pyridine-2-aminiumchloride in 2 mL of DMF and 3 equivalents of triethylamine (TEA) and then conducting coupling for 2 hr. Both compounds were purified with HPLC semi-preparative column: 42% MeCN and 0.1% formic acid in water with retention around 9 min for the non-radioactive KL01040 standard; 29% MeCN and 0.1% formic acid in water with retention around 8.6 min for the radiolabeling precursor. The final yields were 32% and 35% for the non-radioactive KL01040 standard and radiolabeling precursor, respectively. The calculated ESI-MS for the non-radioactive KL01040 standard [M+H]⁺ was 1144.5; found 1144.8. The calculated ESI-MS for the radiolabeling precursor [M+H]⁺ was 1184.6; found 1184.9.

### Synthesis of KL01060 and its Radiolabeling Precursor

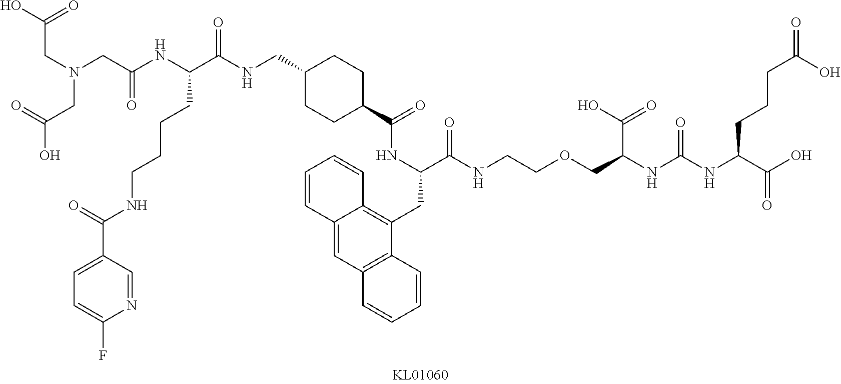 [SMILES: O=C(O)CCC[C@H](NC(=O)N[C@@H](COCCNC(=O)[C@H](Cc1c2ccccc2cc2ccccc12)NC(=O)[C@H]1CC[C@H](CNC(=O)[C@H](CCCCNC(=O)c2ccc(F)nc2)NC(=O)CN(CC(=O)O)CC(=O)O)CC1)C(=O)O)C(=O)O]

Three equivalents of Fmoc-Oxalysine(Alloc) were loaded to 2-chlorotrityl chloride resin with 4 equivalents of DIPEA in DCM for overnight reaction. Formation of urea bond to Aad was synthesized via solid-phase peptide chemistry starting with 1 equivalent of Fmoc-Oxalysine(Alloc)-2-chlorotrityl resin. After swelling in N,N-dimethylformamide (DMF), Fmoc was removed from resin via the treatment of 20% piperidine in DMF. The isocyanate of 2-aminoadipyl component was generated by combining 3-4 equivalents L-2-aminoadipic acid di-tertbutyl ester in dichloromethane and diisopropylethylamine (DIPEA) in dry ice/acetone bath. Triphosgene (3 equivalents) dissolved in dichloromethane (DCM) was added dropwise to the reaction and then warmed to room temperature and stirred for 30 min to generate the isocyanate of 2-aminoadipyl component for addition to resin. The activated Aad was then added to the resin and reacted for 24 hr to form urea linkage. After washing the resin, the Alloc-protection group was removed with 0.25 equivalent of tetrakis(triphenylphosphine)palladium (0) and 20 equivalents of phenylsilane in DCM 2 times at room temperature. Fmoc-L-9-anthrylalanine (5 equivalents) was coupled to resin with 5 equivalents of (1-[bis(dimethylamino)methylene]-1H-1,2,3-triazolo[4,5-b]pyridinium 3-oxide hexafluorophosphate (HATU) and 10 equivalents of DIPEA under microwave for 10 min at 75° C. Following Fmoc deprotection, the following sequences were added: Fmoc-tranexamic acid and followed by Fmoc-Lys(ivDde). Then the Fmoc from the Lys residue is first deprotected to add N,N-bis[2-(tert-butoxy)-2-oxoethyl]glycine. ivDde is then deprotected and the radiolabeling precursor and non-radioactive standard of KL01060 were then generated by coupling N,N,N-trimethyl-5-((2,3,5,6-tetrafluorophenoxy)-carbonyl-pyridine-2-aminiumchloride and 6-fluoronicotinic acid, respectively, according to previous methods. The precursor was purified with HPLC semi-preparative column: 28% MeCN and 0.1% formic acid in water with retention around 10.6 min. The final yield was 13% for the radiolabeling precursor. The calculated ESI-MS for the radiolabeling precursor [M+H]⁺ was 1186.6; found 1186.1.

### Synthesis of KL01074 and its Radiolabeling Precursor

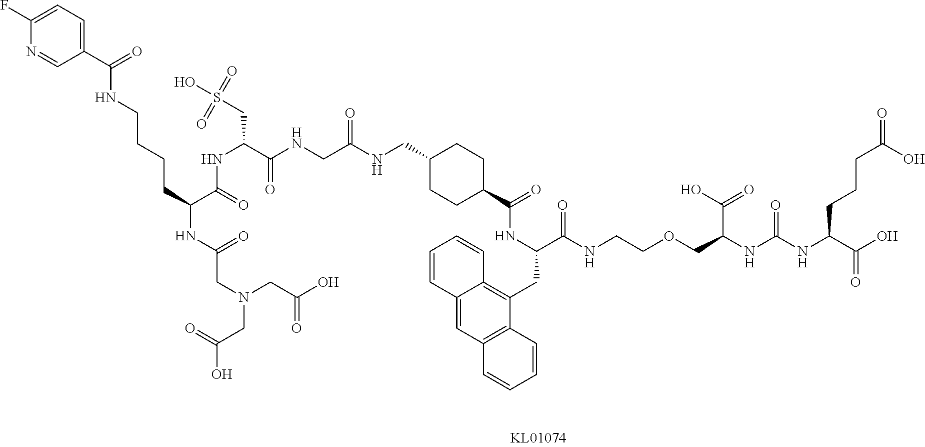 [SMILES: O=C(O)CCC[C@H](NC(=O)N[C@@H](COCCNC(=O)[C@H](Cc1c2ccccc2cc2ccccc12)NC(=O)[C@H]1CC[C@H](CNC(=O)CNC(=O)[C@@H](CS(=O)(=O)O)NC(=O)[C@H](CCCCNC(=O)c2ccc(F)nc2)NC(=O)CN(CC(=O)O)CC(=O)O)CC1)C(=O)O)C(=O)O]

Starting with Fmoc-Oxalysine(Alloc)-2-chlorotrityl resin as for the synthesis of KL01060, urea bond was formed to Aad for solid phase coupling. The resin then underwent Alloc deprotection, followed by addition of the following sequence Fmoc-L-9-anthrylalanine (5 equivalents), Fmoc-tranexamic acid, Fmoc-Gly, Fmoc-L-cysteic acid, and Fmoc-Lys(ivDde). Then the Fmoc from the Lys residue is first deprotected to add N,N-bis[2-(tert-butoxy)-2-oxoethyl]glycine. ivDde is then deprotected and the precursor and non-radioactive standard of KL01074 were then generated via coupling N,N,N-trimethyl-5-((2,3,5,6-tetrafluorophenoxy)-carbonyl-pyridine-2-aminiumchloride and 6-fluoronicotinic acid, respectively, according to previous methods. Standard was purified with HPLC semi-preparative column: 37% MeCN and 0.1% formic acid in water with retention around 7.9 min for the radiolabeling precursor. The final yield was 8% for the nonradioactive KL01074 radiolabeling precursor. The calculated ESI-MS for the nonradioactive KL01074 standard [M+H]⁺ was 677.8. The calculated ESI-MS for the radiolabeling precursor [M+H]⁺ was 697.8; found 697.9.

### Synthesis of KL01090 and its Radiolabeling Precursor

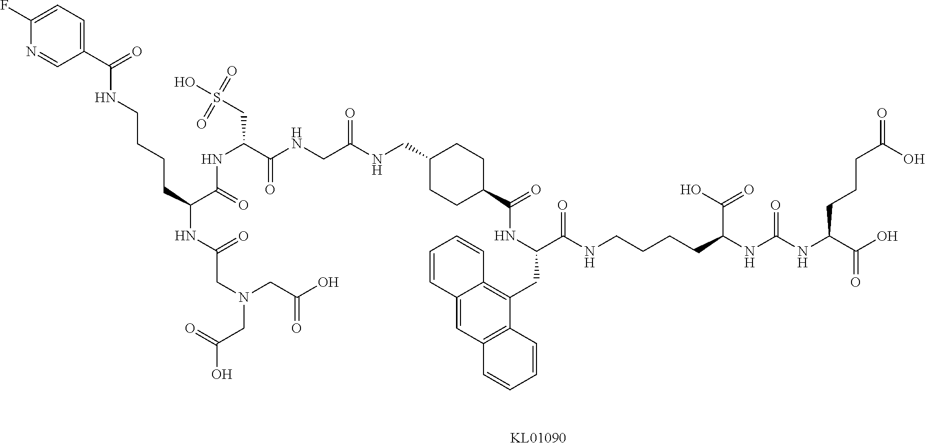 [SMILES: O=C(O)CCC[C@H](NC(=O)N[C@@H](CCCCNC(=O)[C@H](Cc1c2ccccc2cc2ccccc12)NC(=O)[C@H]1CC[C@H](CNC(=O)CNC(=O)[C@@H](CS(=O)(=O)O)NC(=O)[C@H](CCCCNC(=O)c2ccc(F)nc2)NC(=O)CN(CC(=O)O)CC(=O)O)CC1)C(=O)O)C(=O)O]

Starting with Fmoc-Lys(ivDde)-Wang resin, urea bond was formed to Aad for solid phase coupling. The resin then underwent ivDde deprotection, followed by addition of the following sequence Fmoc-L-9-anthrylalanine (5 equivalents), Fmoc-tranexamic acid, Fmoc-Gly, Fmoc-L-cysteic acid, and Fmoc-Lys(ivDde). Then the Fmoc from the Lys residue is first deprotected to add N,N-bis[2-(tert-butoxy)-2-oxoethyl]glycine. ivDde is then deprotected and the radiolabeling precursor and non-radioactive standard of KL01090 were then generated via coupling N,N,N-trimethyl-5-((2,3,5,6-tetrafluorophenoxy)-carbonyl-pyridine-2-aminiumchloride and 6-fluoronicotinic acid, respectively, according to previous methods. Precursor was purified with QC column: 27% MeCN and 0.1% formic acid in water with retention around 8 min for the radiolabeling precursor. The final yield was 30% for the radiolabeling precursor. The calculated ESI-MS for the nonradioactive KL01090 standard [M+2H]²⁺ was 676.8; found 675.4. The calculated ESI-MS for the radiolabeling precursor [M+2H]²⁺ was 696.8; found 696.2.

### Synthesis of KL01108 and its Radiolabeling Precursor

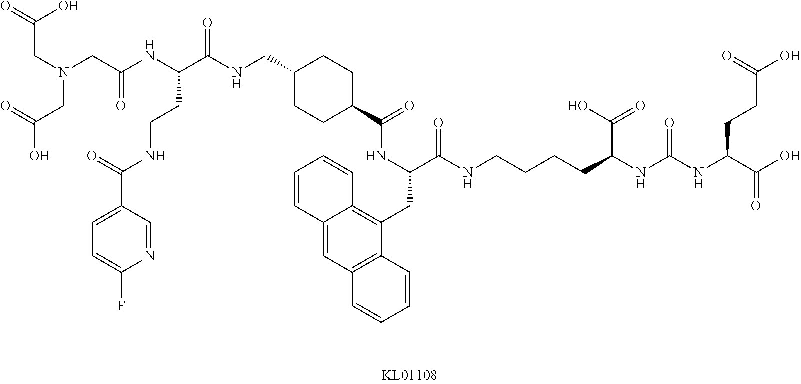 [SMILES: O=C(O)CC[C@H](NC(=O)N[C@@H](CCCCNC(=O)[C@H](Cc1c2ccccc2cc2ccccc12)NC(=O)[C@H]1CC[C@H](CNC(=O)[C@H](CCNC(=O)c2ccc(F)nc2)NC(=O)CN(CC(=O)O)CC(=O)O)CC1)C(=O)O)C(=O)O]

Starting with Fmoc-Lys(ivDde)-Wang resin, urea bond was formed to glutamate for solid phase coupling. The resin then underwent ivDde deprotection, followed by addition of the following sequence Fmoc-L-9-anthrylalanine (5 equivalents), Fmoc-tranexamic acid, Fmoc-Dab(ivDde). Then the Fmoc from the Dab residue is first deprotected to add N,N-bis[2-(tert-butoxy)-2-oxoethyl]glycine. ivDde is then deprotected and the radiolabeling precursor and non-radioactive standard of KL01108 were then generated via coupling N,N,N-trimethyl-5-((2,3,5,6-tetrafluorophenoxy)-carbonyl-pyridine-2-aminiumchloride and 6-fluoronicotinic acid, respectively, according to previous methods. The non-radioactive standard is purified via QC column at 31% MeCN and 0.1% formic acid in water with retention around 10 min. The radiolabeling precursor was purified via HPLC semi-preparative column 30% MeCN and 0.1% formic acid in water with retention around 7 min. The final yields were 10% and 32% for the non-radioactive KL01108 standard and radiolabeling precursor, respectively. The calculated ESI-MS for the non-radioactive KL01108 standard [M+H]⁺ was 1102.4; found 1102.5. The calculated ESI-MS for the radiolabeling precursor [M+H]⁺ was 1142.5; found 1142.3.

### Synthesis of KL01120 and its Radiolabeling Precursor

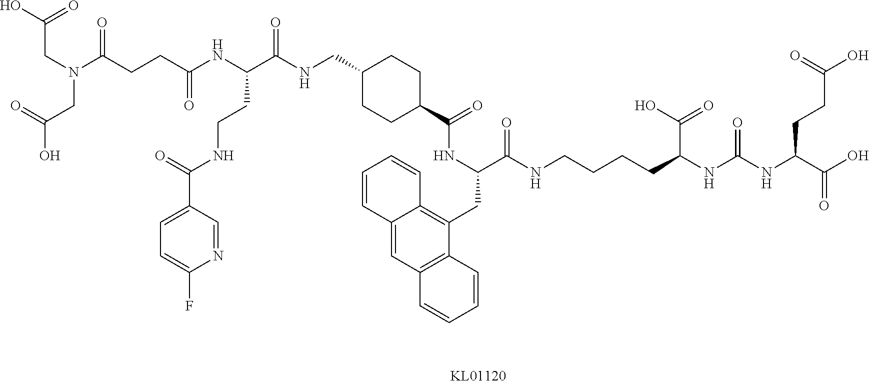 [SMILES: O=C(O)CC[C@H](NC(=O)N[C@@H](CCCCNC(=O)[C@H](Cc1c2ccccc2cc2ccccc12)NC(=O)[C@H]1CC[C@H](CNC(=O)[C@H](CCNC(=O)c2ccc(F)nc2)NC(=O)CCC(=O)N(CC(=O)O)CC(=O)O)CC1)C(=O)O)C(=O)O]

Starting with Fmoc-Lys(ivDde)-Wang resin, urea bond was formed to glutamate for solid phase coupling. The resin then underwent ivDde deprotection, followed by addition of the following sequence Fmoc-L-9-anthrylalanine (5 equivalents), Fmoc-tranexamic acid, Fmoc-Dab(ivDde). Then the Fmoc from the Dab residue is first deprotected to add 4-[bis[2-(1,1-dimethylethoxy)-2-oxoethyl]amino]-4-oxobutanoic acid. ivDde is then deprotected and the radiolabeling precursor and non-radioactive standard of KL01120 were then generated via coupling of N,N,N-trimethyl-5-((2,3,5,6-tetrafluorophenoxy)-carbonyl-pyridine-2-aminiumchloride and 6-fluoronicotinic acid, respectively, according to previous methods. The non-radioactive KL01120 standard was purified with HPLC semi-preparative column: 30% MeCN and 0.1% formic acid in water with retention around 7 min. The radiolabeling precursor was purified on QC column with 25% MeCN and 0.1% formic acid in water with retention around 11.6 min. The final yields were 36% and 22% for the non-radioactive KL01040 standard and radiolabeling precursor, respectively. The calculated ESI-MS for the non-radioactive KL01120 standard [M+H]⁺ was 1144.5; found 1144.5. The calculated ESI-MS for the radiolabeling precursor [M+H]⁺ was 1184.5; found 1184.6.

### Synthesis of KL01130 and its Radiolabeling Precursor

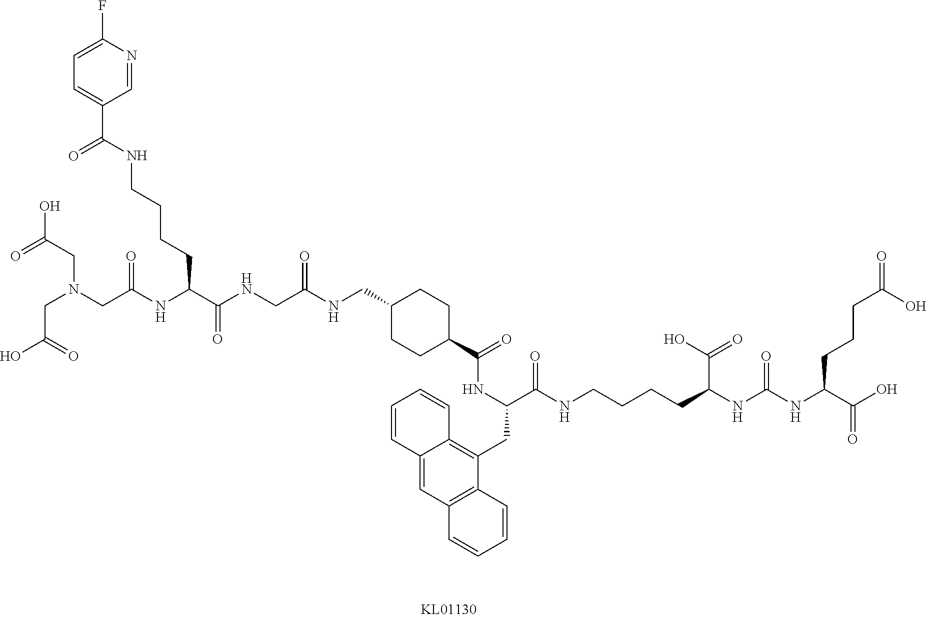 [SMILES: O=C(O)CCC[C@H](NC(=O)N[C@@H](CCCCNC(=O)[C@H](Cc1c2ccccc2cc2ccccc12)NC(=O)[C@H]1CC[C@H](CNC(=O)CNC(=O)[C@H](CCCCNC(=O)c2ccc(F)nc2)NC(=O)CN(CC(=O)O)CC(=O)O)CC1)C(=O)O)C(=O)O]

The PSMA-targeting pharmacophore Lys-urea-Aad (Aad: 2-aminoadipic acid) was synthesized via solid-phase peptide chemistry starting with 1 equivalent of Fmoc-Lys(ivDde)-Wang resin. After swelling in N,N-dimethylformamide (DMF), Fmoc was removed from resin via the treatment of 20% piperidine in DMF. The isocyanate of 2-aminoadipyl component was generated by combining 3-4 equivalents L-2-aminoadipic acid di-tertbutyl ester in dichloromethane and diisopropylethylamine (DIPEA) in dry ice/acetone bath. Triphosgene (3 equivalents) dissolved in dichloromethane (DCM) was added dropwise to the reaction and then warmed to room temperature and stirred for 30 min to generate the isocyanate of 2-aminoadipyl component for addition to resin. The activated Aad was then added to the resin and reacted for 24 hr to form urea linkage. After washing the resin, the ivDde-protection group was removed with 5% hydrazine in DMF, followed by addition of the following sequence Fmoc-L-9-anthrylalanine (5 equivalents), Fmoc-tranexamic acid, and Fmoc-Lys(ivDde). Then the Fmoc from the Lys residue is first deprotected to add Fmoc-Gly and then 4-[bis[2-(1,1-dimethylethoxy)-2-oxoethyl]amino]-4-oxobutanoic acid. ivDde is then deprotected and the radiolabeling precursor and non-radioactive standard of KL01130 were then generated via coupling of N,N,N-trimethyl-5-((2,3,5,6-tetrafluorophenoxy)-carbonyl-pyridine-2-aminiumchloride and 6-fluoronicotinic acid, respectively, according to previous methods. The non-radioactive KL01130 standard was purified with HPLC semi-preparative column: 40% MeCN and 0.1% formic acid in water with retention around 8.8 min. The radiolabeling precursor was purified on HPLC semi-preparative column with 27% MeCN and 0.1% formic acid in water with retention around 9.6 min. The final yields were 23% and 38% for the non-radioactive KL01130 standard and radiolabeling precursor, respectively. The calculated ESI-MS for the non-radioactive KL01120 standard [M+2H]²⁺ was 601.3; found 601.6. The calculated ESI-MS for the radiolabeling precursor [M+2H]²⁺ was 621.3; found 621.3.

### Synthesis of ¹⁸F-KL01040

For the preparation of ¹⁸F-KL01040, H¹⁸F in H₂¹⁸O was passed through QMA column and then eluted with 0.2 mL tetrabutylammonium bicarbonate (0.25 M in 0.2 mL MeCN and water) into a 5 mL V-shaped reaction vessel. Solvent was evaporated at 120° C. and 1 mL of anhydrous MeCN was added and then evaporated again. It was repeated once and then the vessel was capped and cooled down to room temperature. The radiolabeling precursor of KL01040 (2 mg) in 0.4 mL anhydrous DMF was added, and the mixture was incubated at 70° C. for 15 min. The reaction mixture was quenched with 1 mL water and then purified with HPLC (C18 semi-prep column, 4.5 mL/min, 35% MeCN and 0.1% formic acid in water, retention time 14.1 min). Decay corrected radiochemical yield: 25%.

### Synthesis of ¹⁸F-KL01120

For the preparation of ¹⁸F-KL01120, H¹⁸F in H₂¹⁸O was passed through QMA column and then eluted with 0.2 mL tetraethylammonium bicarbonate (0.25 M in 0.2 mL water) into a 5 mL V-shaped reaction vessel. Solvent was evaporated at 120° C. and 1 mL of anhydrous MeCN was added and then evaporated again. It was repeated once and then the vessel was capped and cooled down to room temperature. The radiolabeling precursor of KL01120 (1 mg) in 0.4 mL anhydrous DMF was added and the mixture was incubated at 80° C. for 10 min. The reaction mixture was quenched with 1 mL water and then purified with HPLC (C18 semi-prep column, 4.5 mL/min, 45% MeCN and 0.1% formic acid in water, retention time 10.5 min).

### Synthesis of ¹⁸F-KL01130

For the preparation of ¹⁸F-KL01130, H¹⁸F in H₂¹⁸O was passed through QMA column and then eluted with 0.2 mL tetraethylammonium bicarbonate (0.25 M in 0.2 mL water) into a 5 mL V-shaped reaction vessel. Solvent was evaporated at 120° C. and 1 mL of anhydrous MeCN was added and then evaporated again. It was repeated once and then the vessel was capped and cooled down to room temperature. The radiolabeling precursor of KL01130 (2 mg) in 0.4 mL anhydrous DMF was added and the mixture was incubated at 80° C. for 15 min. The reaction mixture was quenched with 1 mL water and then purified with HPLC (C18 semi-prep column, 4.5 mL/min, 40% MeCN and 0.1% formic acid in water, retention time 10.5 min).

Synthesis of ¹⁸F-KL01007-Pyridine-BF₃

For the preparation of ¹⁸F-KL01007-pyridine-BF₃, H¹⁸F trapped on QMA Sep-Pak was eluted with 0.1 mL 1 M pyridazine HCl solution (pH 2.0) into a 5 mL V-shaped polypropylene reaction vessel sealed with rubber septum, preloaded with 100 nmol of KL01007-pyridine-BF₃ in 15 mL DMF. The reaction mixture was incubated at 80° C. for 5 min and another 15 min while vacuum was applied. The residue was diluted with 1 mL deionized water and injected into HPLC for purification (Luna C18 semi-prep column, flow rate 4.5 mL/min, mobile phase 38% MeCN and 0.1% formic acid in water, retention time 7.5 min). Decay corrected radiochemical yield: 2%.

### Example 2: Biodistribution and PET/CT Imaging Studies in Tumor-Bearing Mice

All imaging and biodistribution studies used male NOD-scid IL2Rg^{null} (NSG) mice and conducted according to the guidelines established by the Canadian Council on Animal Care and approved by Animal Ethics Committee of the University of British Columbia. For tumor inoculations, mice were anesthetized via inhalation of 2% isoflurane in oxygen and implanted subcutaneously with 5×10⁶ LNCaP cells below the left shoulder. Imaging and biodistribution studies were performed only after tumors grew to 5-8 mm in diameter.

For PET/CT imaging studies, approximately 5 MBq of the ¹⁸F-labeled tracer was injected through the tail vein. While for ex vivo biodistribution studies, mice were injected with around 2 MBq of the ¹⁸F-labeled tracer through the tail vein. Mice were allowed to recover and roam freely in the cages after injecting the tracer. At 45 min post-injection, imaging mouse were sedated again and positioned on the scanner. A 10 min CT scan was conducted first to allow for localization and attenuation correction for later reconstruction of PET images. A 10 min PET image was acquired following CT scan. Heating pads were used during the entire procedure to keep the mice warm and circulate the blood. At 1 h post-injection, all mice for biodistribution study were euthanized with blood drawn from heart, and all tissues of interest were collected, rinsed with PBS, blotted dry, weighed, and counted using an automated gamma counter. The uptake in each organ/tissue was normalized to the injected dose and expressed as the percentage of the injected dose per gram of tissue (% ID/g).

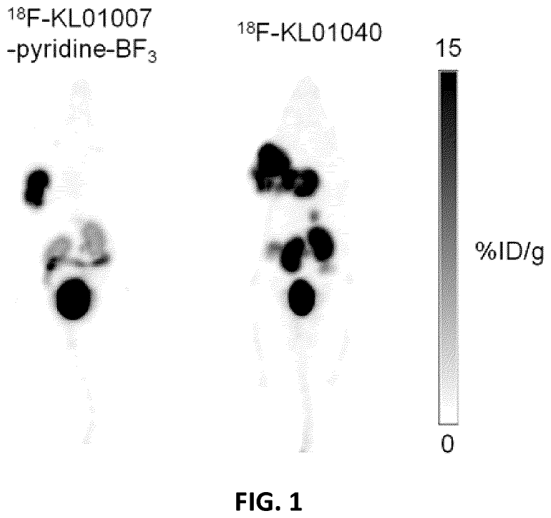

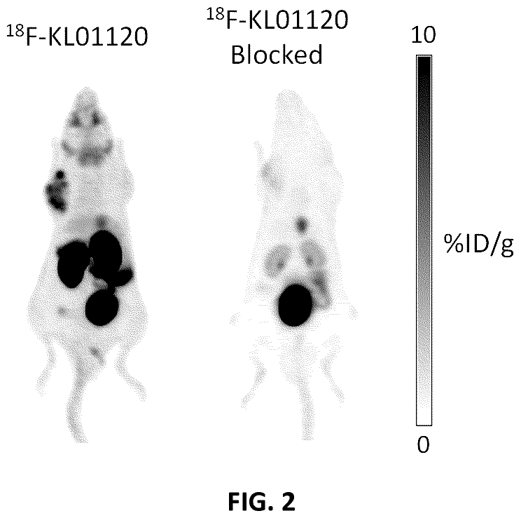

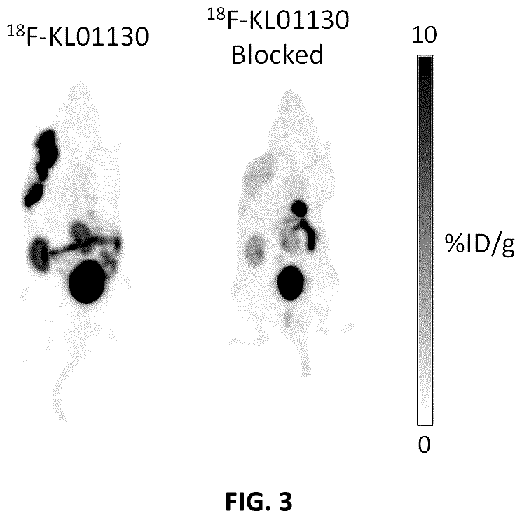

Representative maximum intensity projection PET images of ¹⁸F-KLF1007-pyridine-BF₃ and ¹⁸F-KL01040 acquired at 1 h post-injection from LNCaP tumor-bearing mice is enumerated in Table 4 and shown in FIG. 1. Representative maximum intensity projection PET images of ¹⁸F-KL01120 and ¹⁸F-KL01130 acquired at 1 h post-injection from LNCaP tumor-bearing mice is also enumerated in Table 5 and shown in FIG. 2 (¹⁸F-KL1120) and enumerated in Table 6 and shown in FIG. 3 (¹⁸F-KL01130).

TABLE 4
Biodistribution (at 1 h post-injection) of ¹⁸F-KL01007-pyridine-BF₃
and ¹⁸F-KL01040 in mice bearing LNCaP tumor xenografts.
Data are presented as mean ± SD % ID/g.

| | ¹⁸F-KL01007-pyridine-BF₃ | ¹⁸F-KL01040 |
|---|---|---|
| Blood | 0.79 ± 0.26 | 1.96 ± 0.37 |
| Fat | 0.14 ± 0.05 | 0.61 ± 0.15 |
| Testes | 0.33 ± 0.16 | 0.47 ± 0.07 |
| Seminal Glands | 0.11 ± 0.06 | 0.26 ± 0.06 |
| Small Intestine | 1.69 ± 1.09 | 2.54 ± 0.50 |
| Spleen | 0.35 ± 0.20 | 0.83 ± 0.41 |
| Liver | 0.24 ± 0.08 | 0.58 ± 0.15 |
| Pancreas | 0.14 ± 0.05 | 0.38 ± 0.05 |
| Stomach | 0.14 ± 0.08 | 0.20 ± 0.07 |
| Adrenal Glands | 0.22 ± 0.11 | 1.05 ± 0.26 |
| Kidneys | 7.78 ± 2.15 | 33.2 ± 10.1 |
| Lungs | 0.68 ± 0.18 | 1.56 ± 0.40 |
| Heart | 0.26 ± 0.09 | 0.56 ± 0.14 |
| LNCaP Tumor | 11.2 ± 3.44 | 11.2 ± 2.06 |
| Muscle | 0.13 ± 0.06 | 0.29 ± 0.06 |
| Bone | 0.40 ± 0.08 | 0.60 ± 0.18 |
| Brain | 0.02 ± 0.01 | 0.11 ± 0.08 |
| Tail | 0.07 ± 0.20 | 1.31 ± 0.41 |
| Salivary Glands | 0.23 ± 0.07 | 0.79 ± 0.11 |
| Thyroid | 0.24 ± 0.07 | 0.60 ± 0.06 |
| Lacrimal Glands | 0.05 ± 0.02 | 0.23 ± 0.06 |

TABLE 5
Biodistribution and tumor-to-background uptake ratios of
¹⁸F-KL01120 in LNCaP tumor-bearing mice.
Data are presented as mean ± SD % ID/g.

| Tissue (% ID/g) | 1 h (n = 8) | 1 h Blocked (n = 8) |
|---|---|---|
| Blood | 0.71 ± 0.14 | 0.86 ± 0.20 |
| Fat | 1.57 ± 0.55 | 0.18 ± 0.05 |
| Testes | 0.92 ± 0.29 | 0.28 ± 0.07 |
| Seminal Gland | 0.98 ± 0.42 | 0.76 ± 0.19 |
| Small Intestine | 4.51 ± 0.87 | 1.83 ± 0.37 |
| Large Intestine | 1.11 ± 0.42 | 0.25 ± 0.10 |
| Spleen | 22.1 ± 2.92 | 0.29 ± 0.03 |
| Liver | 2.64 ± 0.57 | 0.59 ± 0.10 |
| Pancreas | 2.22 ± 0.66 | 0.27 ± 0.07 |
| Stomach | 0.68 ± 0.17 | 0.14 ± 0.06 |
| Adrenal Glands | 11.2 ± 4.71 | 0.48 ± 0.21 |
| Kidneys | 88.1 ± 17.4 | 4.29 ± 1.34 |
| Lungs | 4.10 ± 0.71 | 0.81 ± 0.18 |
| Heart | 2.90 ± 0.66 | 0.35 ± 0.06 |
| LNCaP Tumor | 6.21 ± 1.25 | 1.87 ± 0.46 |
| Muscle | 0.90 ± 0.20 | 0.27 ± 0.18 |
| Bone | 0.92 ± 0.46 | 0.35 ± 0.23 |
| Brain | 0.05 ± 0.01 | 0.03 ± 0.00 |
| Salivary Glands | 9.18 ± 2.36 | 0.35 ± 0.08 |
| Thyroid | 4.53 ± 0.74 | 0.33 ± 0.08 |
| Lacrimal Glands | 0.06 ± 0.03 | 0.07 ± 0.05 |
| Tumour:Bone | 9.31 ± 3.39 | 8.46 ± 4.80 |
| Tumor: Muscle | 6.58 ± 1.38 | 9.96 ± 3.13 |
| Tumour:Blood | 8.41 ± 2.02 | 2.21 ± 0.46 |
| Tumour:Kidney | 0.07 ± 0.02 | 0.45 ± 0.05 |

TABLE 6
Biodistribution and tumor-to-background uptake ratios of
¹⁸F-KL01130 in LNCaP tumor-bearing mice.
Data are presented as mean ± SD % ID/g.

| Tissue (% ID/g) | 1 h (n = 5) | 1 h Blocked (n = 5) |
|---|---|---|
| Blood | 1.20 ± 0.26 | 1.78 ± 0.12 |
| Fat | 0.22 ± 0.09 | 0.33 ± 0.10 |
| Testes | 0.29 ± 0.05 | 0.41 ± 0.05 |
| Seminal Gland | 0.15 ± 0.04 | 0.22 ± 0.06 |
| Small Intestine | 2.01 ± 0.60 | 2.40 ± 0.36 |
| Large Intestine | 0.20 ± 0.10 | 0.26 ± 0.10 |
| Spleen | 0.36 ± 0.09 | 0.31 ± 0.06 |
| Liver | 0.47 ± 0.06 | 0.67 ± 0.07 |
| Pancreas | 0.21 ± 0.05 | 0.32 ± 0.04 |
| Stomach | 0.19 ± 0.11 | 0.36 ± 0.15 |
| Adrenal Glands | 0.59 ± 0.06 | 0.39 ± 0.17 |
| Kidneys | 19.0 ± 5.04 | 4.77 ± 0.30 |
| Lungs | 0.96 ± 0.19 | 1.22 ± 0.17 |
| Heart | 0.34 ± 0.07 | 0.45 ± 0.05 |
| LNCaP Tumor | 8.01 ± 0.90 | 2.11 ± 0.02 |
| Muscle | 0.34 ± 0.24 | 0.20 ± 0.02 |
| Bone | 0.52 ± 0.17 | 0.57 ± 0.16 |
| Brain | 0.04 ± 0.01 | 0.04 ± 0.00 |
| Salivary Glands | 0.52 ± 0.18 | 0.49 ± 0.14 |
| Thyroid | 0.38 ± 0.16 | 0.48 ± 0.03 |
| Lacrimal Glands | 0.10 ± 0.06 | 0.04 ± 0.02 |
| Tumour: Bone | 14.6 ± 4.27 | 3.89 ± 1.05 |
| Tumor:Muscle | 35.7 ± 5.75 | 10.4 ± 1.31 |
| Tumour:Blood | 6.93 ± 1.54 | 1.19 ± 0.12 |
| Tumour: Kidney | 0.59 ± 0.35 | 0.44 ± 0.05 |

The present invention has been described with regard to one or more embodiments. However, it will be apparent to persons skilled in the art that a number of variations and modifications can be made without departing from the scope of the invention as defined in the following claims. The scope of the invention should therefore not be limited by the preferred embodiments set forth in the above Examples, but should be given the broadest interpretation consistent with the description as a whole.

1. A compound of Formula I: 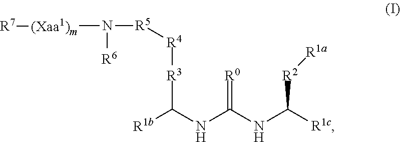
or a salt or a solvate thereof, wherein:
R⁰ is O or S;
R^{1a} is —CO₂H, —SO₂H, —SO₃H, —PO₂H, —PO₃H₂, —OPO₃H₂, —OSO₃H, —B(OH)₂, or 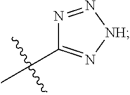 [SMILES: *c1nn[nH]n1]
R^{1b} is —CO₂H, —SO₂H, —SO₃H, —PO₂H, —PO₃H₂, —B(OH)₂, or 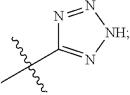 [SMILES: *c1nn[nH]n1]
R^{1c} is —CO₂H, —SO₂H, —SO₃H, —PO₂H, —PO₃H₂, —B(OH)₂, or 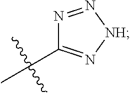 [SMILES: *c1nn[nH]n1]
R² is —CH₂—, —CH(OH)—, —CHF—, —CF₂—, —CH(CH₃)—, —C(CH₃)₂—, —(CH₂)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, or —C(O)—NH—C(CH₃)₂—;
R³ is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic C₂-C₂₀ heteroalkylenyl, heteroalkenylenyl, or heteroalkynylenyl;
R⁴ is —O—, —S—, —Se—, —S(O)—, —S(O)₂—, —NHC(O)—, —C(O)NH—, 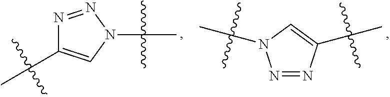 [SMILES: *c1cn(*)nn1 ; *c1cn(*)nn1]
—C(O)—(NH)₂—C(O)—, —OC(O)NH—, —NHC(O)O—, —NHC(O)NH—, —OC(S)NH—, —NHC(S)O—, —NHC(S)NH—, —NHC(O)C(O)NH—, —S—S—, —S—CH₂—S—, —NH—NH—C(O)—, or —C(O)—NH—NH—;
R⁵ is a linear or branched, cyclic or acyclic, and/or aromatic or non-aromatic C₁-C₃₀ alkylenyl, alkenylenyl or alkynylenyl, or is a linear or branched, cyclic or acyclic, and/or aromatic or non-aromatic C₂-C₃₀ heteroalkylenyl, heteroalkenylenyl or heteroalkynylenyl;
R⁶ is hydrogen, methyl or ethyl;
Xaa¹ is an amino acid of formula —N(R⁸)R⁹C(O)—;
each R⁸ is independently hydrogen or methyl;
each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
wherein at least one R⁹ or R⁵ is —(CH₂)₀₋₃CH(R¹⁰)(CH₂)₀₋₃—;
R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic C₂-C₁₉ heteroalkyl, heteroalkenyl or heteroalkynyl having only 1-3 heteroatoms;
—CH₂R²³, in which R²³ is an optionally substituted C₄-C₁₆ aromatic ring or partially or fully aromatic fused ring system, wherein 0-3 carbons in the aromatic ring or the partially or fully aromatic fused ring system are replaced with N, S and/or O heteroatoms, and wherein the optional substitutions are selected from OH, NH₂, NO₂, halogen, C₁-C₆ alkyl, and/or C₁-C₆ alkoxyl groups; or
selected from: 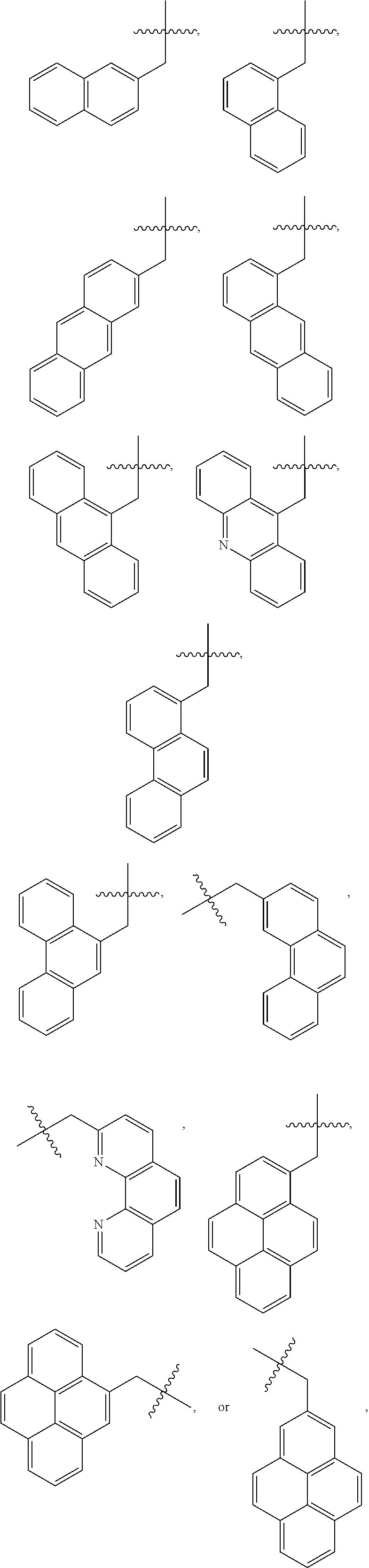
optionally modified with one, more than one, or a combination of: halogen, OMe, SMe, NH₂, NO₂, CN, OH, or additional endocyclic ring nitrogen atoms;
R⁷ is 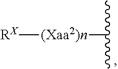
Xaa² is —N(R¹³)R¹⁴C(O)—;
each R¹³ is independently hydrogen or methyl;
each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl; wherein each R¹⁴ is optionally substituted;
wherein each R¹⁴ is optionally substituted with a carboxylic acid, a sulfonic acid, a sulfinic acid, or a phosphoric acid; wherein each R¹⁴ is optionally substituted with one R^{X}; and wherein at least one R¹⁴ is substituted with one R^{X}; and
R^{X} is 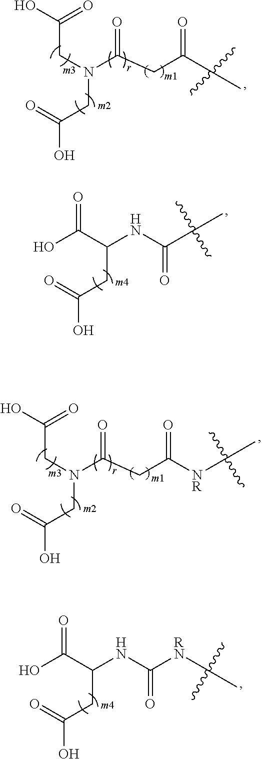
—(C(O))ₚ—(CH₂)_{q}-(radiolabeling group), or —(N(R)C(O))ₚ—(CH₂)_{q}-(radiolabeling group), wherein the radiolabeling group independently selected from: an aryl or heteroaryl substituted with a radioisotope or a prosthetic group comprising a trifluoroborate;
R is hydrogen or methyl;
wherein at least one R^{X} is 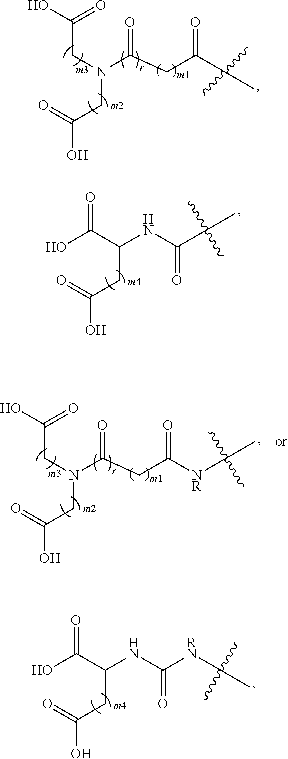
and at least one other R^{X} is —(C(O))ₚ—(CH₂)_{q}-(radiolabeling group) or —(N(R)C(O))ₚ—(CH₂)_{q}-(radiolabeling group);
r is 0 or 1;
n is 1, 2, 3, or 4;
m is 1, 2, 3, or 4;
p is 0 or 1;
q is 0, 1, 2, 3, 4, or 5; and
m1, m2, m3, and m4 are each independently 1, 2, 3, or 4.

2. The compound of claim 1, wherein R⁰ is O.

3. The compound of claim 1 or 2, wherein:
R^{1a} is —CO₂H, —SO₂H, —SO₃H, —PO₂H, —PO₃H₂, —OPO₃H₂, —OSO₃H;
R^{1b} is —CO₂H, —SO₂H, —SO₃H, —PO₂H, —PO₃H₂; and
R^{1c} is —CO₂H, —SO₂H, —SO₃H, —PO₂H, —PO₃H₂.

4. The compound of claim 1 or 2, wherein each of R^{1a}, R^{1b} and R^{1c} is —CO₂H.

5. The compound of any one of claims 1 to 4, wherein R² is —CH₂—, —CH(OH)—, —CHF—, —CF₂—, —CH(CH₃)—, —(CH₂)₂—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

6. The compound of any one of claims 1 to 4, wherein R² is —(CH₂)₂— or —(CH₂)₃—.

7. The compound of any one of claims 1 to 4, wherein R² is —CH₂—, —(CH₂)₂—, —(CH₂)₃—, —CH₂CHF—, —CHFCH₂—, —CH₂OCH₂—, or —CH₂SCH₂—.

8. The compound of any one of claims 1 to 4, wherein R² is —CH₂CHF—, wherein the second carbon in R² has R-configuration.

9. The compound of any one of claims 1 to 8, wherein R³ is a linear or branched, cyclic or acyclic, and/or aromatic or non-aromatic C₁-C₁₀ alkylenyl or alkenylenyl, or a linear or branched, cyclic or acyclic, and/or aromatic or non-aromatic C₂-C₁₀ heteroalkylenyl or heteroalkenylenyl.

10. The compound of any one of claim 1 to 9, wherein R³ is a linear or branched C₁-C₆ alkylenyl or a linear or branched C₂-C₆ heteroalkylenyl.

11. The compound of any one of claims 1 to 10, wherein R³ is: —CH₂—; —(CH₂)₂—; —(CH₂)₃; —(CH₂)₄—; —(CH₂)₅—; —CH₂—O—CH₂—; —CH₂—O—CH₂—CH₂—; —CH₂—CH₂—O—CH₂—; or —CH₂—S—CH₂—.

12. The compound of claim 11, wherein R³ is —(CH₂)₄—; —CH₂—O—CH₂—CH₂—; or —CH₂—CH₂—O—CH₂—.

13. The compound of any one of claims 1 to 12, wherein R⁴ is —O—, —S—, —S(O)—, —S(O)₂—, —NHC(O)—, —C(O)NH—,

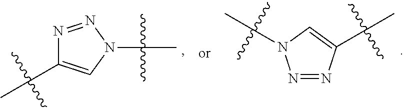 [SMILES: *c1cn(*)nn1 ; *c1cn(*)nn1]

14. The compound of any one of claims 1 to 13, wherein R⁴ is —NHC(O)— or —C(O)NH—.

15. The compound of any one of claims 1 to 14, wherein at least one R⁹ or R⁵ is

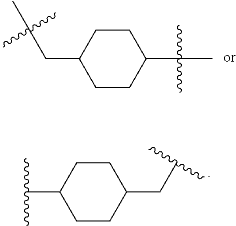 [SMILES: *CC1CCC(*)CC1 ; *CC1CCC(*)CC1]

16. The compound of any one of claims 1 to 15, wherein R⁵ is —(CH₂)₀₋₃CH(R¹⁰)(CH₂)₀₋₃—.

17. The compound of any one of claims 1 to 16, wherein R⁵ is —CH(R¹⁰)—.

18. The compound of any one of claims 1 to 17, wherein R¹⁰ is 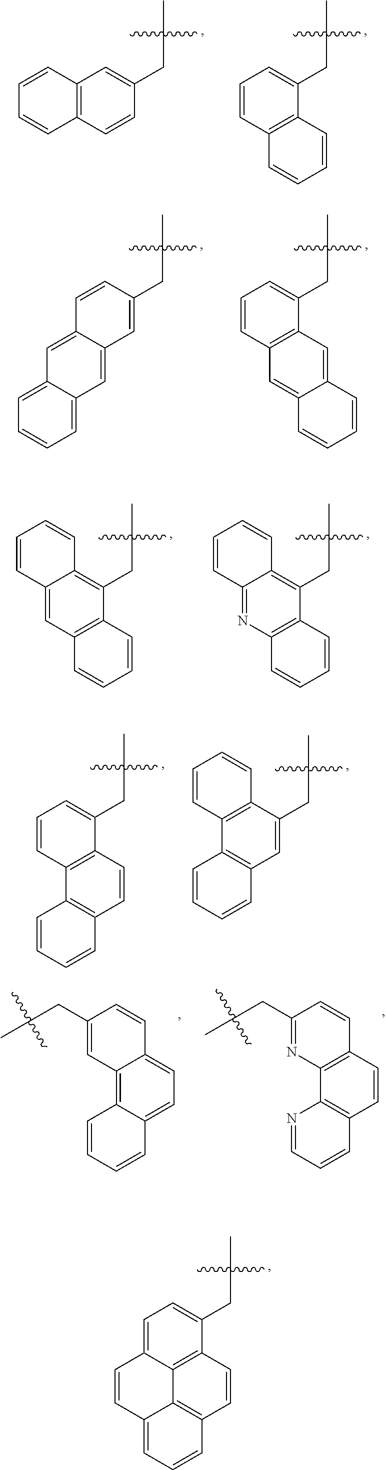 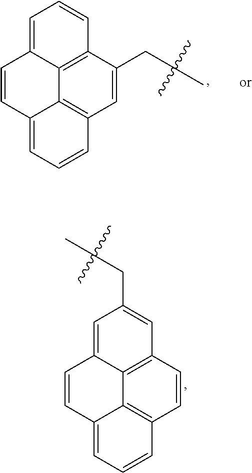 [SMILES: *Cc1cc2cccc3ccc4cccc1c4c32 ; *Cc1cc2ccc3cccc4ccc(c1)c2c34]
each optionally modified with one, more than one, or a combination of: halogen, OMe, SMe, NH₂, NO₂, CN, OH, or additional endocyclic ring nitrogen atoms.

19. The compound of any one of claims 1 to 18, wherein R¹⁰ is

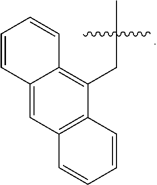 [SMILES: *Cc1c2ccccc2cc2ccccc12]

20. The compound of any one of claims 1 to 19, wherein —(Xaa¹)ₘN(R⁶)R⁵R⁴— is

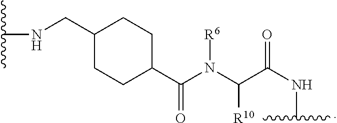

21. The compound of any one of claims 1 to 20, wherein R⁶ is hydrogen.

22. The compound of any one of claims 1-21, wherein R⁷ is

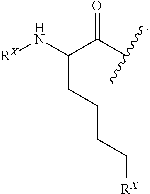

23. The compound of any one of claims 1-21, wherein n is 3 and each Xaa² is independently selected from:

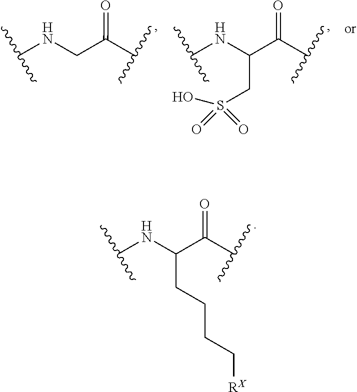

24. The compound of any one of claims 1-21, wherein R⁷ is

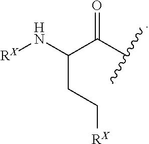

25. The compound of any one of claims 1 to 24, wherein at least one R^{X} is

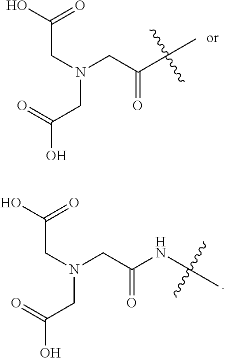 [SMILES: *C(=O)CN(CC(=O)O)CC(=O)O ; *NC(=O)CN(CC(=O)O)CC(=O)O]

26. The compound of an one of claims 1 to 24, wherein at least one R^{X} is

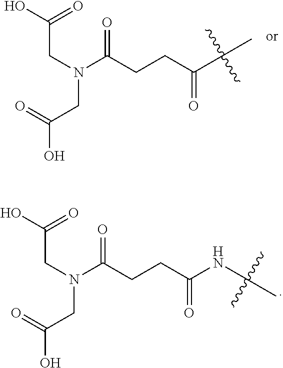 [SMILES: *C(=O)CCC(=O)N(CC(=O)O)CC(=O)O ; *NC(=O)CCC(=O)N(CC(=O)O)CC(=O)O]

27. The compound of any one of claims 1 to 26, wherein at least one R^{X} is a prosthetic group comprising a trifluoroborate.

28. The compound of any one of claims 1 to 26, wherein at least one R^{X} is an aryl group substituted with a radioisotope.

29. The compound of claim 28, wherein:
R^{x} is 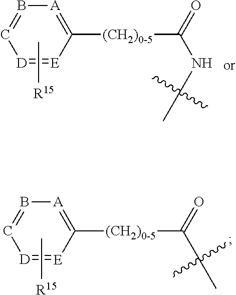
A, B, C, D and E are independently CH or N; and
R¹⁵ is a radioisotope.

30. The compound of claim 29, wherein R^{X} is selected from the group consisting of:

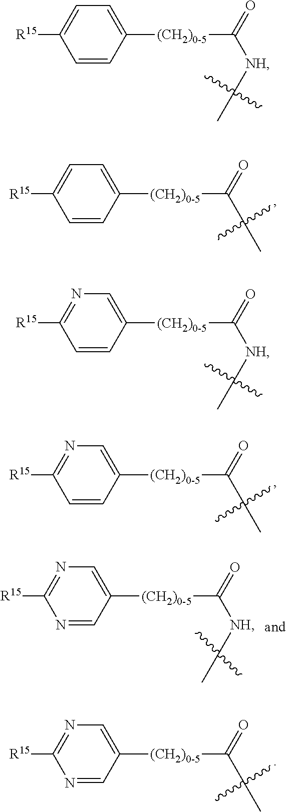

31. The compound of claim 29 or 30, wherein R^{X} is selected from the group consisting of

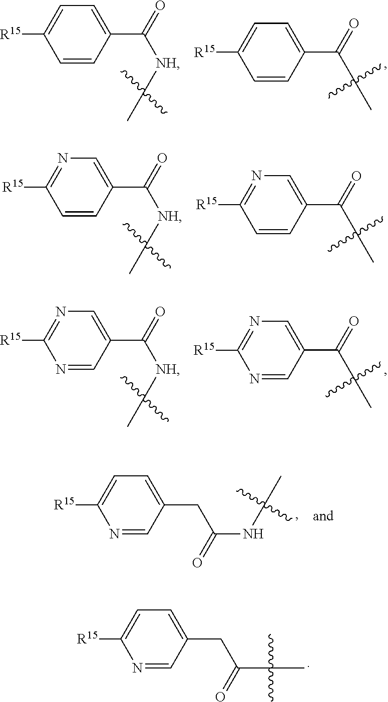

32. The compound of any one of claims 29 to 31, wherein R^{X} is

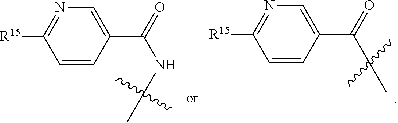

33. The compound of any one of claims 29 to 32, wherein R¹⁵ is independently ¹³¹I, ¹²⁴I, ¹²³I, ⁷⁶Br or ¹⁸F.

34. The compound of any one of claims 29 to 33, wherein R¹⁵ is ¹⁸F.

35. The compound of claim 27, wherein the trifluoroborate is capable of ¹⁸F/¹⁹F exchange radiolabeling.

36. The compound of any one of claims 1 to 27 and 35, wherein:
R^{X} is —(N(R)C(O))ₚ—(CH₂)_{q}—(R¹⁶R¹⁷BF₃);
each R¹⁶ is independently absent, 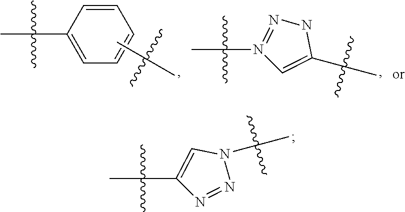
and
—R¹⁷BF₃ is independently be selected from Table 2, Table 3, or 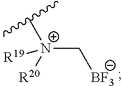
and
R¹⁹ and R²⁰ are each independently linear or branched C₁-C₅ alkyl.

37. The compound of any one of claims 1 to 27 and 35 to 36, wherein:
R^{X} is —(N(R)C(O))ₚ—(CH₂)_{q}—(R¹⁶R¹⁷BF₃);
each R¹⁶ is independently 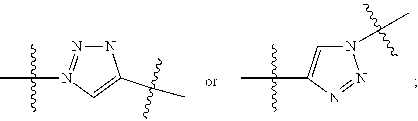
and
R¹⁷BF₃ is

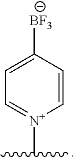

38. The compound of any one of claims 1 to 27 and 35 to 37, wherein at least one fluorine is ¹⁸F.

39. The compound of any one of claims 1 to 38, selected from the group consisting of: 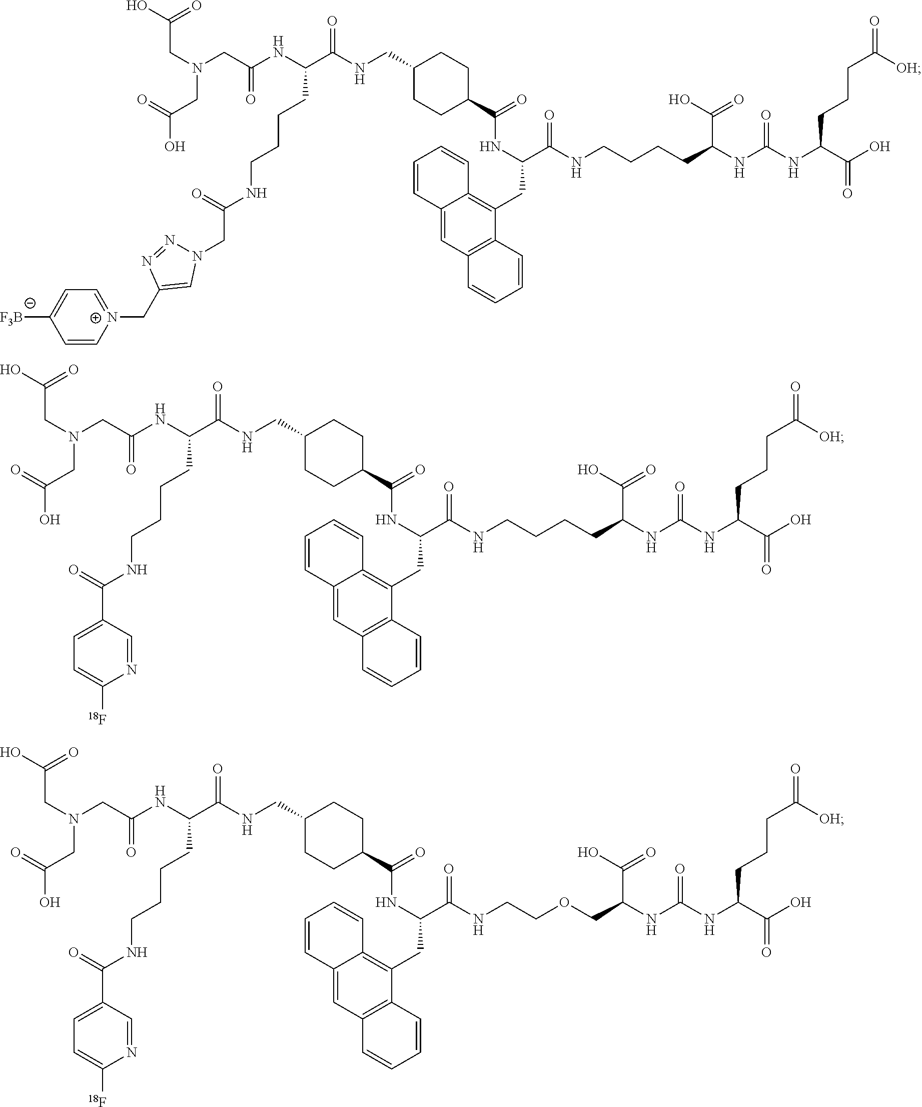 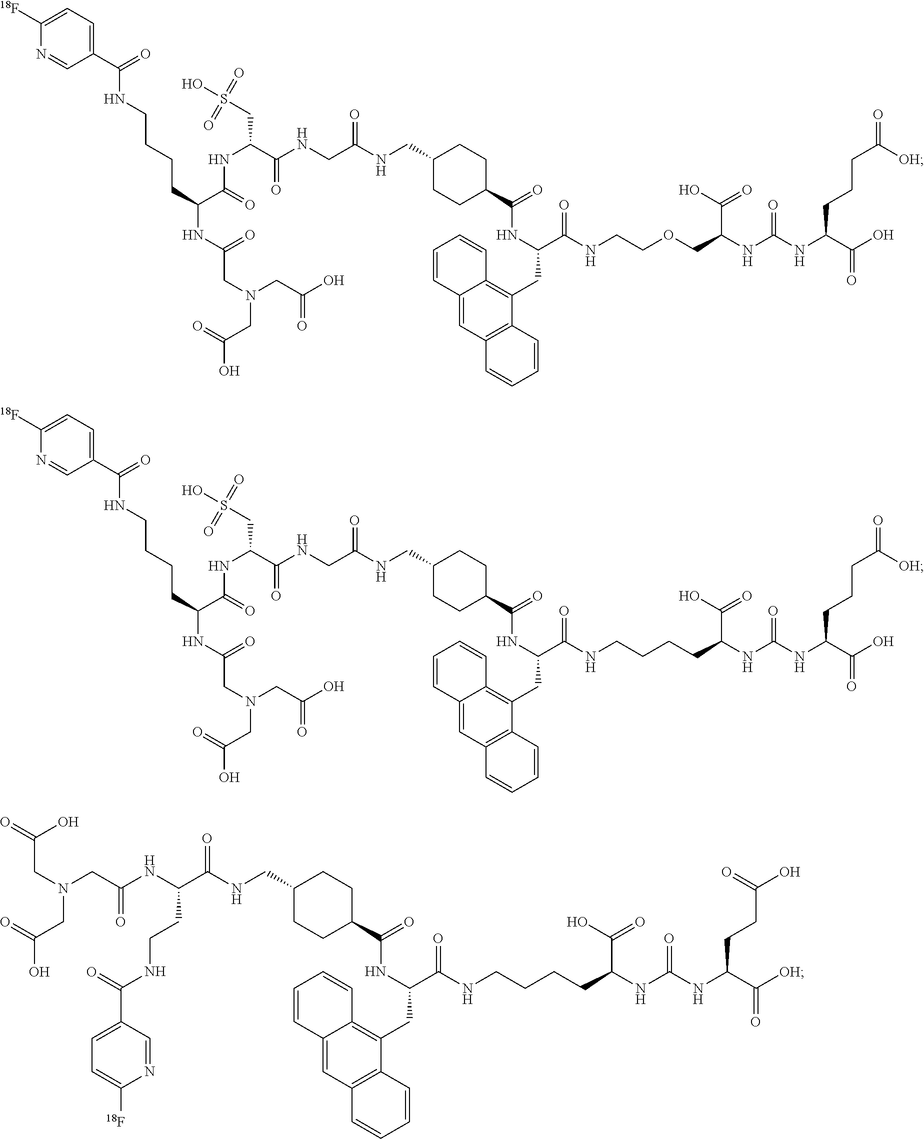 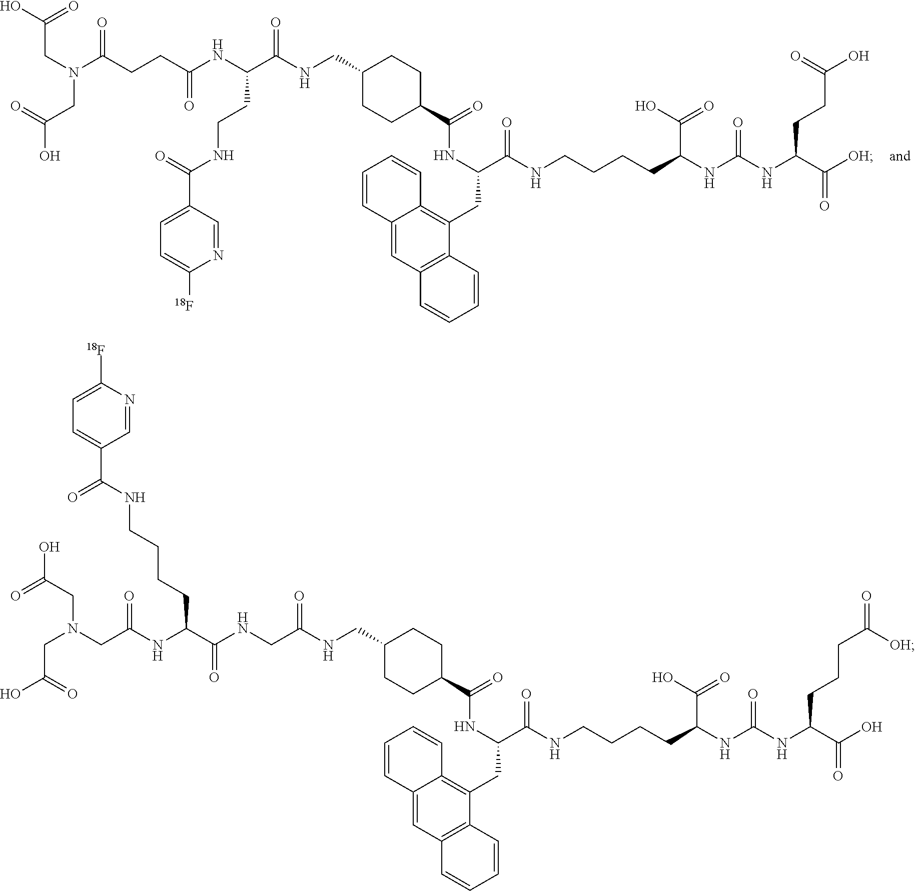 [SMILES: O=C(O)CC[C@H](NC(=O)N[C@@H](CCCCNC(=O)[C@H](Cc1c2ccccc2cc2ccccc12)NC(=O)[C@H]1CC[C@H](CNC(=O)[C@H](CCNC(=O)c2ccc([18F])nc2)NC(=O)CCC(=O)N(CC(=O)O)CC(=O)O)CC1)C(=O)O)C(=O)O ; O=C(O)CCC[C@H](NC(=O)N[C@@H](CCCCNC(=O)[C@H](Cc1c2ccccc2cc2ccccc12)NC(=O)[C@H]1CC[C@H](CNC(=O)CNC(=O)[C@H](CCCCNC(=O)c2ccc([18F])nc2)NC(=O)CN(CC(=O)O)CC(=O)O)CC1)C(=O)O)C(=O)O]
or a salt or solvate thereof.

40. The compound of any one of claims 1 to 38, wherein the compound is 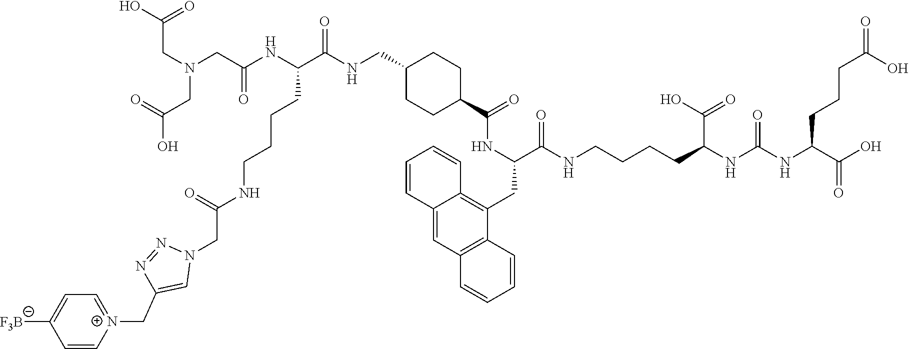
or a salt or solvate thereof;
wherein at least one fluorine is ¹⁸F.

41. The compound of claim 1, wherein the compound has the structure of Formula II: 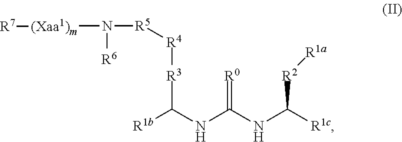
or is a salt or a solvate thereof; wherein:
R⁰ is O or S;
R^{1a} is —CO₂H, —SO₂H, —SO₃H, —PO₂H, —PO₃H₂, —OPO₃H₂, or —OSO₃H;
R^{1b} is —CO₂H, —SO₂H, —SO₃H, —PO₂H, or —PO₃H₂;
R¹⁰ is —CO₂H, —SO₂H, —SO₃H, —PO₂H, or —PO₃H₂, R² is —CH₂—, —(CH₂)₂—, —(CH₂)₃—, —CH₂CHF—, —CHFCH₂—, —CH₂OCH₂—, or —CH₂SCH₂—;
R³ is a linear or branched C₁-C₂₀ alkylenyl or alkenylenyl, or a linear or branched C₂-C₂₀ heteroalkylenyl or heteroalkenylenyl;
R⁴ is —O—, —S—, —S(O)—, —S(O)₂—, —NHC(O)—, —C(O)NH—, 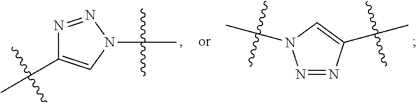 [SMILES: *c1cn(*)nn1 ; *c1cn(*)nn1]
R⁵ is —(CH₂)₀₋₃CH(R¹⁰)(CH₂)₀₋₃—;
R¹⁰ is 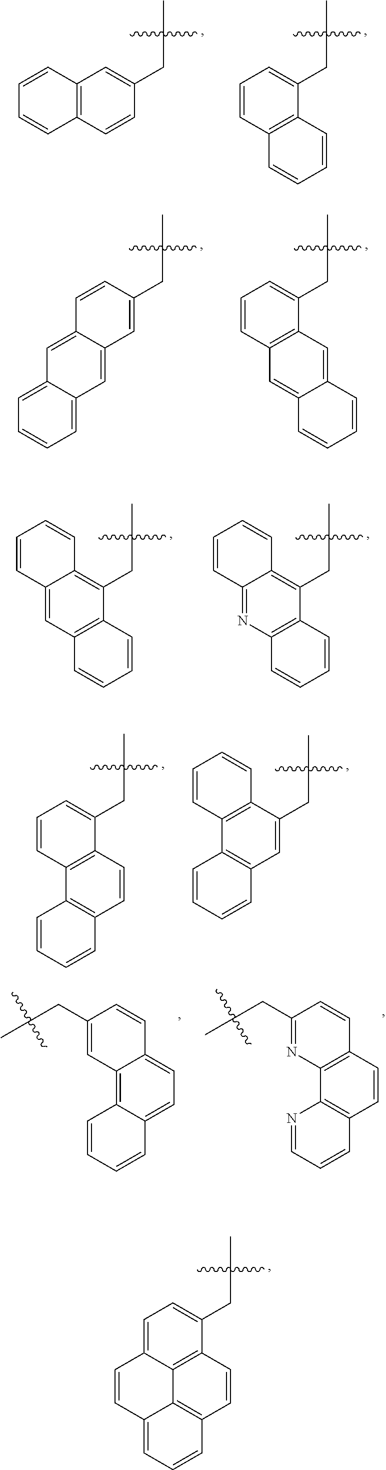 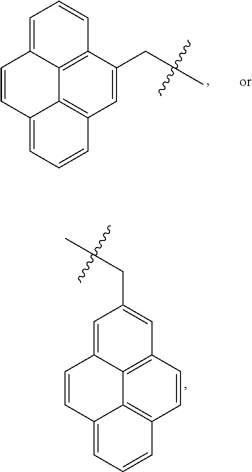 [SMILES: *Cc1cc2cccc3ccc4cccc1c4c32 ; *Cc1cc2ccc3cccc4ccc(c1)c2c34]
optionally modified with one, more than one, or a combination of: halogen, OMe, SMe, NH₂, NO₂, CN, OH, or additional endocyclic ring nitrogen atoms;
R⁶ is hydrogen, methyl or ethyl;
Xaa¹ is an amino acid of formula —N(R⁸)R⁹C(O)—,
each R⁸ is independently hydrogen or methyl;
each R⁹ is independently: -(a linear or branched C₁-C₁₀ alkylenyl or alkenylenyl)-cycloalkylene-, or -(a linear or branched C₂-C₁₀ heteroalkylenyl or heteroalkenylenyl)-cycloalkylene-;
R⁷ is 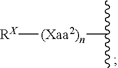
Xaa² is —N(R¹³)R¹⁴C(O)—;
each R¹³ is independently hydrogen or methyl;
each R¹⁴ is independently: a linear or branched C₁-C₂₀ alkylenyl or alkenylenyl, or a linear or branched C₂-C₂₀ heteroalkylenyl or heteroalkenylenyl;
wherein each R¹⁴ is optionally substituted with a carboxylic acid, a sulfonic acid, a sulfinic acid, a phosphoric acid, a hydroxyl, or an amide;
wherein each R¹⁴ is optionally substituted with one R^{X};
wherein at least one R¹⁴ is substituted with one R^{X};
R^{X} is 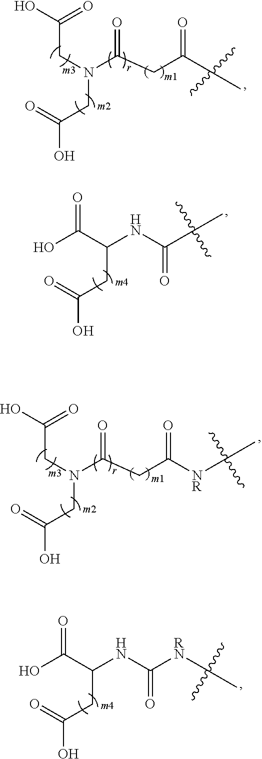
—(C(O))ₚ—(CH₂)_{q}-(radiolabeling group), or —(N(R)C(O))ₚ—(CH₂)_{q}-(radiolabeling group), wherein the radiolabeling group independently selected from: an aryl or heteroaryl substituted with a radioisotope or a prosthetic group comprising a trifluoroborate;
R is hydrogen or methyl;
wherein at least one R^{X} is 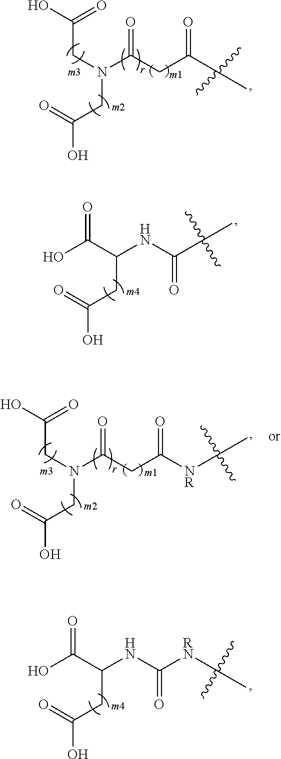
and at least one other R^{X} is —(C(O))ₚ—(CH₂)_{q}-(radiolabeling group) or —(N(R)C(O))ₚ—(CH₂)_{q}-(radiolabeling group);
r is 0 or 1;
n is 1, 2, 3, or 4;
m is 1, 2, 3, or 4;
p is 0 or 1;
q is 0, 1, 2, 3, 4, or 5; and
m1, m2, m3, and m4 are each independently 1, 2, 3, or 4.

42. The compound of claim 1, wherein the compound has the structure of Formula III: 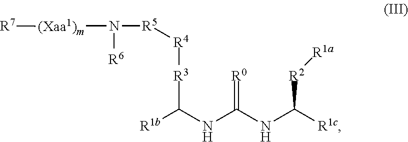
or is a salt or a solvate thereof; wherein:
R⁰ is O;
R^{1a} is —CO₂H;
R^{1b} is —CO₂H;
R¹⁰ is —CO₂H;
R² is —(CH₂)₂— or —(CH₂)₃—;
R³ is —CH₂—, —(CH₂)₂—, —(CH₂)₃, —(CH₂)₄—, —(CH₂)₅—, —CH₂—O—CH₂—, —CH₂—O—CH₂—CH₂—, —CH₂—CH₂—O—CH₂—, or —CH₂—S—CH₂—;
R⁴ is —NHC(O)— or —C(O)NH—;
R⁵ is —(CH₂)₀₋₃CH(R¹⁰)(CH₂)₀₋₃—, wherein R¹⁰ is selected from: 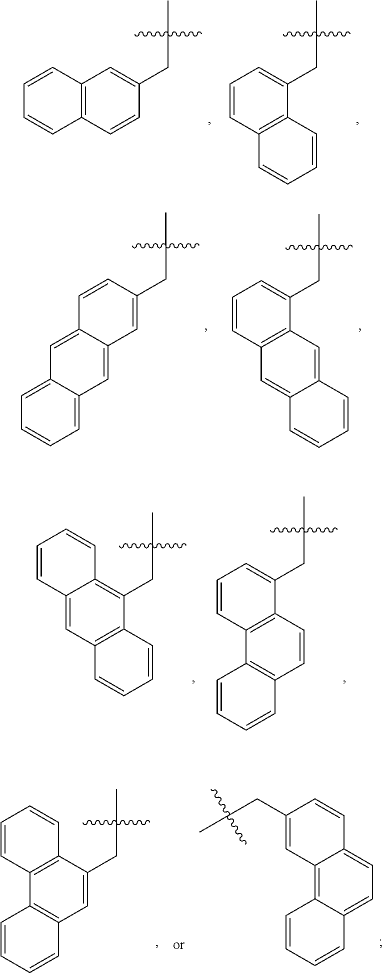
R⁶ is hydrogen or methyl;
Xaa¹ is an amino acid of formula —N(R⁸)R⁹C(O)—;
each R⁸ is independently hydrogen or methyl; and
each R⁹ is independently 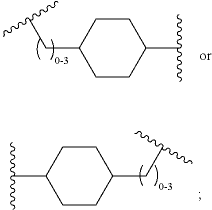
R⁷ is 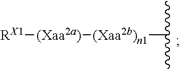
Xaa^{2a} is 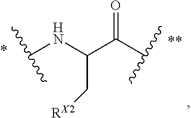
wherein * end connects to R^{X1} and ** end connects to Xaa^{2b};
Xaa^{2b} is *—N(R¹³)R¹⁴C(O)—**, wherein * end connects to Xaa^{2a};
each R¹³ is independently hydrogen or methyl;
each R¹⁴ is independently: a linear or branched C₁-C₆ alkylenyl or alkenylenyl, or a linear or branched C₂-C₆ heteroalkylenyl or heteroalkenylenyl; wherein each R¹⁴ is optionally substituted with a carboxylic acid, a sulfonic acid, a sulfinic acid, a phosphoric acid, a hydroxyl, or an amide;
R^{X1} is 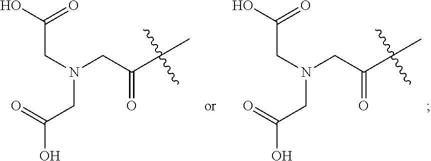 [SMILES: *C(=O)CN(CC(=O)O)CC(=O)O ; *C(=O)CN(CC(=O)O)CC(=O)O]
R^{X2} is —(N(R)C(O))ₚ—(CH₂)_{q}-(radiolabeling group), wherein the radiolabeling group independently selected from: an aryl or heteroaryl substituted with a radioisotope or a prosthetic group comprising a trifluoroborate;
R is hydrogen or methyl;
n1 is 0, 1, 2, or 3;
m is 1, 2, 3, or 4;
p is 0 or 1; and
q is 0, 1, 2, 3, 4, or 5.

43. The compound of any one of claims 1 to 42, wherein the radioisotope is ¹⁸F, ⁷⁶Br, ¹²³I, ¹²⁴I, or ¹³¹I.

44. A compound selected from: 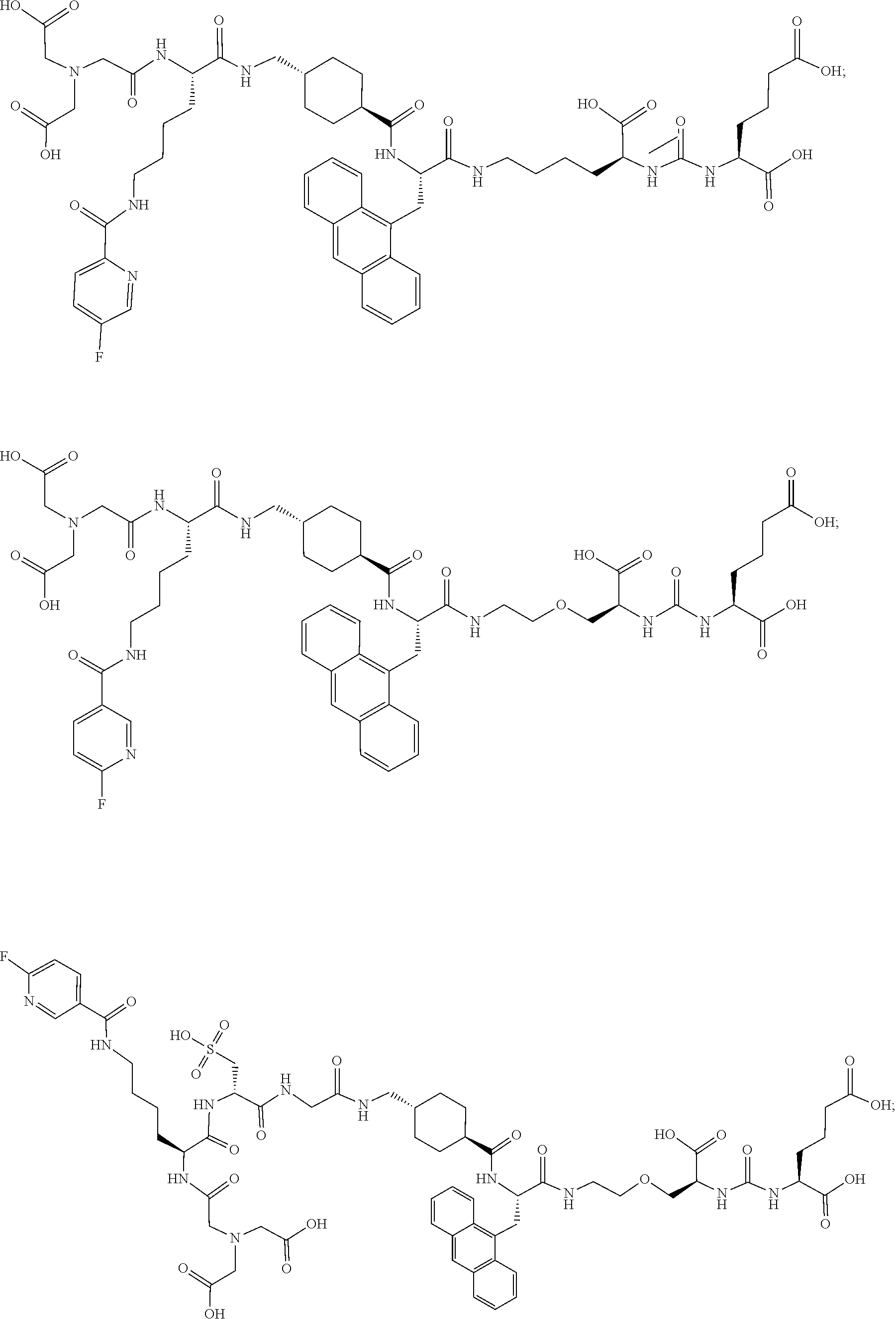 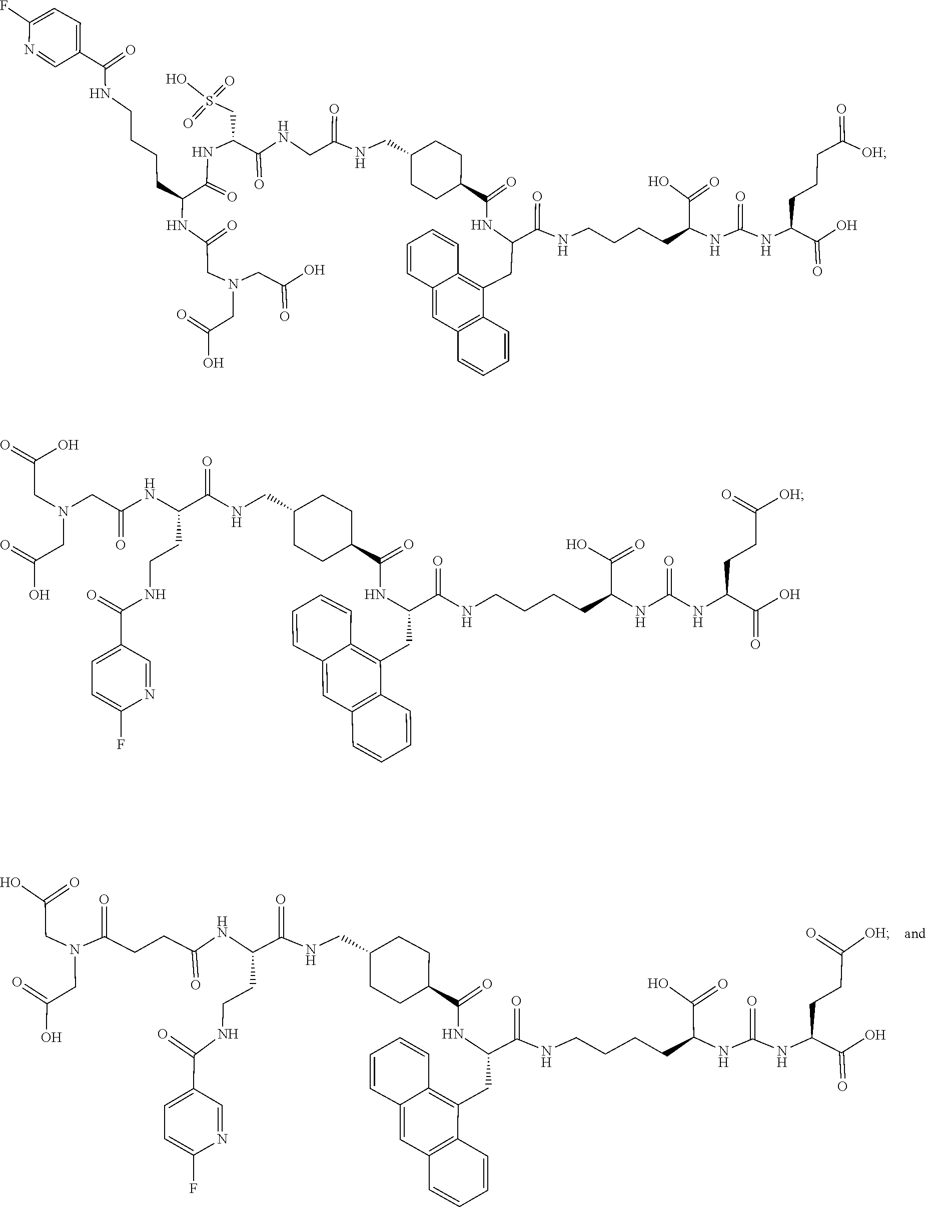 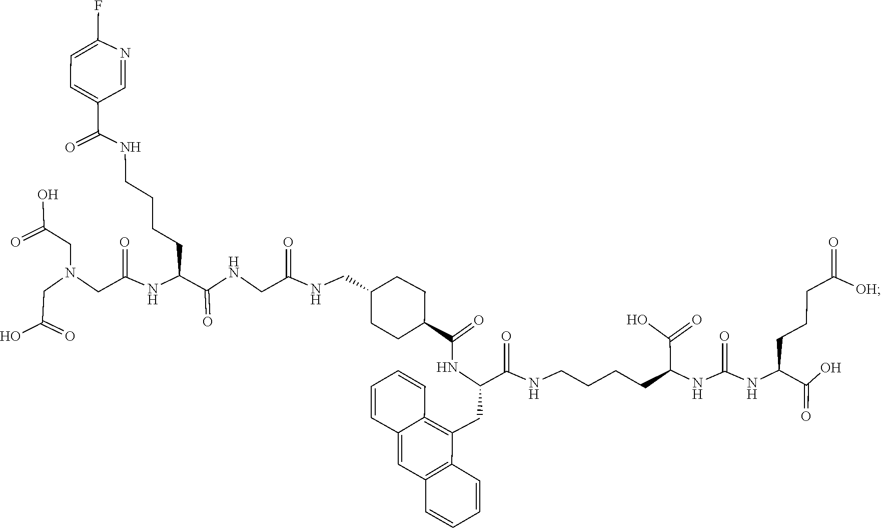 [SMILES: O=C(O)CCC[C@H](NC(=O)N[C@@H](CCCCNC(=O)[C@H](Cc1c2ccccc2cc2ccccc12)NC(=O)[C@H]1CC[C@H](CNC(=O)CNC(=O)[C@H](CCCCNC(=O)c2ccc(F)nc2)NC(=O)CN(CC(=O)O)CC(=O)O)CC1)C(=O)O)C(=O)O]
or is a salt or a solvate thereof.

45. The compound of any one of claims 1 to 44 for use in imaging prostate specific membrane antigen (PSMA)-expressing tissues in a subject, wherein the compound comprises a positron or gamma emitting radioisotope.

46. The compound of claim 45, wherein the positron or gamma emitting radioisotope is ¹⁸F, ⁷⁶Br, ¹²³I, ¹²⁴I, or ¹³¹I.

47. The compound of claim 45, wherein the positron or gamma emitting radioisotope is ¹⁸F.

48. A method of imaging prostate specific membrane antigen (PSMA)-expressing tissues in a subject, comprising:
administering the compound of any one of claims 1 to 44 to a subject in need thereof, wherein the compound comprises a positron or gamma emitting radioisotope; and
imaging the PSMA-expressing tissue.

49. The method of claim 48, wherein the positron or gamma emitting radioisotope is ¹⁸F, ⁷⁶Br, ¹²³I, ¹²⁴I, or ¹³¹I.

50. The method of claim 48, wherein the positron or gamma emitting radioisotope is ¹⁸F.
